(19) United States
(12) Patent Application Publication
(10) Pub. No.: US 2008/0199443 A1
(43) Pub. Date: Aug. 21, 2008

(54) Bone Morphogenetic Variants, Compositions and Methods of Treatment

(75) Inventors: Malcolm Moos, Bethesda, MD (US); J. Terrig Thomas, Arlington, VA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Govt. of the U.S.A. as represented by the Secretary, Dept. of Health and Human Services, Rockville, MD (US)

(21) Appl. No.: 11/916,990

(22) PCT Filed: Jun. 8, 2006
(86) PCT No.: PCT/US06/22806

§ 371 Date: Dec. 7, 2007

Related U.S. Application Data
(60) Provisional application No. 60/689,346, filed on Jun. 9, 2005.

(51) Int. Cl. A61K 35/12; C12N 15/11; C12N 15/00; C12N 5/06; C12N 1/20; A61P 19/00; C12P 21/04; C07K 14/00; A61K 31/70; A61K 38/00

(52) U.S. Cl. 424/93.21; 536/23.5; 435/320.1; 435/325; 435/252.3; 536/24.5; 435/69.1; 530/350; 514/44; 514/12

(57) ABSTRACT

Human cartilage-derived morphogenetic protein variant polypeptides and isolated nucleic acids are provided. Also provided are vectors, host cells, and recombinant methods for producing human cartilage-derived morphogenetic protein variants polypeptides. Therapeutic methods useful for treating musculoskeletal disorders and joint repair with such variants are also provided.

### FIELD

The present invention generally relates to the field of skeletal development. More specifically, the present invention relates to cartilage-derived morphogenetic protein variants that stimulate development and repair of cartilage in vitro and in vivo. The present invention is also directed to the treatment of musculoskeletal disorders and joint repair with such variants.

### BACKGROUND

Morphogenetic proteins are able to induce the proliferation and differentiation of progenitor cells into functional bone, cartilage, tendon, and/or ligament tissue. This class of proteins includes members of the family of bone morphogenetic proteins (BMPs) identified by their ability to induce ectopic, endochondral bone morphogenesis.

The biochemical pathways that control embryonic pattern formation, tissue specification, and injury repair processes occurring postnatally share many common characteristics. Accordingly, several molecular entities that function primarily during embryogenesis under normal circumstances are being evaluated for therapeutic potential. Prominent among these are the BMPs (for reviews see Derynck and Zhang, Nature 425: 577-584, 2003; Seidah and Chretien, Brain Res 848: 45-62, 1999). The BMPs comprise a large class of cystine knot-containing proteins that form homo- or heterodimers that are processed by Subtilisin-like Proprotein Convertases (SPCs) to yield mature, secreted signaling molecules.

Several studies have explored the ability of various SPCs to activate members of the TGF-β superfamily by cleavage at a characteristic RXXR site that divides the mature peptide from the amino-terminal “pro” region (sometimes called the “canonical” proteolytic processing site). SPC1, also known as Furin, enhances the processing of TGF-β1 (Dubois, J Biol Chem 270: 10618-10624, 1995) SPC1 and SPC4 are necessary and sufficient to promote Nodal maturation Beck, Nat Cell Biol 4: 981-985, 2002) and Furin, SPC4, SPC6, or SPC7 can process BMP4 (Cui, EMBO J. 17: 4735-4743, 1998).

Moreover, several BMPs are co-expressed with different members of the SPC family. BMP2, 4, and 7 are coexpressed with SPC4 in the primitive heart, in the apical ectodermal ridge of developing limb buds, and in the interdigital mesenchyme of embryonic limbs (Constam, J Cell Biol 134: 181-191, 1996). During neural tube patterning, SPC6 colocalizes with BMP4 and BMP7 in the dorsal surface ectoderm, whereas SPC4 is co-expressed with BMP6 in the floor plate (Constam, 1996). The promiscuity with which many different BMPs can be cleaved by different SPCs in tissues where they are not normally expressed has been beneficial in attributing potential function based on Xenopus injection assays. For example, overexpression of BMP2, 4 and 7 in early mesoderm induces ventral fates (Dale and Jones, Bioessays 21: 751-760, 1999).

The availability of large amounts of purified and highly active morphogenic proteins would revolutionize procedures generally involving joint repair. Many of the mammalian BMP-encoding genes are now cloned and can be expressed recombinantly as active homo- and heterodimeric proteins in a variety of host systems, including bacteria. The ability to produce active forms of morphogenic proteins such as BMPs recombinantly, including variants and mutants with increased bioactivities, including increased processing efficiency, makes potential therapeutic treatments using morphogenic proteins feasible.

Given the potential therapeutic uses for morphogenic proteins in bone and joint repair, and in view of their inefficient processing in vivo, there is a need for morphogenic proteins that are processed more efficiently. It would thus be desirable to increase certain properties of morphogenic proteins.

Cartilage-derived morphogenetic protein-1 (CDMP-1), a member of the BMP family, has been implicated in the proper growth and differentiation of skeletal tissues, correct anatomic patterning of the joints, the structures comprising them, and the limb skeleton generally. Accordingly it is being evaluated as a therapeutic candidate in various joint repair indications. One method for delivering CDMP-1 is via cells expressing it. However, under normal conditions, the protein is processed much more inefficiently than other BMPs. A need remains for genetic modifications of cell populations that express CDMP-1 variants to enhance production of the mature protein, thereby enhancing their effectiveness in bone and joint repair.

### SUMMARY

The present invention therefore provides nucleic acids encoding human cartilage-derived morphogenetic protein-1 (hCDMP-1) variant polypeptides and isolated nucleic acids. The invention therefore provides methods of screening for variants. The invention further provides vectors, host cells, and recombinant methods for producing human cartilage-derived morphogenetic protein variants polypeptides. Therapeutic methods and reagents useful for treating musculoskeletal disorders and joint repair with such variants are also provided.

In one aspect, the a recombinant polynucleotide comprising a human Cartilage Derived Morphogenetic Protein-1 (hCDMP-1) variant or homolog thereof is provided, wherein the recombinant polynucleotide is (a) a polynucleotide that has the sequence of SEQ ID NO: 3; (b) a polynucleotide that hybridizes under stringent hybridization conditions to (a) and encodes an amino acid sequence of SEQ ID NO: 4; or (c) a polynucleotide that is a functional fragment of an amino acid sequence of SEQ ID NO: 4, or a conservatively modified variant of the functional fragment of the amino acid sequence of SEQ ID NO: 4; wherein the polynucleotide encodes a polypeptide that directs the formation of normal joint structures. In some such aspects, the normal joint structures include cartilage, ligaments, and tendons. In other such aspects, the recombinant polynucleotide encodes a polypeptide comprising the sequence of SEQ ID NO: 4. In some such aspects, the recombinant polynucleotide comprises SEQ ID NO: 3 or its complement. In other such aspects, the recombinant polynucleotide comprises SEQ ID NO: 4 or its complement.

In another aspect, the invention provides vectors comprising the recombinant polynucleotide as described above.

In another aspect, the invention provides an expression vector comprising the recombinant polynucleotide of the invention operatively linked to a regulatory sequence that controls expression of the polynucleotide in a host cell. In some such expression vectors, the recombinant polynucleotide is operatively linked to the regulatory sequence in an antisense orientation. In other such expression vectors, the recombinant polynucleotide is operatively linked to the regulatory sequence in a sense orientation.

In another aspect, the invention provides a host cell comprising the recombinant polynucleotide as described above or progeny of the cell. In some such aspects, the host cell is a prokaryote. In other such aspects, the host cell is a eukaryote.

In another aspect, the invention provides a host cell comprising the recombinant polynucleotide as described above operatively linked with a regulatory sequence that controls expression of the polynucleotide in a host cell. In some such aspects, the nucleic acid is operatively linked to the regulatory sequence in an antisense orientation. In other such aspects, wherein the nucleic acid is operatively linked to the regulatory sequence in a sense orientation.

In another aspect the invention provides an isolated DNA that encodes a hCDMP-1 protein variant as shown in SEQ ID NO: 4.

In another aspect, the invention provides an antisense oligonucleotide complementary to a messenger RNA comprising SEQ ID NO: 3 and encoding a hCDMP-1 variant or homolog thereof, wherein the oligonucleotide inhibits the expression of hCDMP-1.

In another aspect, the invention provides the recombinant polynucleotide of the invention that is RNA.

In another aspect, the invention provides a method of producing a polypeptide comprising: (i) culturing the host cell comprising the recombinant polynucleotide of as described above operatively linked with a regulatory sequence that controls expression of the polynucleotide in a host cell under conditions such that the polypeptide is expressed; and (ii) recovering the polypeptide from the cultured host cell of its cultured medium.

In another aspect, the invention provides a polypeptide encoded by (a) a polynucleotide that has the sequence of SEQ ID NO: 3; (b) a polynucleotide that hybridizes under stringent hybridization conditions to (a) and encodes an amino acid sequence of SEQ ID NO: 4. In some such aspects, the amino acid sequence of SEQ ID NO: 4. In other such aspects, the polypeptide is soluble. In some such aspects, the polypeptide is fused with a heterologous peptide.

In another aspect, the invention provides a pharmaceutical composition comprising a polynucleotide of the invention, or a polypeptide encoded by (a) a polynucleotide that has the sequence of SEQ ID NO: 3; (b) a polynucleotide that hybridizes under stringent hybridization conditions to (a) and encodes an amino acid sequence of SEQ ID NO: 4, and a pharmaceutically acceptable carrier.

In another aspect, the invention provides a recombinant expression system comprising: the recombinant polynucleotide as described above.

In another aspect, the invention provides a recombinant expression system for endoproteolytic processing of a hCDMP-1 protein variant comprising: a) a first nucleotide sequence encoding a hCDMP-1 protein variant having the amino acid sequence as set forth in SEQ ID NO: 4 or conservative substitution thereof; b) a second nucleotide sequence encoding SPC1, and c) a third nucleotide sequence encoding SPC6; wherein the first, second and third nucleotide sequences are independently operatively linked to transcription controlling nucleotide sequences in a host cell. In some such expression systems, the host cell is an autologous cell. In other such expression systems, the host cell is an allogeneic cell. In some such expression systems, the host cell is a functional progenitor cell capable of differentiating into skeletal tissue. In other such expression systems, the host cell is a chondrocyte progenitor cell. In some such expression systems, the skeletal tissue is cartilage, bone, ligament, or tendon. In other such expression systems, the host cell is isolated from the synovium, periosteum, perichondrium, or other source of cells capable of differentiating into skeletal tissue.

In another aspect, the invention provides a method of modulating musculoskeletal disorders in a subject, the method comprising the step of administering to the subject a therapeutically effective amount of a nucleic acid encoding the recombinant polynucleotide of the invention as described above. In some such aspects, the method further comprises the step of administering to the subject a therapeutically effective amount of a second nucleic acid encoding SPC1 and a third nucleic acid encoding SPC6.

In another aspect, the invention provides a method of modulating musculoskeletal disorders in a subject, the method comprising the step of administering to the subject a therapeutically effective amount of a nucleic acid encoding a hCDMP-1 polypeptide variant, wherein the nucleic acid hybridizes under stringent conditions to a nucleic acid encoding a polypeptide having an amino acid sequence of SEQ ID NO: 4.

In another aspect, the invention provides a method for modulating musculoskeletal disorders in a subject comprising the steps of: (a) isolating cells to be implanted into said subject (b) introducing into the cells the recombinant expression system as described above; and (c) implanting the cells containing the recombinant expression system into said subject. In some such methods, the cells express wildtype hCDMP-1. In other such methods, the cells do not express wildtype hCDMP-1. In some such methods, the cells are functional progenitor cells. In some such methods, the functional progenitor cells are chondrocyte progenitor cells. In some such methods, the cells are isolated from the synovium, periosteum, perichondrium, or other source of cells capable of differentiating into skeletal tissue.

In another aspect, the invention provides a method for modulating musculoskeletal disorders in a subject in need thereof, comprising: (a) selecting the patient in need thereof; (b) isolating cells from the patient, wherein the cells express CDMP-1 and introducing into the cells a first nucleotide sequence encoding SPC1 and a second nucleotide sequence encoding SPC6, wherein the first and second nucleotide sequences are independently operatively linked to transcription controlling nucleotide sequences in the isolated cells; and (c) readministering the cells to the patient.

In another aspect, the invention provides a method for modulating musculoskeletal disorders in a subject in need thereof, comprising: (a) selecting the patient in need thereof, (b) isolating cells from the patient, wherein the cells do not express CDMP-1; and introducing into the cells the recombinant expression system as described above; and (c) readministering the cells to the patient. In some such methods, the cells are functional progenitor cells. In other such methods, the functional progenitor cells are chondrocyte progenitor cells. In some such methods, cells are isolated from the synovium, periosteum, perichondrium, or other source of cells capable of differentiating into skeletal tissue.

### BRIEF DESCRIPTION OF THE DRAWINGS

FIG. 1. Characterization and expression analysis of Xenopus GDF5. A. Dendrogram showing the phylogenetic relationship between members of the GDF5, 6, 7, and 16 subfamily. Full-length amino acid sequences were analyzed using GeneWorks® Version 2.2 (IntelliGenetics, Inc) software. B. Amino acid sequence comparison of full-length human, mouse, and Xenopus GDF5. Sequence identities are boxed; sequence similarities are highlighted in gray. C. RT-PCR analysis for Xenopus GDF5 using RNA obtained from indicated stages of Xenopus development (h=hindlimb, f=forelimb). D. Whole mount in situ hybridization of Xenopus GDF5 of a stage 59 Xenopus forelimb. Arrows indicate the location of positive signal at presumptive joint interzones.

FIG. 2. Comparison of BMP precursor cleavage sites and SPC temporal expression pattern. A. Amino acid sequences surrounding the consensus RXXR cleavage sites for various BMP precursors. The residues shown in color fit the required sequence for SPC cleavage and the boxed residues illustrate the sequence shared by CDMP1/GDF5 and Vg1. B. RT-PCR analysis of indicated Xenopus stages for RNA encoding different SPCs. C. Nucleotide and amino acid sequence of the full-length CDMP-1 cDNA. The predicted CDMP-1 product contains 500 amino acids with a putative proteolytic processing site (RXXR/A, box) preceding a 120-amino acid mature carboxyl-terminal region. A single N-linked glycosylation site is located in the pro-region (asterisk). The putative signal peptide (42) is underlined in bold. A termination codon (TGA) is shown in the 5′-untranslated region. A vertical arrowhead marks the boundary between sequence obtained from genomic DNA and cDNA (from Chang et al., J. Biol. Chem. 45: 28227-28234, 1994).

FIG. 3. Ventralizing activity observed two days following injection of dorsal blastomeres of Xenopus (two and four cell) embryos with mRNA encoding wild type or mutant GDF5 and combinations of various SPCs. A. Control, sham injected embryos B. Embryos injected with wild type GDF5 (100 pg) alone. C. Embryos injected with GDF5 K→R mutant (100 pg). D. Diagrammatic representation of experiments in which embryos were injected with GDF5-wt alone (60 pg), GDF5-wt (60 pg)+Furin (300 pg) or SPC6 (300 pg), GDF5-wt (60 pg)+Furin (150 pg)+SPC6 (150 pg) or GDF5 K→R mutant (60 pg). Injection with Furin (150 pg)+SPC6 (150 pg) only was included as a negative control. Embryos were assessed using the Dorso-Anterior Index (DAI) scale described by Kao and Elinson (Dev Biol. 1988 Can; 127, 64-77). The DAI of the embryos was scored after 2 days, and the average DAI for each sample is shown. Numbers of embryos examined (n) are indicated above each column. Embryos with a DAI of 0 lack dorsal structures completely and those with a DAI of 5 are normal. Similar results were obtained in three separate experiments. E. Immunoblot analysis of secreted proteins following mRNA injection of Xenopus oocytes. Xenopus oocytes (stage V1) were isolated, defolliculated, and injected with mRNAs encoding GDF5-T7 (25 ng), GDF5-T7 (25 ng)+Furin (25 ng), GDF5 (25 ng)+SPC6 (25 ng) or GDF5 (25 ng)+Furin (12.5 ng)+SPC6 (12.5 ng). Injected oocytes were incubated at 118° C. for 24 hours and oocyte supernatants were prepared for analysis as described in methods. Arrows indicate the locations of the pro- and mature forms of GDF5-T7. Similar results were obtained in three separate experiments.

FIG. 4. Animal cap experiments and RT-PCR analyses. Single dorsal blastomeres of Xenopus embryos at the four cell stage were injected with mRNAs for either Vg1-wt (100 pg)+GFP (200 pg), or Vg1-wt (100 pg)+Furin (200 pg), Vg1-wt+Furin (100 pg)+SPC4 (100 pg), Vg1-wt (100 pg)+Furin (100 pg)+SPC6 (100 pg), Vg1-wt+SPC4 (100 pg)+SPC6 (100 pg), or B-Vg1 (100 pg). GFP (300 pg) and Furin (150 pg)+SPC6 (150 pg) were used as negative controls (not shown). Animal caps, removed when the embryos reached stage 9, were cultured in 0.5×MMR until sibling, non-injected embryos reached stage 24. A. Morphology of representative animal caps from each treatment. B. RT-PCR analysis of animal cap explants for mesodermal markers. Histone H4 was analyzed to demonstrate that equivalent amounts of template were used in each amplification. Similar results were obtained in three separate experiments. C. Immunoblot analysis of secreted proteins following mRNA injection of Xenopus oocytes. Xenopus oocytes (stage V1) were isolated, defolliculated, and injected with mRNAs encoding Vg1-T7 (25 ng), Vg1-T7 (25 ng)+Furin (25 ng), Vg1-T7 (25 ng)+SPC6 (25 ng) or Vg1-T7 (25 ng)+Furin (12.5 ng)+SPC6 (12.5 ng). Injected oocytes were incubated at 18° C. for 24 hours and oocyte supernatants were prepared for analysis as described in methods. Arrows indicate the locations of the pro- and mature forms of Vg1-T7. Similar results were obtained in three separate experiments.

FIG. 5. Spatial distribution of GDF5 and SPCs in 15.5dpc mouse embryo limbs. Panels show GDF5 expression in red in the region of joint interzones. Furin and SPC6 expression are in green. The boxed areas are shown at higher magnification in adjacent panels. While Furin and SPC6 were distributed widely in the developing digit, their expression overlapped with that of GDF5, predominantly at the boundary of GDF5 expression (indicated by white arrows).

FIG. 6. Spatial distribution of Vg1 and SPCs in stage 8 Xenopus blastulae. Panels show Vg1 expression in red and Furin, SPC4, and SPC6 in green. The double-label images indicate the specific overlapping expression patterns of Vg1 with Furin, SPC4, and SPC6 (indicated by white arrows) on the dorsal-vegetal side of the embryo, in the region of the Nieuwkoop center. Adjacent sections were probed with Siamois to confirm dorsal-ventral orientation of the embryos (not shown).

FIG. 7. Vegetal expression of Furin and SPC6A can restore a normal dorsal axis to UV-ventralized Xenopus embryos. UV-treated embryos were injected in a vegetal blastomere at the 4 (B, D and E) or 8 (C) cell stage with 1 ng GFP (B) or 450 pg Furin and 450 pg SPC6A (C and D) mRNA, All embryos were allowed to develop until control non-UV-treated embryos (A) reached stage 37/38. Control injected embryos had DAIs of 0-2 (B) whereas Furin/SPC6A rescued embryos had DAIs ranging from 2-3 (C) to 4-5 (D) depending on the time of injection. The representative rescued embryos shown in D have anterior structures, including eyes, cement gland, and neural crest-derived pigmented cells. The distribution of DAI scores for a representative experiment is shown in (E). Experiments were performed six times in which 0/150 of control, UV irradiated embryos had DAI>2.

### DETAILED DESCRIPTION

### 1. Introduction

The invention provides a number of methods, reagents, and compounds that can be used for the treatment of musculoskeletal disorders. It is to be understood that this invention is not limited to particular methods, reagents, compounds, compositions, or biological systems, which can, of course, vary. It is also to be understood that the terminology used herein is for the purpose of describing particular embodiments only, and is not intended to be limiting. As used in this specification and the appended claims, the singular forms “a”, “an”, and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably +1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“TGF-β superfamily” refers to a family of structurally related growth factors, all of which possess physiologically important growth-regulatory and morphogenetic properties. This family of related growth factors is well known in the art (Kingsley et al., Genes Dev. 8: 133-46, 1994; and Hoodless et al., Curr. Topics Microbiol. Immunol. 228: 235-72, 1998). The TGF-β superfamily includes Bone Morphogenetic Proteins (BMPs), Activins, Inhibins, Mullerian Inhibiting Substance, Glial-Derived Neurotrophic Factor, and a still growing number of Growth and Differentiation Factors (GDFs), such as GDF-5.

“Bone morphogenetic protein” or “BMP” refers to a protein belonging to the BMP family of the TGF-β superfamily of proteins defined on the basis of DNA and amino acid sequence homology. According to this invention, a protein belongs to the BMP family when it has at least 50% (e.g., at least 70% or even 85%) amino acid sequence similarity or given identity with a known BMP family member within the conserved C-terminal cysteine-rich domain that characterizes the BMP family. Members of the BMP family can have less than 50% DNA or amino acid sequence similarity or identity overall. BMPs act to induce the differentiation of mesenchymal-type cells into chondrocytes and osteoblasts before initiating bone formation. Some BMPs act to induce differentiation of cartilage- and bone-forming cells near sites of fractures but also at ectopic locations. Some of the proteins induce the synthesis of alkaline phosphatase and collagen in osteoblasts. Some BMPs act directly on osteoblasts and promote their maturation while at the same time suppressing myogenous differentiation. Other BMPs promote the conversion of mesecnhemyl cells into chondrocytes and are capable also of inducing the expression of an osteoblast phenotype in non-osteogenic cell types.

As used herein, “bone morphogenetic protein-14”, “BMP-14”, “BMP14” “cartilage-derived morphogenetic protein-1”, “CDMP-1”, “CDMP1,” “growth differentiation factor 5”, “GDF-5”, “GDF5”, “GDF5 precursor” are used interchangeably. The NCBI Accession Number for GDF5 includes AAA57007, AAH32495 and AB019005 with a NCBI RefSeq of NP_{—}000548, OMIM number of 601146, and UniProt number of P43026 and Q96SB1 (see, e.g., Chang et al., J. Biol. Chem. 269: 28227-28234, 1994; Paralkar et al., J. Biol. Chem. 273: 13760-13767, 1998; Tomaski et al., Arch Otolaryngol Head Neck Surg. 125: 901-906, 1999). BMP-14/CDMP-1/GDF-5 is a 501 amino acid precursor protein with a 121 amino acid mature chain, (chromosome 20q11.2). Therefore, BMP-14/CDMP-1/GDF-5 refer to the full length protein or BMP-14/CDMP-1/GDF-5 precursor protein. BMP-14/CDMP-1/GDF-5 also refers to any “mature” protein, functional fragment or conservatively modified variant of a functional fragment thereof. BMP-14/CDMP-1/GDF-5 is predominantly expressed in the interzones of developing joints during embryonic development; it is involved in bone formation. Defects in BMP14/GDF5/CDMP1 can cause Acromesomelic chondrodysplasia (short forearms, hands and feet characterize this form of dwarfism). As used herein, “BMP”, “BMP polypeptide”, or “BMP of the invention” and “BMPs of the invention” are used interchangeably. Similarly, “BMP-14”, “BMP-14 polypeptide”, or “BMP-14 of the invention” and “BMP-14s of the invention” are used interchangeably. Similarly “GDF-5”, or GDF-5 polypeptide, or “GDF-5 of the invention” and “GDF-5s of the invention” are used interchangeably. In addition, “CDMP-1”, “CDMP-1”, “CDMP-1 polypeptide”, or “CDMP-1 of the invention” and “CDMP-1s of the invention” are used interchangeably.

As used herein, “serine endopeptidases” and “serine proteases” are used interchangeably. Serine endopeptidases are any member of a group of peptidases, which are characterized by the presence of a serine residue in the active site of the enzyme. These proteins mediate limited proteolytic cleavage of a number of molecules, for example, growth factors, prohormones, proneuropeptides, zymogens, and adhesion molecules, and influence a variety of fundamental cellular functions. A subset of serine endopeptidases, proprotein convertases (PCs), cleave proproteins, to their active fragments by endoproteolytic processing through limited proteolysis at one or at most two specific cleavage sites. PCs can regulate the activation of bone growth factors during embryonic development. In eukaryotes, these enzymes are also called “subtilisin-like proprotein convertases” or SPC's. Most SPC's are autocatalytic and must be activated by cleavage of their propeptide before they can cleave their specific substrates. The conversion of a precursor protein to an active protein usually occurs by cleavage at basic motifs (e.g., R-X-X-R, where X can be any amino acid). For a review of proprotein convertases, see, e.g., Taylor et al., FASEB J. 17: 1215-1227, 2003.

As used herein, “proprotein convertase subtilisin/kexin type 6”, “PCSK-6”, “PCSK6”, “subtilisin/kexin-like protease PACE4”, “PACE4”, “paired basic amino acid cleaving enzyme 4”, “subtilisin-like proprotein convertase-4”, “SPC-4”, and “SPC4” are used interchangeably. The NCBI Accession Number for SPC4 includes AAA59998, AB001898, and AB001905 with a NCBI Ref Seq NP_{—}002561, OMIM number of 167405, and UniProt numbers of P29122, Q15099, and Q15100. Similarly, “proprotein convertase subtilisin/kexin type 5 precursor”, “proprotein convertase PC5, “proprotein convertase subtilisin/kexin type 5”, “PCSK-5”, “PCSK5”, “subtilisin/kexin-like protease PC5”, “subtilisin/kexin-like proprotein-6”, “SPC-6”, and “SPC6” are used interchangeably. The NCBI Accession Number for SPC6 includes AAA91807, AAC50643, and AAH12064 with a NCBI Ref Seq NP_{—}006191, OMIM number of 600488, and UniProt numbers of Q3527 and Q92824. Similarly, “furin”, “furin precursor”, “dibasic processing enzyme”, “FUR”, “furin (paired basic amino acid cleaving enzyme)”, “PACE”, “paired basic amino acid residue cleaving enzyme”, “PCSK3”, “SPC1” are used interchangeably. The NCBI Accession Number for SPC6 includes AAB28140, AAH12181, and BC008295 with a NCBI Ref Seq NP_{—}002560, OMIM number of 136950, and UniProt numbers of P09958 and Q14336.

As used herein, “vegetal hemisphere VG1 Protein”, “Vg1”, “VG1” and “DVR-1 protein precursor” are used interchangeably. “Vg1” is an activin-like protein concentrated in the vegetal hemisphere of the Xenopus (frog) embryo and is a growth factor related to TGF-β. The NCBI Accession Number for vg1 includes P09543, and cross reference dbsource designations of M18055.1, AAA49727.1 and A29619 (see, e.g., Weeks and Melton, Cell 51: 861-867, 1987; and Dale, EMBO J. 8: 1057-1065, 1989).

Unless specifically referred to, the phrase “human BMP (hBMP)”, as used herein refers to “hBMP”. Unless specifically referred to, the phrase “human GDF-5 (hGDF5)”, as used herein refers to “hGDF5”. Unless specifically referred to, the phrase “human CDMP-1 (hCDMP-1)” as used herein refers to “hCDMP-1”. The phrase “human CDMP-1 variant or homolog” refers to a polynucleotide that has the sequence of SEQ ID NO: 3; (b) a polynucleotide that hybridizes under stringent hybridization conditions to (a) and encodes an amino acid sequence of SEQ ID NO: 4; or (c) a polynucleotide that is a functional fragment of an amino acid sequence of SEQ ID NO: 4, or a conservatively modified variant of the functional fragment of the amino acid sequence of SEQ ID NO: 4; wherein the polynucleotide encodes a polypeptide that directs the formation of normal joint structures including but not limited to cartilage, ligaments, and tendons.

“Morphogenesis protein” refers to a protein having morphogenesis activity. For instance, this protein is capable of inducing progenitor cells to proliferate and/or to initiate differentiation pathways that lead to the formation of cartilage, bone, tendon, ligament, neural or other types of tissue, depending on local environmental cues. Thus, morphogenesis proteins useful in this invention can behave differently in different surroundings. A morphogenesis protein of the invention can comprise at least one polypeptide belonging to the BMP family. A preferred morphogenesis protein of the invention includes a hCDMP-1 variant comprising the sequence of SEQ ID NO: 4.

“Morphogenesis activity,” “inducing activity” and “tissue inductive activity” alternatively refer to the ability of an agent to stimulate a target cell to undergo one or more cell divisions (proliferation) that can optionally lead to cell differentiation. Such target cells are referred to generically herein as progenitor cells. Cell proliferation is typically characterized by changes in cell cycle regulation and can be detected by a number of means which include measuring DNA synthetic or cellular growth rates, changes in messenger RNA profiles, changes in phosphorylation states or other characteristics associated with the status of signal transduction machinery within the cell. Early stages of cell differentiation are typically characterized by changes in gene expression patterns relative to those of the progenitor cell; such changes can be indicative of a commitment towards a particular cell fate or cell type. Later stages of cell differentiation can be characterized by changes in gene expression patterns, cell physiology, and morphology. Any reproducible change in gene expression, cell physiology, or morphology can be used to assess the initiation, nature and extent of cell differentiation induced by a morphogenic protein.

Stem cells are undifferentiated cells defined by their ability at the single cell level to both self-renew and differentiate to produce progeny cells, including self-renewing progenitors, non-renewing progenitors and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent—able to give rise to all embryonic and extraembryonic cell types; (2) pluripotent—able to give rise to all embryonic cell types; (3) multipotent—able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood); (4) oligopotent—able to give rise to a more restricted subset of cell lineages than multipotent stem cells; and (5) unipotent—able to give rise to a single cell lineage (e.g., spermatogenic stem cells).

Stem cells are also categorized on the basis of the source from which they can be obtained. An adult stem cell is generally a multipotent undifferentiated cell found in tissue comprising multiple differentiated cell types. The adult stem cell can renew itself and, under normal circumstances, differentiate to yield the specialized cell types of the tissue from which it originated, and possibly other tissue types. An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the placenta and the umbilical cord. These cells have been found to possess features characteristic of pluripotent stem cells, including rapid proliferation and the potential for differentiation into many cell lineages. Postpartum stem cells can be blood-derived (e.g., as are those obtained from umbilical cord blood) or non-blood-derived (e.g., as obtained from the non-blood tissues of the umbilical cord and placenta).

Embryonic tissue is typically defined as tissue originating from the embryo (which in humans refers to the period from fertilization to about six weeks of development. Fetal tissue refers to tissue originating from the fetus, which in humans refers to the period from about six weeks of development to parturition. Extraembryonic tissue is tissue associated with, but not originating from, the embryo or fetus. Extraembryonic tissues include extraembryonic membranes (chorion, amnion, yolk sac and allantois), umbilical cord, and placenta (which itself forms from the chorion and the maternal decidua basalis).

Differentiation is the process by which an unspecialized (“uncommitted”) or less specialized cell acquires the features of a specialized cell, such as a nerve cell or a muscle cell, for example. A differentiated or differentiation-induced cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the origin of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

In a broad sense, a progenitor cell is a cell that has the capacity to create progeny cells that are more differentiated than itself and yet retain the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell can be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

A “chondrocyte progenitor cell,” as used herein, refers to a pluripotent, or lineage-uncommitted, progenitor cell that is potentially capable of an unlimited number of mitotic divisions to either renew its line or to produce progeny cells that will differentiate into chondrocytes. This cell is typically referred to as a “stem cell” or “mesenchymal stem cell” in the art. Alternatively, a “chondrocyte progenitor cell” is a lineage-committed progenitor cell produced from the mitotic division of a stem cell that will eventually differentiate into a chondrocyte. The lineage-committed progenitor cell is generally incapable of an unlimited number of mitotic divisions and will eventually differentiate into a chondrocyte. Chondrocyte progenitor cells can come from the synovium or bone marrow, if the subchondral bone plate is penetrated, or other tissues.

“Skeletal tissue” includes cartilage, bone, ligament, or tendon.

Unless defined otherwise, “cartilage,” “bone,” “ligaments,” “tendons,” “synovium,” “periosteum,” “perichondrium” and related words have their standard meaning. See, e.g., http://www.stedmans.com/, http://www.m-w.com/, http://www.medlineplus.gov/ and other references cited below.

“Cartilage” refers to elastic, translucent connective tissue in mammals, including human and other species. See, e.g., http://www.biologydaily.com/dictionary/. Cartilage is composed predominantly of chondrocytes, type II collagen, small amounts of other collagen types, other noncollagenous proteins, proteoglycans, and water, and is usually surrounded by a perichondrium, made up of fibroblasts, in a matrix of type I and type II collagen as well as other proteoglycans. Although most cartilage becomes bone upon maturation, some cartilage remains in its original form in locations such as the joints, nose, ears, knees, and between intervertebral disks. Cartilage as no blood or nerve supply and chondrocytes are the only type of cell in this tissue.

The function of bone is to provide mechanical support for joints, tendons and ligaments, to protect vital organs from damage. Bone cells include osteoblasts, the so-called Bone Lining Cells (BLCs), osteocytes, osteoclasts, and other cell types (see http://www.biologydaily.com/biology/Bone). Osteoblasts are typically viewed as bone forming cells. They are located near to the surface of bone and their functions are to make osteoid and manufacture hormones such as prostaglandins that act on bone itself. Osteoblasts are mononucleate. Active osteoblasts are situated on the surface of osteoid seams and communicate with each other via gap-junctions. Bone Lining Cells (BLCs) share a common lineage with osteogenesis (bone forming) cells. They are flattened, mononucleate cells which line bone. Osteocytes originate from osteoblasts that have migrated into and become trapped and surrounded by bone matrix, which they themselves produce. The spaces that they occupy is known as lacunae. Osteocytes have many processes, which reach out to meet osteoblasts, probably for the purposes of communication. Their functions include formation of bone, matrix maintenance, and calcium homeostasis. Osteocytes possibly act as mechano-sensory receptors—regulating the bone's response to mechanical stress. For a complete overview on bone biology incorporating the description above, see http://www.biologydaily.com/biology/Bone.

Tissues connecting bones and muscles are collectively referred to as “connective tissues.” Ligaments are short bands of tough fibrous connective tissue composed mainly of long, stringy collagen molecules. See, for example, http://www.biologydaily.com/dictionary/. Ligaments generally connect bones to other bones in joints. Tendons are fibrous connective tissues, attached on one end to a muscle and on the other to a bone. The “synovium” or “synovial membrane” is a thin layer of tissue that lines the non-cartilaginous surfaces within the joint space, sealing it from the surrounding tissue. See, e.g., http://www.biologydaily.com/dictionary/. The membrane contains a fibrous outer layer, as well as an inner layer that is responsible for the production of specific components of synovial fluid, which nourishes and lubricates the joint. By “synovial cells” is meant cells derived from the synovium. “Periosteum” refers to the membrane of fibrous connective tissue that closely invests all bones except at the articular surfaces. See, e.g., http://www.biologydaily.com/dictionary/. By “periosteal cells” is meant cells derived exclusively from the periosteum. Periosteal cells can be separated from the periosteum by well-known techniques in the art; subjecting periosteal tissue to trypsinization is but one of many examples for obtaining periosteal cells. The cells, once released from the periosteum or periosteal tissue, can then be grown in cell culture. “Perichondrium” refers to the membrane of connective tissue covering the surface of cartilage except at the articular surfaces. See, for example, http://www.biologydaily.com/dictionary/. The perichondrium nourishes the avascular cartilage, and it also contains cells including mesenchymal cells, which can differentiate into chondroblasts.

“Cell culture” refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times can be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there can be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, and time between passaging.

A conditioned medium is a medium in which a specific cell or population of cells has been cultured, and then removed. While the cells are cultured in the medium, they secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

Generally, a trophic factor is defined as a substance that promotes survival, growth, proliferation, maintenance, differentiation, and/or maturation of a cell, or stimulates increased activity of a cell.

“Standard growth conditions”, as used herein, refers to culturing of cells (e.g., mammalian cells) at 37° C., in a standard atmosphere comprising 5% CO₂. Relative humidity is maintained at about 100%. While the foregoing the conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like. For example, “standard growth conditions” for yeast (e.g., S. cerevisiae) include 30° C. and generally under regular atmospheric conditions (less than 0.5% CO₂, approximately 20% O₂, approximately 80% N₂) at a relative humidity at about 100%.

“Gene” refers to a unit of inheritable genetic material found in a chromosome, such as in a human chromosome. Each gene is composed of a linear chain of deoxyribonucleotides, which can be referred to by the sequence of nucleotides forming the chain. Thus, “sequence” is used to indicate both the ordered listing of the nucleotides that form the chain, and the chain that has that sequence of nucleotides. The term “sequence” is used in the same way in referring to RNA chains, linear chains made of ribonucleotides. The gene includes regulatory and control sequences, sequences that can be transcribed into an RNA molecule, and can contain sequences with unknown function. Some of the RNA products (products of transcription from DNA) are messenger RNAs (mRNAs), which initially include ribonucleotide sequences (or sequence) that are translated into a polypeptide and ribonucleotide sequences that are not translated. The sequences that are not translated include control sequences, introns, and sequences with unknowns function. It can be recognized that small differences in nucleotide sequence for the same gene can exist between different persons, or between normal cells and cancerous cells, without altering the identity of the gene.

“Isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19: 5081, 1991; Ohtsuka et al., J. Biol. Chem. 260: 2605-2608, 1985); and Cassol et al., 1992; Rossolini et al., Mol. Cell. Probes 8: 91-98, 1994). For arginine and leucine, modifications at the second base can also be conservative. The term nucleic acid is used interchangeably with gene, cDNA, and mRNA encoded by a gene.

As used herein a “nucleic acid probe” is defined as a nucleic acid capable of binding to a target nucleic acid (e.g., a nucleic acid associated with cancer) of complementary sequence through one or more types of chemical bonds, usually through complementary base pairing, usually through hydrogen bond formation. As used herein, a probe can include natural (i.e., A, G, C, or T) or modified bases (7-deazaguanosine, inosine, and the like). In addition, the bases in a probe can be joined by a linkage other than a phosphodiester bond, so long as it does not interfere with hybridization. Thus, for example, probes can be peptide nucleic acids in which the constituent bases are joined by peptide bonds rather than phosphodiester linkages. It will be understood by one of skill in the art that probes can bind target sequences lacking complete complementarity with the probe sequence depending upon the stringency of the hybridization conditions.

Nucleic acid probes can be DNA or RNA fragments. DNA fragments can be prepared, for example, by digesting plasmid DNA, or by use of PCR, or synthesized by either the phosphoramidite method described by Beaucage and Carruthers, Tetrahedron Lett. 22: 1859-1862, 1981) (Beaucage and Carruthers), or by the triester method according to Matteucci et al., J. Am. Chem. Soc. 103: 3185, 1981) (Matteucci), both incorporated herein by reference. A double stranded fragment can then be obtained, if desired, by annealing the chemically synthesized single strands together under appropriate conditions, or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

A “labeled nucleic acid probe” is a nucleic acid probe that is bound, either covalently, through a linker, or through ionic, van der Waals, or hydrogen bonds to a label such that the presence of the probe can be detected by detecting the presence of the label bound to the probe.

The phrase “a nucleic acid sequence encoding” refers to a nucleic acid that contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons that encode a single amino acid) of the native sequence or sequences that can be introduced to conform with codon preference in a specific host cell.

“Polypeptides of the invention,” “polynucleotides of the invention,” includes all polypeptides and polynucleotides described below (e.g., see Figures).

Polynucleotides of the present invention can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

Polypeptides can be composed of amino acids joined to each other by peptide bonds or modified peptide bonds, i.e., peptide isosteres, and can contain amino acids other than the 20 gene-encoded amino acids. The polypeptides can be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification can be present in the same or varying degrees at several sites in a given polypeptide. Also, a given polypeptide can contain many types of modifications. Polypeptides can be branched, for example, as a result of ubiquitination, and they can be cyclic, with or without branching. Cyclic, branched, and branched cyclic polypeptides can result from posttranslation natural processes or can be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2^{nd} Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al, Meth Enzymol 182: 626-646, 1990; Rattan et al., Ann N.Y. Acad Sci 663: 48-62, 1992).

Polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

Polypeptides can be in the form of the secreted protein, including the mature form, or can be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

Polypeptides are preferably provided in an isolated form, and preferably are substantially purified. A recombinantly produced version of a polypeptide, including the secreted polypeptide, can be substantially purified using techniques described herein or otherwise known in the art, such as, for example, by the one-step method described in Smith and Johnson, Gene 67: 31-40, 1988. Polypeptides of the invention also can be purified from natural, synthetic, or recombinant sources using techniques described herein or otherwise known in the art, such as, for example, antibodies of the invention raised against the polypeptides of the present invention using methods well known in the art.

“Ortholog” refers to an evolutionarily conserved bio-molecule represented in a species other than the organism in which a reference sequence is identified, and contains a nucleic-acid or amino-acid sequence that is homologous to the reference sequence. To determine the degree of similarity between a reference sequence and a sequence in question, two nucleic-acid sequences or two amino-acid sequences are compared. Homology can be defined by testing percentage identity or percentage similarity similarity for statistical significance. Percentage identity correlates with the proportion of identical amino-acid residues shared between two sequences compared in an alignment. Percentage similarity correlates with the proportion of amino-acid residues having similar structural properties that is shared between two sequences compared in an alignment. Percentages of similarity and identity can be calculated over a portion of the primary structure and not over the entire gene/protein sequence. For example, amino-acid residues having similar structural properties can be substituted for one another, such as the substitutions of analogous hydrophilic amino-acid residues, and the substitution of analogous hydrophobic amino-acid residues. Percentages of similarity and identity can be calculated over a portion of the primary structure and not over the entire gene/protein sequence. For the present disclosure, an ortholog or an orthologous sequence is defined as a homologous molecule or a sequence that directs the formation of normal joint structures including but not limited to cartilage, ligaments, and tendons and a sequence identity of at least about 20%, 25%, 30%, 35%, 40%, 45%, 50%, 55%, 60%, 65%, 70%, 75%, 80%, 85%, 90%, and 95%. Alternatively, an ortholog is defined as a homologous molecule or sequence that directs the formation of normal joint structures including but not limited to cartilage, ligaments, and tendons and a sequence similarity of at least about 40%, 40%, 45%, 50%, 55%, 60%, 65%, 70%, 75%, 80%, 85%, 90%, and 95%.

It is further contemplated that “ortholog” is a polypeptide or nucleic acid molecule of an organism that is highly related to a reference protein, or nucleic acid sequence, from another organism. An ortholog is functionally related to the reference gene, protein or nucleic acid sequence. In other words, the ortholog and its reference molecule would be expected to fulfill similar, if not equivalent, functional roles in their respective organisms. It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Such functionally important domains can be defined by genetic mutations or by structure-function assays. Orthologs can be identified using methods provided herein. The functional role of an ortholog can be assayed using methods well known to the skilled artisan, and described herein. For example, function might be assayed in vivo or in vitro using a biochemical, immunological, or enzymatic assay; transformation rescue, or for example, in a nematode bioassay for the effect of gene inactivation on nematode phenotype. Alternatively, bioassays can be carried out in tissue culture; function can also be assayed by gene inactivation (e.g., by RNAi, siRNA, or gene knockout), or gene over-expression, as well as by other methods.

“Paralogs” are distinct but structurally related proteins made by an organism. Paralogs are believed to arise through gene duplication.

“Variant” can refer to an organism with a particular genotype in singular form, a set of organisms with different genotypes in plural form, and also to alleles of any gene identifiable by methods of the present invention. For example, the term “variants” includes various alleles that can occur at high frequency at a polymorphic locus, and includes organisms containing such allelic variants. The term “variant” includes various “strains” and various “mutants.”

A “wild type protein of the invention” or “native protein” of the invention comprises a polypeptide having the same amino acid sequence as a protein derived from nature. Thus, a wild type protein can have the amino acid sequence of a naturally occurring rat protein, murine protein, human protein, or protein from any other mammalian species. Such wild type GDF5/CDMP-1 polypeptides and orthologs thereof can be isolated from nature or can be produced by recombinant or synthetic means. The term “wild type protein” specifically encompasses naturally-occurring truncated forms of the protein, naturally-occurring variant forms (e.g., alternatively spliced forms), and naturally-occurring allelic variants of the particular proteins disclosed herein.

“Naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

An intact “antibody” comprises at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) through cellular receptors such as Fc receptors (e.g., FcγRI, FcγRIa, FcγRIIb, FcγRIII, and FcRη) and the first component (Clq) of the classical complement system. The term antibody includes antigen-binding portions of an intact antibody that retain capacity to bind the antigen. Examples of antigen binding portions include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341: 544-546, 1989), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al., Science 242: 423-426, 1988; and Huston et al., Proc. Natl. Acad. Sci. U.S.A. 85: 5879-5883, 1988). Such single chain antibodies are included by reference to the term “antibody” Fragments can be prepared by recombinant techniques or enzymatic or chemical cleavage of intact antibodies.

“Substantially pure” or “isolated” means an object species (e.g., an antibody of the invention) has been identified and separated and/or recovered from a component of its natural environment such that the object species is the predominant species present (i.e., on a molar basis it is more abundant than any other individual species in the composition); a “substantially pure” or “isolated” composition also means where the object species comprises at least about 50 percent (on a molar basis) of all macromolecular species present. A substantially pure or isolated composition can also comprise more than about 80 to 90 percent by weight of all macromolecular species present in the composition. An isolated object species (e.g., antibodies of the invention) can also be purified to essential homogeneity (contaminant species cannot be detected in the composition by conventional detection methods) wherein the composition consists essentially of derivatives of a single macromolecular species. For example, an isolated antibody to any one morphogenic gene product as contemplated herein can be substantially free of other antibodies that lack binding to that particular gene product and bind to a different antigen. Further, an isolated antibody that specifically binds to an epitope, isoform or variant of a protein of the invention, can, however, have cross-reactivity to other related antigens, e.g., from other species (e.g., GDF5/CDMP-1, SPC1, or SPC6 species homologs). Moreover, an isolated antibody of the invention be substantially free of other cellular material (e.g., non-immunoglobulin associated proteins) and/or chemicals.

“Specific binding” refers to preferential binding of an antibody to a specified antigen relative to other non-specified antigens. The phrase “specifically (or selectively) binds” to an antibody refers to a binding reaction that is determinative of the presence of the protein in a heterogeneous population of proteins and other biologics. Typically, the antibody binds with an association constant (Kₐ) of at least about 1×10⁶ M⁻¹ or 10⁷ M⁻¹, or about 10⁸ M⁻¹ to 10⁹ M⁻¹, or about 10¹⁰ M⁻¹ to 10¹¹ M⁻¹ or higher, and binds to the specified antigen with an affinity that is at least two-fold greater than its affinity for binding to a non-specific antigen (e.g., BSA, casein) other than the specified antigen or a closely-related antigen. The phrases “an antibody recognizing an antigen” and “an antibody specific for an antigen” are used interchangeably herein with the term “an antibody which binds specifically to an antigen”. A predetermined antigen is an antigen that is chosen prior to the selection of an antibody that binds to that antigen.

“Specifically bind(s)” or “bind(s) specifically”, when referring to a peptide, refers to a peptide molecule that has intermediate or high binding affinity, exclusively or predominately, to a target molecule. The phrase “specifically binds to” refers to a binding reaction that is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA, immunoprecipitation, Biacore, and Western blot are used to identify peptides that specifically react with the antigen. Typically a specific or selective reaction will be at least twice background signal or noise and more typically more than 10 times background.

“Substantially identical,” in the context of two nucleic acids or polypeptides refers to two or more sequences or subsequences that have at least about 80%, about 90%, about 95% or higher nucleotide or amino acid residue identity, when compared and aligned for maximum correspondence, as measured using the following sequence comparison method and/or by visual inspection. Such “substantially identical” sequences are typically considered to be homologous. The “substantial identity” can exist over a region of sequence that is at least about 50 residues in length, over a region of at least about 100 residues, over a region of at least about 150 residues, or over the full length of the two sequences to be compared. As described below, any two antibody sequences can only be aligned in one way, by using the numbering scheme in Kabat. Therefore, for antibodies, percent identity has a unique and well-defined meaning.

Amino acids from the variable regions of the mature heavy and light chains of immunoglobulins are designated Hx and Lx respectively, where x is a number designating the position of an amino acid according to the scheme of Kabat, Sequences of Proteins of Immunological Interest (National Institutes of Health, Bethesda, Md., 1987 and 1991). Kabat lists many amino acid sequences for antibodies for each subgroup, and lists the most commonly occurring amino acid for each residue position in that subgroup to generate a consensus sequence. Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Likewise, nucleic acids encoding antibody chains are aligned when the amino acid sequences encoded by the respective nucleic acids are aligned according to the Kabat numbering convention. An alternative structural definition has been proposed by Chothia, et al., J. Mol. Biol. 196: 901-917, 1987; Chothia, et al., Nature 342: 878-883, 1989; and Chothia, et al., J. Mol. Biol. 186: 651-663, 1989, which are herein incorporated by reference for all purposes.

The nucleic acids of the invention may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis, and others well known in the art (See, e.g., Sambrook, Tijssen, and Ausubel discussed herein and incorporated by reference for all purposes). The nucleic acid sequences of the invention and other nucleic acids used to practice this invention, whether RNA, cDNA, genomic DNA, or hybrids thereof, can be isolated from a variety of sources, genetically engineered, amplified, and/or expressed recombinantly. Any recombinant expression system can be used, including, in addition to bacterial, e.g., yeast, insect, or mammalian systems.

Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, see, e.g., Sambrook, Tijssen, and Ausubel. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The invention provides a recombinant expression system for endoproteolytic processing of a hCDMP-1 protein variant comprising: a) a first nucleotide sequence encoding a hCDMP-1 protein variant having the amino acid sequence as set forth in SEQ ID NO: 4 or conservative substitution thereof; b) a second nucleotide sequence encoding SPC1; and c) a third nucleotide sequence encoding SPC6; wherein the first, second and third nucleotide sequences are independently operatively linked to transcription controlling nucleotide sequences in a host cell.

The nucleic acid compositions of the present invention, while often in a native sequence (except for modified restriction sites and the like), from either cDNA, genomic or mixtures can be mutated, thereof in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Recombinant host cell” (or simply “host cell”) refers to a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny can not, in fact, be identical to the parent cell, but are still included within the scope of the term “host cell” as used herein.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A point mutation is one aspect of the invention, that, as discussed above, is conservative (i.e., a lysine to arginine change).

It is known in the art that one or more amino acids can be deleted from the N-terminus or C-terminus without substantial loss of biological function. See, e.g., Ron, et al., Biol. Chem., 268: 2984-2988, 1993. Accordingly, the present invention provides polypeptides having one or more residues deleted from the amino terminus. Similarly, many examples of biologically functional C-terminal deletion mutants are known (see, e.g., Dobeli, et al., 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above are included in the present invention. Thus, the invention further includes variations of the polypeptides which show substantial CDMP-1 polypeptide activity. Such mutants include deletions, insertions, inversions, repeats, and substitutions selected according to general rules known in the art so as to have little effect on activity.

There are two main approaches for studying the tolerance of an amino acid sequence to change, see, Bowie, et al., Science, 247: 1306-1310, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Examples include domains with enzymatic activity, e.g., a kinase domain. Typical domains are made up of sections of lesser organization such as stretches of β-sheet and α-helices. “Tertiary structure” refers to the complete three-dimensional structure of a polypeptide monomer. “Quaternary structure” refers to the three dimensional structure formed by the noncovalent association of independent tertiary units.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative spilling of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are contemplated here.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

“Biological samples” refers to any tissue or liquid sample having genomic DNA or other nucleic acids (e.g., mRNA) or proteins. It refers to samples of cells with a normal complement of chromosomes as well as samples of cells suspected of malignancy.

“Patient”, “subject”, or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a disease, condition or disorder as described herein. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a disease or disorder associated with a disease, condition or disorder as described herein, but does not yet experience or exhibit symptoms, inhibiting the symptoms of a disease or disorder (slowing or arresting its development), providing relief from the symptoms or side-effects of a disease (including palliative treatment), and relieving the symptoms of a disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

“Concomitant administration” of a known drug with a compound of the present invention means administration of the drug and the compound at such time that both the known drug and the compound will have a therapeutic effect or diagnostic effect. Such concomitant administration can involve concurrent (i.e., at the same time), prior, or subsequent administration of the drug with respect to the administration of a compound of the present invention. A person of ordinary skill in the art would have no difficulty determining the appropriate timing, sequence, and dosages of administration for particular drugs and compounds of the present invention.

In general, the phrase “well tolerated” refers to the absence of adverse changes in health status that occur as a result of the treatment and would affect treatment decisions.

“Synergistic interaction” refers to an interaction in which the combined effect of two or more agents is greater than the algebraic sum of their individual effects.

“Chronic” administration refers to administration of the agent(s) in a continuous mode as opposed to an acute mode, so as to maintain the initial therapeutic effect (activity) for an extended period of time. “Intermittent” administration is treatment that is not consecutive without interruption, but rather is cyclic in nature.

“Administering”, “introducing,” “delivering,” “placement,” and “transplanting” are used interchangeably herein and refer to the placement of cells of the invention into a subject by a method or route which results in at least partial localization of the regenerative cells at a desired site. The cells can be administered by any appropriate route that results in delivery to a desired location in the subject where at least a portion of the cells or components of the cells remain viable. The period of viability of the cells after administration to a subject can be as short as a few hours, e.g., twenty-four hours, to a few days, to as long as several years.

Several terms are used herein with respect to therapeutic applications described herein, e.g., cell replacement therapy. “Autologous transfer”, “autologous transplantation”, “autograft” and the like refer to treatments wherein the cell donor is also the recipient of the cell replacement therapy. “Allogeneic transfer”, “allogeneic transplantation”, “allograft” and the like refer to treatments wherein the cell donor is of the same species as the recipient of the cell replacement therapy, but is not the same individual. A cell transfer in which the donor's cells have been histocompatibly matched with a recipient is sometimes referred to as a “syngeneic transfer.” A syngeneic transfer is a special case of matched histocompatibility antigens, in which other antigens are matched by virtue of identical genetic makeup of donor and recipient owing to inbreeding. “Xenogeneic transfer”, “xenogeneic transplantation”, “xenograft” and the like refer to treatments wherein the cell donor is of a different species than the recipient of the cell replacement therapy.

As used herein, the phrase “musculoskeletal disorder” is intended to include all disorders related to bone, muscle, ligaments, tendons, cartilage and joints. For a review of musculoskeletal/connective tissue disorders, see Chapters 49-62 in THE MERCK MANUAL OF DIAGNOSIS AND THERAPY, 17^{th} Ed., Beers and Berkow, editors, 1999; this reference is herein incorporated by reference for all purposes). Treatment of a musculoskeletal disease or disorder is within the ambit of regenerative medicine. For example, disorders requiring spinal fixation, spinal stabilization, repair of segmental defects in the body (such as in long bones and flat bones), disorders of the vertebrae and discs including, but not limited to, disruption of the disc annulus such as annular fissures, chronic inflammation of the disc, localized disc herniations with contained or escaped extrusions, and relative instability of the vertebrae surrounding the disc are musculoskeletal disorders. Musculoskeletal disorders also include sprains, strains, and tears of ligaments, tendons, muscles and cartilage, tendonitis, tenosynovitis, fibromyalgia, osteoarthritis, rheumatoid arthritis, polymyalgia rheumatica, bursitis, acute and chronic back pain and osteoporosis, sports injuries and work related injuries including sprains, strains and tears of ligaments, tendons, muscles and cartilage, carpal tunnel syndrome, DeQuervains's disease, trigger finger, tennis elbow, rotator cuff, and ganglion cysts. In addition, musculoskeletal disorders include genetic diseases of the musculoskeletal system such as osteogenesis imperfecta, Duchenne, and other muscular dystrophies. Pain is the most common symptom and is frequently caused by injury or inflammation. Besides pain, other symptoms such as stiffness, tenderness, weakness, and swelling or deformity of affected parts are manifestations of musculoskeletal disorders. See also The National Institute of Arthritis and Musculoskeletal and Skin Dieseases, http://www.niams.nih.gov/.

“Cartilage disorder” refers to any injury or damage to cartilage, and to a collection of diseases that are manifested by symptoms of pain, stiffness, and/or limitation of motion of the affected body parts. Included within the scope of “cartilage disorders” is “degenerative cartilagenous disorders”, which is a collection of disorders characterized, at least in part, by degeneration or metabolic derangement of connective tissues of the body, including not only the joints or related structures, including muscles, bursae, synovial membrane, tendons, and fibrous tissue, but also the growth plate, meniscal system, and intervertebral discs.

“Degenerative cartilaginous disorders” includes “articular cartilage disorders,” which are characterized by disruption of the smooth articular cartilage surface and degradation of the cartilage matrix. Additional pathologies include nitric oxide production, and inhibition or reduction of matrix synthesis. Included within the scope of “articular cartilage disorder” are osteoarthritis (OA) and rheumatoid arthritis (RA). Examples of degenerative cartilagenous disorders include systemic lupus erythematosus and gout, amyloidosis or Felty's syndrome. Additionally, the term covers the cartilage degradation and destruction associated with psoriatic arthritis, kidney disorders, osteoarthrosis, acute inflammation (e.g., yersinia arthritis, pyrophosphate arthritis, gout arthritis (arthritis urica), and septic arthritis), arthritis associated with trauma, ulcerative colitis (e.g., Crohn's disease), multiple sclerosis, diabetes (e.g., insulin-dependent and non-insulin dependent), obesity, giant cell arthritis, and Sjogren's syndrome.

“Osteoarthritis” or “OA” defines not a single disorder, but the final common pathway of joint destruction resulting from multiple processes (see, for example, Hinton, Amer. Family Phys. 65: 841-848, 2002). OA is characterized by localized asymmetric destruction of the cartilage that when severe results in palpable bone enlargements at the joint margins. OA typically affects the interphalangeal joints of the hands, the first carpometacarpal joint, the hips, the knees, the spine, and some joints in the midfoot, while certain large joints, such as the ankles, elbows, and shoulders, tend to be spared. The knee and hip discussed above are large joints by any standards and are common sites of OA. OA can be associated with metabolic diseases such as hemochromatosis and alkaptonuria, developmental abnormalities such as developmental dysplasia of the hips (congenital dislocation of the hips), limb-length descrepancies, including trauma and inflammatory arthritides such as gout, septic arthritis, and neuropathic arthritis. OA can also develop after extended mechanical instability, such as resulting from sports injury or obesity. See also The Arthritis Foundation, http://www.arthritis.org/default.asp.

“Rheumatoid arthritis” or “RA” is a systemic, chronic, autoimmune disorder characterized by symmetrical synovitis of the joint and typically affects small and large diarthroid joints alike. See Newman, Understanding Rheumatoid Arthritis (Routledge UK 1996); for a review on the effects of rheumatoid arthritis on bone, see Haugeberg, Cur. Opin Rheumatol 15: 469-475, 2003. As RA progresses, symptoms can include fever, weight loss, thinning of the skin, multiorgan involvement, scleritis, corneal ulcers, the formation of subcutaneous or subperiosteal nodules, and even premature death. The symptoms of RA often appear during youth and can include vasculitis, atrophy of the skin and muscle, subcutaneous nodules, lymphadenopathy, splenomegaly, leukopaenia, and chronic anaemia. See also The Arthritis Foundation, http://www.arthritis.org/default.asp and http://www.mayoclinic.com/invoke.cfm?id=DS00020.

“Joint repair” refers to repair, reconstruction, or replacement of structures within a joint such as the articular surface (typically hyaline articular cartilage), ligaments, tendons, entheses, joint capsules, or synovial membranes to lessen pain and restore function associated with use of the joint in question.

“Inhibitors,” “activators,” and “modulators” of the molecules of the invention (genes their associated gene products in cells) are used to refer to inhibitory, activating, or modulating molecules, respectively, identified using in vitro and in vivo assays for binding or signaling, e.g., ligands, agonists, antagonists, and their homologs and mimetics. The term “modulator” includes inhibitors and activators. Inhibitors are agents that, e.g., bind to, partially or totally block stimulation, decrease, prevent, delay activation, inactivate, desensitize, or down regulate the activity of BMP or other genes, e.g., antagonists. Activators are agents that, e.g., bind to, stimulate, increase, open, activate, facilitate, enhance activation, sensitize or up regulate the activity of genes, e.g., agonists. Modulators include agents that, e.g., alter the interaction of gene or gene product with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring activated ligands, e.g., with altered activity, as well as naturally occurring and synthetic ligands, antagonists, agonists, small chemical molecules and the like. Such assays for inhibitors and activators include, e.g., applying putative modulator compounds to a cell expressing a receptor and then determining the functional effects on receptor signaling. Samples or assays comprising activated receptors that are treated with a potential activator, inhibitor, or modulator are compared to control samples without the inhibitor, activator, or modulator to examine the extent of inhibition. Control samples (untreated with inhibitors) can be assigned an activity value of 100%. Inhibition of activated samples is achieved when the activity value relative to the control is about 80%, optionally 50% or 25-0%. Activation of sample is achieved when the activity value relative to the control is 110%, optionally 150%, optionally 200-500%, or 1000-3000% higher.

“Pharmaceutically acceptable carrier (or medium)”, which can be used interchangeably with “biologically compatible carrier or medium”, refers to reagents, cells, compounds, materials, compositions, and/or dosage forms which are, within the scope of sound medical judgment, suitable for use in contact with the tissues of human beings and animals without excessive toxicity, irritation, allergic response, or other complication commensurate with a reasonable benefit/risk ratio. As described in greater detail herein, pharmaceutically acceptable carriers suitable for use in the present invention include liquids, semi-solid (e.g., gels) and solid materials (e.g., cell scaffolds). As used herein, the term biodegradable describes the ability of a material to be broken down (e.g., degraded, eroded, dissolved) in vivo. The term includes degradation in vivo with or without elimination (e.g., by resorption) from the body. The semi-solid and solid materials can be designed to resist degradation within the body (non-biodegradable) or they can be designed to degrade within the body (biodegradable, bioerodable). A biodegradable material can further be bioresorbable or bioabsorbable, i.e., it can be dissolved and absorbed into bodily fluids (water-soluble implants are one example), or degraded and ultimately eliminated from the body, either by conversion into other materials or breakdown and elimination through natural pathways.

This invention relies on routine techniques in the field of recombinant genetics. Basic texts disclosing the general methods of use in this invention include Sambrook et al., Molecular Cloning, A Laboratory Manual, 2^{nd} ed., 1989; Kriegler, Gene Transfer and Expression: A Laboratoiy Manual, 1990; and Ausubel et al., eds., Current Protocols in Molecular Biology, 1994; all of which are herein incorporated by reference for all purposes.

### 2. Overview

The processing requirements of two members of the TGF-β superfamily, CDMP1/GDF5 and Vg1, have been examined. Though injection of mRNAs encoding these genes is without effect in Xenopus laevis patterning assays (Tannahill and Melton, Development 106: 775-785, 1989; Dionne et al., Mol. Cell. Biol. 21: 636-643, 2001), CDMP1/GDF5 has been implicated in limb patterning (Storm et al., Nature 368: 639-643, 1994; Thomas et al., Nat. Genet. 17: 58-64, 1996; Thomas et al., Nat. Gent. 12: 315-317, 1997) and Vg1 in the induction of mesoderm and the dorsal organizing center (Thomsen and Melton, Cell 74: 433-441, 1993; Kessler and Melton, Methods Cell Biol. 36: 2155-2164, 1995). CDMP1/GDF5 is expressed predominantly in developing limbs at joint interzones and is known to play an important role in joint formation. Its absence leads to brachypodism in mice and at least two forms of acromesomelic chondrodyplasia in humans (Storm et al., Nature 368: 639-643, 1994; Thomas et al., Nat. Genet. 17: 58-64, 1996; Thomas et al., Nat. Gent. 12: 315-317, 1997). Moreover, unlike many other BMPs, CDMP1 is processed poorly by transfected COS cells (Thomas et al., Nat. Genet. 17: 58-64, 1997; Everman et al., Amer. J. Med. Genet. 112: 291-296, 2002).

The Nieuwkoop center is a region in the dorsal vegetal endoderm that is thought to release mesoderm-inducing activity that in turn establishes the dorsal organizing center. Vg1 has been considered a likely candidate for this signal (Spemann and Mangold, Roux's Arch Entwmech 100: 599-638, 1924; Weeks and Melton, Cell 51: 861-867, 1987, Thomsen and Melton, Cell 74: 433-441, 1993; Kessler and Melton, Methods Cell Biol. 36: 2155-2164, 1995; for review see Harland and Gerhart, Ann. Rev. Cell. Dev. Biol. 13: 611-667, 1997; De Robertis et al., Nat. Rev. Genet. 1: 171-181, 2000). However, attempts to identify significant amounts of native, mature active Vg1 protein in the embryo have been unsuccessful (Tannahill and Melton, Development 106: 775-785, 1989). As mentioned above, similar mechanisms are often used to control axial and limb patterning, prompting us to examine potential similarities between the CDMP1/GDF5 and Vg1 pathways. In addition, proteolytic processing of CDMP1/GDF5 and Vg1 was evaluated as described herein.

### 3. General Techniques

The nucleic acids used to practice this invention, whether RNA, iRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect, or plant cell expression systems.

Alternatively, these nucleic acids can be synthesized in vitro by well-known chemical synthesis techniques, as described in, e.g., Adams, J. Am. Chem. Soc. 105: 661, 1983; Belousov, Nucleic Acids Res. 25: 3440-3444, 1997; Frenkel, Free Radic. Biol. Med. 19: 373-380, 1995; Blommers, Biochemistry 33: 7886-7896, 1994; Narang, Meth. Enzymol. 68: 90, 1979; Brown Meth. Enzymol. 68: 109, 1979; Beaucage, Tetra. Lett. 22: 1859, 1981; U.S. Pat. No. 4,458,066.

The invention provides oligonucleotides comprising sequences of the invention, e.g., subsequences of the exemplary sequences of the invention. Oligonucleotides can include, e.g., single stranded poly-deoxynucleotides or two complementary polydeoxynucleotide strands which can be chemically synthesized.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2^{ND} ED.), Vols. 1-3, Cold Spring Harbor Laboratory, 1989; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y., 1993.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g., fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld, Nat. Genet. 15: 333-335, 1997; yeast artificial chromosomes (YAC); bacterial artificial chromosomes (BAC); P1 artificial chromosomes, see, e.g., Woon, Genomics 50: 306-316, 1998; P1-derived vectors (PACs), see, e.g., Kern, Biotechniques 23: 120-124, 1997; cosmids, recombinant viruses, phages, or plasmids.

The invention provides fusion proteins and nucleic acids encoding them. A morphogenic polypeptide of the invention can be fused to a heterologous peptide or polypeptide, such as N-terminal identification peptides that impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, the domain utilized in the FLAGS extension/affinity purification system (Immunex Corp., Seattle, Wash.), and cleavable linker sequences such as Factor Xa or enterokinase recognition sites (Invitrogen, San Diego, Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site. (See e.g., Williams, Biochemistry 34: 1787-1797, 1995; Dobeli, Protein Expr. Purif 12: 404-414, 1998). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll, DNA Cell. Biol. 12: 441-53, 1993.

### 4. Transcriptional Control Elements

The nucleic acids of the invention can be operatively linked to a promoter. A promoter can be one motif or an array of nucleic acid control sequences, which direct transcription of a nucleic acid. A promoter can include necessary nucleic acid sequences near the start site of transcription, such as, in the case of a polymerase II type promoter, a TATA element. A promoter also optionally includes distal enhancer or repressor elements, which can be, located as much as several thousand base pairs from the start site of transcription. A “constitutive” promoter is a promoter that is active under most environmental and developmental conditions. An “inducible” promoter is a promoter that is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

### 5. Expression Vectors and Cloning Vehicles

The invention provides expression vectors and cloning vehicles comprising nucleic acids of the invention, e.g., sequences encoding the proteins of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, fowl pox virus, pseudorabies and derivatives of SV40), P1-based artificial chromosomes, yeast plasmids, yeast artificial chromosomes, and any other vectors specific for specific hosts of interest (such as Bacillus, Aspergillus and yeast). Vectors of the invention can include chromosomal, non-chromosomal, and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The vector is then used to transform an appropriate host cell. Suitable recombinant expression systems include, but are not limited to, bacterial, mammalian, baculovirus/insect, vaccinia, Semliki Forest virus (SFV), Alphaviruses (such as Sindbis or Venezuelan Equine Encephalitis (VEE)), maiiunmalian, yeast, and Xenopus expression systems well known in the art. Particularly preferred expression systems are mammalian cell lines, vaccinia, Sindbis, eucaryotic layered vector initiation systems (e.g., U.S. Pat. No. 6,015,686, U.S. Pat. No. 5,814,482, U.S. Pat. No. 6,015,694, U.S. Pat. No. 5,789,245, EP 1029068A2, WO 9918226A2/A3, EP 00907746A2, WO 9738087A2, all herein incorporated by reference in their entireties for all purposes), insect, and yeast systems. Other expression systems include autologous or allogeneic human cells. Other expression systems include chondrocyte progenitor cells.

The invention provides libraries of expression vectors encoding polypeptides and peptides of the invention. These nucleic acids can be introduced into a genome or into the cytoplasm or a nucleus of a cell and expressed by a variety of conventional techniques, well described in the scientific and patent literature. See, e.g., Roberts, Nature 328: 731, 1987; Schneider, Protein Expr. Purif. 6435: 10, 1995; Sambrook or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors, or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors can also be employed which exploit advantageous merits of each of the parent vector properties. (See e.g., Feng, Nature Biotechnology 15: 866-870, 1997). Such viral genomes can be modified by recombinant DNA techniques to include the nucleic acids of the invention; and can be further engineered to be replication deficient, conditionally replicating, or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral, and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher, J. Virol. 66: 2731-2739, 1992; Johann, J. Virol. 66: 1635-1640, 1992). Adeno-associated virus (AAV)-based vectors can be used to infect cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada, Gene Ther. 3: 957-964, 1996. See also the Cellular Transfection and Gene Therapy section below.

“Expression cassette” as used herein refers to a nucleotide sequence capable of effecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression can also be used, e.g., enhancers.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination. Thus, expression cassettes also include plasmids, expression vectors, recombinant viruses, any form of recombinant “naked DNA” vector, and the like.

“Vector” is intended to refer to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Like retroviruses, transposons and transposon vectors can also be used to integrate sequences that an act as insertional mutagens. Also like retroviruses, transposons integrate by enzymatically catalyzed non-homologous recombination in which transposase enzymes catalyze the genomic integration and transposition of transposon DNA (Cui et al., J Mol Biol. 318: 1221-35, 2002; Izsvak et al., J Biol Chem. 277: 34581-8, Epub Jun. 24, 2002; see also Devine and Boeke, Nucl. Acids Res. 22: 3765-2772, 1994). By “transposon” or “transposable element” is meant a linear strand of DNA capable of integrating into a second strand of DNA which may be linear or may be a circularized plasmid.

### 6. Host Cells and Transformed Cells

The invention also provides a transformed cell comprising a nucleic acid sequence of the invention, e.g., a sequence encoding a polypeptide of the invention, or a vector of the invention. The host cell can be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS, Bowes melanoma, or any mouse or human cell line. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants, or amplifying the genes of the invention. Following transformation of a suitable host strain and growth of the host strain to an appropriate cell density, the selected promoter can be induced by appropriate means (e.g., temperature shift or chemical induction) and the cells can be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, or other types of adsorption chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa, and BHK cell lines.

The constructs in host cells can be used in a conventional manner to produce the gene product encoded by the recombinant sequence. Depending upon the host employed in a recombinant production procedure, the polypeptides produced by host cells containing the vector can be glycosylated or can be non-glycosylated. Polypeptides of the invention can or can not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct can be linearized prior to conducting an in vitro transcription reaction. The transcribed mRNA is then incubated with an appropriate cell-free translation extract, such as a rabbit retictulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

### 7. Amplification of Nucleic Acids

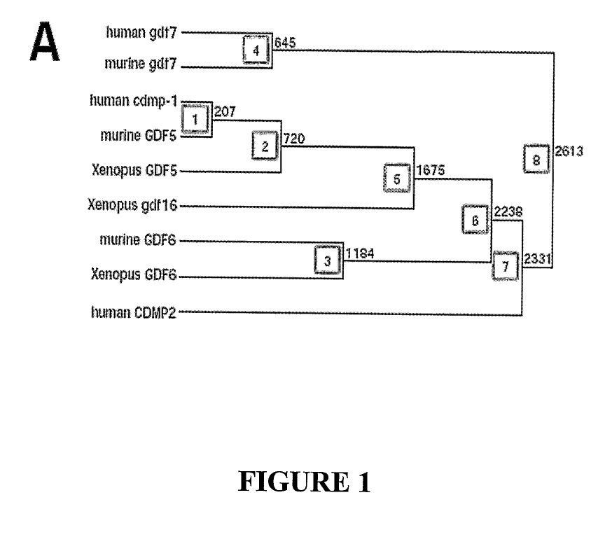

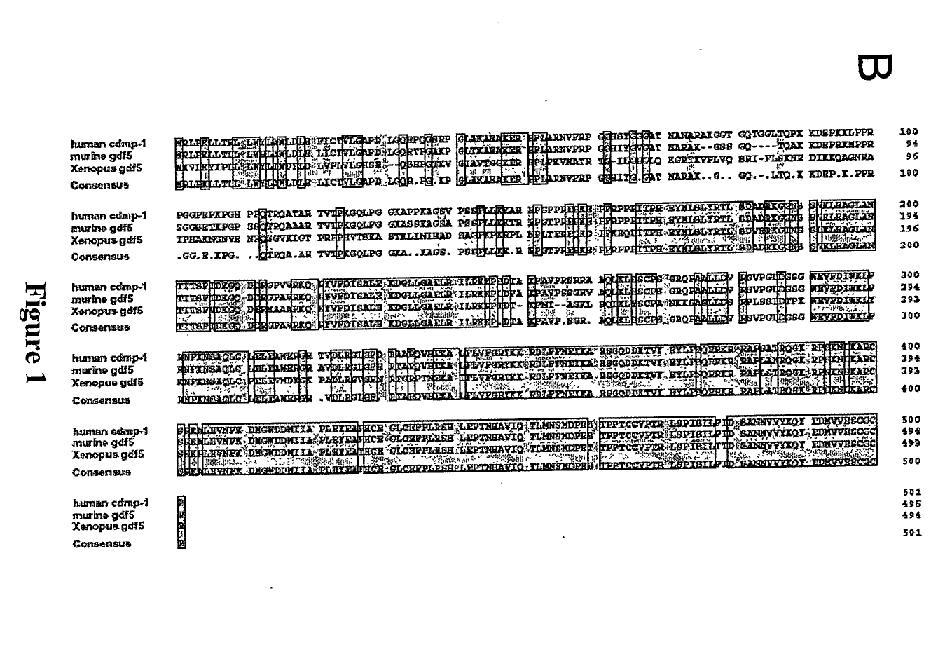

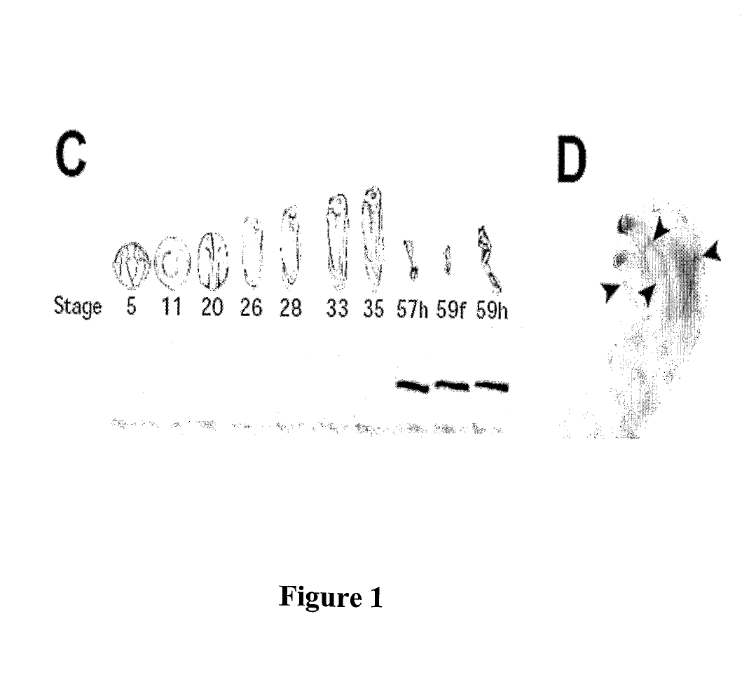

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides of the invention, e.g., primer pairs capable of amplifying nucleic acid sequences comprising the exemplary sequences in FIG. 1, or subsequences thereof.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4: 560, 1989; Landegren, Science 241: 1077, 1988; Barringer, Gene 89: 117, 1990); transcription amplification (see, e.g., Kwoh, Proc. Natl. Acad. Sci. USA 86: 1173, 1989); and, self-sustained sequence replication (see, e.g., Guatelli, Proc. Natl. Acad. Sci. USA 87: 1874, 1990); Q Beta replicase amplification (see, e.g., Smith, J. Clin. Microbiol. 35: 1477-1491, 1997), automated Q-beta replicase amplification assay (see, e.g., Burg, Mol. Cell. Probes 10: 257-271, 1996) and other RNA polymerase mediated techniques (e.g., NASBA, Cangene, Mississauga, Ontario); see also Berger, Methods Enzymol. 152: 307-316, 1987; Sambrook; Ausubel; U.S. Pat. Nos. 4,683,195 and 4,683,202; Sooknanan, Biotechnology 13: 563-564, 1995.

### 8. Hybridization of Nucleic Acids

The invention provides isolated or recombinant nucleic acids that hybridize under stringent conditions to an exemplary sequence of the invention, e.g., SEQ ID NO: 3 or 2, or the complement thereof, or a nucleic acid that encodes a polypeptide of the invention. In alternative aspects, the stringent conditions are highly stringent conditions, medium stringent conditions or low stringent conditions, as known in the art and as described herein. These methods can be used to isolate nucleic acids of the invention.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

“Selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one embodiment, a nucleic acid can be determined to be within the scope of the invention by its ability to hybridize under stringent conditions to a nucleic acid otherwise determined to be within the scope of the invention (such as the exemplary sequences described herein).

“Stringent hybridization conditions” refers to conditions under which a probe will hybridize to its target subsequence, typically in a complex mixture of nucleic acid, but not to other sequences in significant amounts (a positive signal (e.g., identification of a nucleic acid of the invention) is about 10 times background hybridization). Stringent conditions are sequence-dependent and will be different in different circumstances. Longer sequences hybridize specifically at higher temperatures. An extensive guide to the hybridization of nucleic acids is found in e.g., Sambrook, ed., Molecular Cloning: A Laboratory Manual (2^{nd} Ed.), Vols. 1-3, Cold Spring Harbor Laboratory, 1989; Current Protocols in Molecular Biology, Ausubel, ed. John Wiley & Sons, Inc., New York, 1997; Laboratory Techniques In Biochemistry And Molecular Biology Hybridization With Nucleic Acid Probes, Part I. Theory and Nucleic Acid Preparation, Tijssen, ed. Elsevier, N.Y., 1993.

Generally, stringent conditions are selected to be about 5-10° C. lower than the thermal melting point I for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide as described in Sambrook (cited below). For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. For selective or specific hybridization, a positive signal (e.g., identification of a nucleic acid of the invention) is about 10 times background hybridization. Stringent hybridization conditions that are used to identify nucleic acids within the scope of the invention include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. In the present invention, genomic DNA or cDNA comprising nucleic acids of the invention can be identified in standard Southern blots under stringent conditions using the nucleic acid sequences disclosed here. Additional stringent conditions for such hybridizations (to identify nucleic acids within the scope of the invention) are those which include a hybridization in a buffer of 40% formamide, 1 M NaCl, 1% SDS at 37° C.

However, the selection of a hybridization format is not critical—it is the stringency of the wash conditions that set forth the conditions that determine whether a nucleic acid is within the scope of the invention. Wash conditions used to identify nucleic acids within the scope of the invention include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen, and Ausubel for a description of SSC buffer and equivalent conditions.

### 9. Oligonucleotides Probes and Methods for Using Them

The invention also provides nucleic acid probes for identifying nucleic acids encoding a polypeptide that is a modulator of a morphogenic-signaling activity. In one aspect, the probe comprises at least 10 consecutive bases of a nucleic acid of the invention. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a sequence as set forth in a nucleic acid of the invention. The probes identify a nucleic acid by binding and/or hybridization. The probes can be used in arrays of the invention, see discussion below. The probes of the invention can also be used to isolate other nucleic acids or polypeptides.

### 10. Determining the Degree of Sequence Identity

The invention provides nucleic acids having at least 90%, 91%, 92%, 93%, 94%, 95%, 96%, 97%, 98%, 99% or more sequence identity to the sequences of the present invention as shown in FIG. 1. The invention provides polypeptides having at least 90%, 91%, 92%, 93%, 94%, 95%, 96%, 97%, 98%, 99% or more sequence identity sequences of the present invention as shown in FIG. 1. The sequence identities can be determined by analysis with a sequence comparison algorithm or by a visual inspection. Protein and/or nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2 or FASTA version 3.0t78 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Natl. Acad. Sci. U.S.A. 85: 2444, 1988, by computerized implementations of these algorithms (FASTDB (Intelligenetics), BLAST (National Center for Biotechnology Information; www.ncbi.nlm.nih.gov/ as given below), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., (1999 Suppl.), Current Protocols in Molecular Biology, Greene Publishing Associates and Wiley Interscience, N.Y., 1987)

A preferred example of an algorithm that is suitable for determining percent sequence identity and sequence similarity is the FASTA algorithm, which is described in Pearson & Lipman, Proc. Natl. Acad. Sci. U.S.A. 85: 2444, 1988. See also Pearson, Methods Enzymol. 266: 227-258, 1996. Preferred parameters used in a FASTA alignment of DNA sequences to calculate percent identity are optimized, BL50 Matrix 15: −5, k-tuple=2; joining penalty-40, optimization=28; gap penalty −12; gap length penalty=−2; and width=16.

Another preferred example of algorithm that is suitable for determining percent sequence identity and sequence similarity are the BLAST and BLAST 2.0 algorithms, which are described in Altschul et al., Nuc. Acids Res. 25: 3389-3402, 1977; and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89: 10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90: 5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35: 351-360, 1987. The method used is similar to the method described by Higgins & Sharp, CABIOS 5: 151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0. (Devereaux et al., Nuc. Acids Res. 12: 387-395, 1984).

Another preferred example of an algorithm that is suitable for multiple DNA and amino acid sequence alignments is the CLUSTALW program (Thompson et al., Nucl. Acids. Res. 22: 4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix. (Henikoff and Henikoff, Proc. Natl. Acad. Sci. U.S.A. 89:10915-10919, 1992).

“Sequence identity” refers to a measure of similarity between amino acid or nucleotide sequences, and can be measured using methods known in the art, such as those described below:

“Identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

“Substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the following sequence comparison algorithms or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred embodiment, the sequences are substantially identical over the entire length of the coding regions.

“Homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (e.g., SEQ ID NO: 3) to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. “Homology” refers specifically to whether two sequences share common ancestry (see Doolittle, R. F. (1987) Of URFs and ORFs, University Science Books, Mill Valley), generally based on one or more statistical tests for the significance of the sequence similarity under evaluation.

A “comparison window”, as used herein, includes reference to a segment of any one of the numbers of contiguous residues. For example, in alternative aspects of the invention, contiguous residues ranging anywhere from 20 to the full length of an exemplary polypeptide or nucleic acid sequence of the invention, are compared to a reference sequence of the same number of contiguous positions after the two sequences are optimally aligned. If the reference sequence has the requisite sequence identity to an exemplary polypeptide or nucleic acid sequence of the invention, e.g., at least 90%, 91%, 92%, 93%, 94%, 95%, 96%, 97%, 98%, 99% or more sequence identity to the sequences of the invention sequence (e.g., SEQ ID NO: 4), that sequence is within the scope of the invention.

Motifs which can be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

### 11. Inhibiting Expression of Polypeptides and Transcripts

The invention further provides for nucleic acids complementary to (e.g., antisense sequences to) the nucleic acid sequences of the invention. Antisense sequences are capable of inhibiting the transport, splicing or transcription of protein-encoding genes, e.g., the morphogenic nucleic acids encoding the polypeptides of the invention. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides that are able to either bind gene or message, in either case preventing or inhibiting the production or function of the protein. The association can be through sequence specific hybridization. Another useful class of inhibitors includes oligonucleotides that cause inactivation or cleavage of protein message. The oligonucleotide can have enzyme activity that causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

General methods of using antisense, ribozyme technology, and RNAi technology to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention. The term “RNAi” stands for RNA interference. This term is understood in the art to encompass technology using RNA molecules that can silence genes. (See, for example, McManus, et al., Nature Reviews Genetics 3: 737, 2002). In this application, the term “RNAi” encompasses molecules such as short interfering RNA (siRNA), microRNAs (mRNA), small temporal RNA (stRNA). Generally speaking, RNA interference results from the interaction of double-stranded RNA with genes.

### 12. Antisense Oligonucleotides

The invention provides antisense oligonucleotides capable of binding the message encoding the morphogenic polypeptide, which can inhibit polypeptide synthesis by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata, Toxicol Appl Pharmacol 144: 189-197, 1997; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

The invention provides a method of inhibiting expression of a gene encoding a morphogenic protein comprising the step of (i) providing a biological system in which expression of a gene encoding a morphogenic protein is to be inhibited; and (ii) contacting the system with an antisense molecule that hybridizes to a transcript encoding the morphogenic protein. In other embodiments, morphogenic proteins are inhibited. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression can be inducible and/or tissue or cell type-specific. The antisense molecule can be an oligonucleotide or a longer nucleic acid molecule. The invention provides such antisense molecules.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention. (See, e.g., Gold, J. Biol. Chem. 270: 13581-13584, 1995).

13. siRNA

RNA interference (RNAi) is a mechanism of post-transcriptional gene silencing mediated by double-stranded RNA (dsRNA), which is distinct from antisense and ribozyme-based approaches (see Jain, Pharmacogenomics 5: 239-42, 2004 for a review of RNAi and siRNA). RNA interference is useful in a method for treating a musculoskeletal disorder in a mammal by administering to the mammal a nucleic acid molecule (e.g., dsRNA) that hybridizes under stringent conditions to a morphogenic sequence as described herein, and attenuates expression of said target gene. dsRNA molecules are believed to direct sequence-specific degradation of mRNA in cells of various types after first undergoing processing by an RNase III-like enzyme called DICER (Bernstein et al., Nature 409: 363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

In general, a short, interfering RNA (siRNA) comprises an RNA duplex that is preferably approximately 19 basepairs long and optionally further comprises one or two single-stranded overhangs or loops. An siRNA can comprise two RNA strands hybridized together, or can alternatively comprise a single RNA strand that includes a self-hybridizing portion. siRNAs can include one or more free strand ends, which can include phosphate and/or hydroxyl groups. siRNAs typically include a portion that hybridizes under stringent conditions with a target transcript. One strand of the siRNA (or, the self-hybridizing portion of the siRNA) is typically precisely complementary with a region of the target transcript, meaning that the siRNA hybridizes to the target transcript without a single mismatch. In certain embodiments of the invention in which perfect complementarity is not achieved, it is generally preferred that any mismatches be located at or near the siRNA termini.

siRNAs have been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, Nat. Biotechnol. 20: 446-448, 2002; See also Yu et al., Proc. Natl. Acad. Sci., 99: 6047-6052, 2002; Sui et al, Proc. Natl. Acad. Sci. USA., 99: 5515-5520, 2002; Paddison et al., Genes and Dev. 16: 948-958, 2002; Brummelkamp et al., Science 296: 550-553, 2002; Miyagashi and Taira, Nat. Biotech. 20: 497-500, 2002; Paul et al., Nat. Biotech. 20: 505-508, 2002. As described in these and other references, the siRNA can consist of two individual nucleic acid strands or of a single strand with a self-complementary region capable of forming a hairpin (stem-loop) structure. A number of variations in structure, length, number of mismatches, size of loop, identity of nucleotides in overhangs, and the like, are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is preferred to target exons rather than introns, and it can also be preferable to select sequences complementary to regions within the 3′ portion of the target transcript. Generally it is preferred to select sequences that contain approximately equimolar ratio of the different nucleotides and to avoid stretches in which a single residue is repeated multiple times.

siRNAs can thus comprise RNA molecules having a double-stranded region approximately 19 nucleotides in length with 1-2 nucleotide 3′ overhangs on each strand, resulting in a total length of between approximately 21 and 23 nucleotides. As used herein, siRNAs also include various RNA structures that can be processed in vivo to generate such molecules. Such structures include RNA strands containing two complementary elements that hybridize to one another to form a stem, a loop, and optionally an overhang, preferably a 3′ overhang. Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and can be up to approximately 29 nucleotides in length. Loops of 4 nucleotides or greater are less likely subject to steric constraints than are shorter loops and therefore can be preferred. The overhang can include a 5′ phosphate and a 3′ hydroxyl. The overhang can but need not comprise a plurality of U residues, e.g., between 1 and 5 U residues. Classical siRNAs as described above trigger degradation of mRNAs to which they are targeted, thereby also reducing the rate of protein synthesis. In addition to siRNAs that act via the classical pathway, certain siRNAs that bind to the 3′ UTR of a template transcript can inhibit expression of a protein encoded by the template transcript by a mechanism related to but distinct from classic RNA interference, e.g., by reducing translation of the transcript rather than decreasing its stability. Such RNAs are referred to as microRNAs (mRNAs) and are typically between approximately 20 and 26 nucleotides in length, e.g., 22 nt in length. It is believed that they are derived from larger precursors known as small temporal RNAs (stRNAs) or mRNA precursors, which are typically approximately 70 nt long with an approximately 4-15 nt loop (See Grishok et al., Cell 106: 23-24, 2001; Hutvagner et al., Science 293: 834-838, 2001; Ketting, et al., Genes Dev., 15: 2654-2659, 2001). Endogenous RNAs of this type have been identified in a number of organisms including mammals, suggesting that this mechanism of post-transcriptional gene silencing can be widespread (Lagos-Quintana et al., Science 294: 853-858, 2001; Pasquinelli, Trends in Genetics 18: 171-173, 2002, and references in the foregoing two articles). MicroRNAs have been shown to block translation of target transcripts containing target sites in mammalian cells (Zeng et al., Molecular Cell 9: 1-20, 2002).

siRNAs such as naturally occurring or artificial (i.e., designed by humans) mRNAs that bind within the 3′ UTR (or elsewhere in a target transcript) and inhibit translation can tolerate a larger number of mismatches in the siRNA/template duplex, and particularly can tolerate mismatches within the central region of the duplex. In fact, there is evidence that some mismatches can be desirable or required as naturally occurring stRNAs frequently exhibit such mismatches as do mRNAs that have been shown to inhibit translation in vitro. For example, when hybridized with the target transcript such siRNAs frequently include two stretches of perfect complementarity separated by a region of mismatch. A variety of structures is possible. For example, the mRNA can include multiple areas of nonidentity (mismatch). The areas of nonidentity (mismatch) need not be symmetrical in the sense that both the target and the mRNA include nonpaired nucleotide. Typically the stretches of perfect complementarity are at least 5 nucleotides in length, e.g., 6, 7, or more nucleotides in length, while the regions of mismatch can be, for example, 1, 2, 3, or 4 nucleotides in length.

Hairpin structures designed to mimic siRNAs and mRNA precursors are processed intracellularly into molecules capable of reducing or inhibiting expression of target transcripts (McManus et al., RNA 8: 842-850, 2002). These hairpin structures, which are based on classical siRNAs consisting of two RNA strands forming a 19 bp duplex structure are classified as class I or class II hairpins. Class I hairpins incorporate a loop at the 5′ or 3′ end of the antisense siRNA strand (i.e., the strand complementary to the target transcript whose inhibition is desired) but are otherwise identical to classical siRNAs. Class II hairpins resemble mRNA precursors in that they include a 19 nt duplex region and a loop at either the 3′ or 5′ end of the antisense strand of the duplex in addition to one or more nucleotide mismatches in the stem. These molecules are processed intracellularly into small RNA duplex structures capable of mediating silencing. They appear to exert their effects through degradation of the target mRNA rather than through translational repression as is thought to be the case for naturally occurring mRNAs and stRNAs.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an siRNA, and any structure that generates such an siRNA (i.e., serves as a precursor to the RNA) is useful in the practice of the present invention.

In the context of the present invention, siRNAs are useful both for therapeutic purposes, e.g., to modulate the expression of a morphogenic molecule or protein in a subject at risk of or suffering from musculoskeletal disorder. In another aspect, the therapeutic treatment of a musculoskeletal target with an antibody, antisense vector, or double stranded RNA vector is also contemplated.

The invention therefore provides a method of inhibiting expression of a gene encoding a morphogenic protein comprising the step of (i) providing a biological system in which expression of a gene encoding morphogenic protein is to be inhibited; and (ii) contacting the system with an siRNA targeted to a transcript encoding the morphogenic protein. In other embodiments, morphogenic proteins are inhibited. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

By “biological system” is meant any vessel, well, or container in which biomolecules (e.g., nucleic acids, polypeptides, polysaccharides, lipids, and the like) are placed; a cell or population of cells; a tissue; an organ; an organism, and the like. Typically the biological system is a cell or population of cells, but the method can also be performed in a vessel using purified or recombinant proteins.

The invention provides siRNA molecules targeted to a transcript encoding any morphogenic protein or morphogenic-related protein. In particular, the invention provides siRNA molecules selectively or specifically targeted to a transcript encoding a polymorphic variant of such a transcript, wherein existence of the polymorphic variant in a subject is indicative of susceptibility to or presence of a musculoskeletal disorder. The terms “selectively” or “specifically targeted to”, in this context, are intended to indicate that the siRNA causes greater reduction in expression of the variant than of other variants (i.e., variants whose existence in a subject is not indicative of susceptibility to or presence of a musculoskeletal disorder). The siRNA, or collections of siRNAs, can be provided in the form of kits with additional components as appropriate.

14. Short Hairpin RNA (shRNA)

RNA interference (RNAi), a mechanism of post-transcriptional gene silencing mediated by double-stranded RNA (dsRNA), is useful in a method for treating a musculoskeletal disorder in a mammal by administering to the mammal a nucleic acid molecule (e.g., dsRNA) that hybridizes under stringent conditions to a morphogenic gene, and attenuates expression of said target gene. See Jain, Pharmacogenomics 5: 239-42, 2004 for a review of RNAi and siRNA. A further method of RNA interference in the present invention is the use of short hairpin RNAs (shRNA). A plasmid containing a DNA sequence encoding for a particular desired siRNA sequence is delivered into a target cell via transfection or virally mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression. Inhibition of protein expression by transfected siRNAs is a transient phenomenon that does not occur for times periods longer than several days. In some cases, this can be preferable and desired. In cases where longer periods of protein inhibition are necessary, shRNA mediated inhibition is preferable.

### 15. Full and Partial Length Antisense RNA Transcripts

Antisense RNA transcripts have a base sequence complementary to part or all of any other RNA transcript in the same cell. Such transcripts have been shown to modulate gene expression through a variety of mechanisms including the modulation of RNA splicing, the modulation of RNA transport and the modulation of the translation of mRNA (Denhardt, Ann N Y Acad. Sci. 660: 70, 1992; Nellen, Trends Biochem. Sci. 18: 419, 1993; Baker and Monia, Biochem. Biophys. Acta, 1489: 3, 1999; Xu et al., Gene Therapy 7: 438, 2000; French and Gerdes, Curr. Opin. Microbiol. 3: 159, 2000; Terryn and Rouze, Trends Plant Sci. 5: 1360, 2000).

### 16. Antisense RNA and DNA Oligonucleotides

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but can range from 10 up to approximately 50 nucleotides in length. Binding typically reduces or inhibits the function of the target nucleic acid. For example, antisense oligonucleotides can block transcription when bound to genomic DNA, inhibit translation when bound to mRNA, and/or lead to degradation of the nucleic acid. Reduction in expression of a morphogenic or morphogenic polypeptide can be achieved by the administration of antisense nucleic acids or peptide nucleic acids comprising sequences complementary to those of the mRNA that encodes the polypeptide. Antisense technology and its applications are well known in the art and are described in Phillips, M. I. (ed.) Antisense Technology, Methods Enzymol., 2000, Volumes 313 and 314, Academic Press, San Diego, and references mentioned therein. See also Crooke, S. (ed.) “Antisense Drug Technology: Principles, Strategies, and Applications” (1^{st} Edition) Marcel Dekker; and references cited therein.

Antisense oligonucleotides can be synthesized with a base sequence that is complementary to a portion of any RNA transcript in the cell. Antisense oligonucleotides can modulate gene expression through a variety of mechanisms including the modulation of RNA splicing, the modulation of RNA transport and the modulation of the translation of mRNA (Denhardt, Ann N Y Acad. Sci. 660: 70, 1992). Various properties of antisense oligonucleotides including stability, toxicity, tissue distribution, and cellular uptake and binding affinity can be altered through chemical modifications including (i) replacement of the phosphodiester backbone (e.g., peptide nucleic acid, morpholino-oligonucleotides, phosphorothioate oligonucleotides, and phosphoramidate oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C) (Wagner, Nat. Medicine 1: 1116, 1995; Varga et al., Iimmun. Lett. 69: 217, 1999; Neilsen, Curr. Opin. Biotech. 10: 71, 1999; Woolf, Nucleic Acids Res. 18: 1763, 1990).

The invention provides a method of inhibiting expression of a gene encoding a musculoskeletal disorder comprising the step of (i) providing a biological system in which expression of a gene encoding a morphogenic protein is to be inhibited; and (ii) contacting the system with an antisense molecule that hybridizes to a transcript encoding the morphogenic molecule or morphogenic protein. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression can be inducible and/or tissue or cell type-specific. The antisense molecule can be an oligonucleotide or a longer nucleic acid molecule. The invention provides such antisense molecules.

### 17. Inhibitory Ribozymes

The invention provides ribozymes capable of binding message which can inhibit polypeptide activity by targeting mRNA, e.g., inhibition of polypeptides with morphogenic activity. Thus, RNA and DNA enzymes can be designed to cleave to any RNA molecule, thereby increasing its rate of degradation (Cotten and Birnstiel, EMBO J. 8: 3861-3866, 1989; Usman et al., Nucl. Acids Mol. Biol. 10: 243, 1996; Usman et al., Curr. Opin. Struct. Biol. 1: 527, 1996; Sun et al., Pharmacol. Rev. 52: 325, 2000).

Strategies for designing ribozymes and selecting the protein-specific antisense sequence for targeting are well described in the scientific and patent literature, and the skilled artisan can design such ribozymes using the novel reagents of the invention.

Ribozymes act by binding to a target RNA through the target RNA binding portion of a ribozyme that is held in close proximity to an enzymatic portion of the RNA that cleaves the target RNA. Thus, the ribozyme recognizes and binds a target RNA through complementary basepairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation, or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but can also be formed in the motif of a hairpin, hepatitis delta virus, group I intron or RnaseP-like RNA (in association with an RNA guide sequence). Examples of such hammerhead motifs are described by Rossi, Aids Research and Human Retroviruses 8: 183, 1992; hairpin motifs by Hampel, Biochemistry 28: 4929, 1989, and Hampel, Nuc. Acids Res. 18: 299, 1990; the hepatitis delta virus motif by Perrotta, Biochemistry 31: 16, 1992; the RnaseP motif by Guerrier-Takada, Cell 35: 849, 1983; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

The invention provides a method of inhibiting expression of a gene encoding a morphogenic gene (such as comprising the step of (i) providing a biological system in which expression of a gene encoding a morphogenic protein is to be inhibited; and (ii) contacting the system with a ribozyme that hybridizes to a transcript encoding the morphogenic molecule or morphogenic protein and directs cleavage of the transcript. According to certain embodiments of the invention the biological system comprises a cell, and the contacting step comprises expressing the ribozyme in the cell. According to certain embodiments of the invention the biological system comprises a subject, e.g., a mammalian subject such as a mouse or human, and the contacting step comprises administering the ribozyme to the subject or comprises expressing the ribozyme in the subject. The expression can be inducible and/or tissue or cell-type specific according to certain embodiments of the invention. The invention provides ribozymes designed to cleave transcripts encoding morphogenic molecules or morphogenic proteins, or polymorphic variants thereof, as described above.

### 18. Transgenic and “Knockout” Non-Human Animals

The invention provides transgenic non-human animals comprising a nucleic acid, a polypeptide, an expression cassette or vector or a transfected or transformed cell of the invention. The transgenic non-human animals can be, e.g., goats, rabbits, sheep, pigs, cows, rats and mice, comprising the nucleic acids of the invention. A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. Native expression in an animal can be reduced by providing an amount of antisense RNA or DNA effective to reduce expression of the receptor.

These animals can be used, e.g., as in vivo models to study modulators of a morphogenic-signaling activity, or, as models to screen for agents that change the morphogenic—signaling activity in vivo.

In one aspect, the inserted transgenic sequence is a sequence of the invention designed such that it does not express a functional morphogenic polypeptide. The defect can be designed to be on the transcriptional, translational, and/or the protein level.

The coding sequences for the polypeptides, the morphogenic polypeptides, or mutant polypeptide to be expressed in the transgenic non-human animals can be designed to be constitutive, or, under the control of tissue-specific, developmental-specific, or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs, and cows. See also, e.g., Pollock, J. Immunol. Methods 231: 147-157, 1999, describing the production of recombinant proteins in the milk of transgenic dairy animals; Baguisi, Nat. Biotechnol. 17: 456-461, 1999, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP). One exemplary method to produce genetically altered non-human animals is to genetically modify embryonic stem cells. The modified cells are injected into the blastocell of a blastocyst. This is then grown in the uterus of a pseudopregnant female. In order to readily detect chimeric progeny, the blastocysts can be obtained from a different parental line than the embryonic stem cells. For example, the blastocysts and embryonic stem cells can be derived from parental lines with different hair color or other readily observable phenotype. The resulting chimeric animals can be bred in order to obtain non-chimeric animals that have received the modified genes through germ-line transmission. Techniques for the introduction of embryonic stem cells into blastocysts and the resulting generation of transgenic animals are well known.

Because cells contain more than one copy of a gene, the cell lines obtained from a first round of targeting are likely to be heterozygous for the targeted allele. Homozygosity, in which both alleles are modified, can be achieved in a number of ways. In one approach, a number of cells in which one copy has been modified are grown. They are then subjected to another round of targeting using a different selectable marker. Alternatively, homozygotes can be obtained by breeding animals heterozygous for the modified allele, according to traditional Mendelian genetics. In some situations, it can be desirable to have two different modified alleles. This can be achieved by successive rounds of gene targeting or by breeding heterozygotes, each of which carries one of the desired modified alleles. See, e.g., U.S. Pat. No. 5,789,215.

Various methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82: 4438-4442, 1985). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), Harlan Sprague Dawley (Indianapolis, Ind.), and the like.

The procedures for manipulation of the rodent embryo and for microinjection of DNA into the pronucleus of the zygote are well known to those of ordinary skill in the art (Hogan et al., supra). Microinjection procedures for fish, amphibian eggs, and birds are detailed in Houdebine and Chourrout, Experientia 47: 897-905, 1991. Other procedures for introduction of DNA into tissues of animals are described in U.S. Pat. No. 4,945,050 (Sanford et al., Jul. 30, 1990).

By way of example only, to prepare a transgenic mouse, female mice are induced to superovulate. Females are placed with males, and the mated females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice (Hammer et al., Cell 63: 1099-1112, 1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (Teratocarcinomas and Embryonic Stem Cells, A Practical Approach, E. J. Robertson, ed., IRL Press, 1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombination (Capecchi, Science 244: 1288-1292, 1989). Methods for positive selection of the recombination event (i.e., neo resistance) and dual positive-negative selection (i.e., neo resistance and gancyclovir resistance) and the subsequent identification of the desired clones by PCR have been described by Capecchi, supra and Joyner et al., Nature 338: 153-156, 1989, the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (Houdebine and Chourrout, supra; Pursel et al., Science 244: 1281-1288, 1989; and Simms et al., Bio/Technology 6: 179-183, 1988).

### 19. Functional Knockouts

The invention provides non-human animals that do not express their endogenous morphogenic polypeptides, or, express their endogenous morphogenic polypeptides at lower than wild type levels (thus, while not completely “knocked out” their morphogenic activity is functionally “knocked out”). The invention also provides “knockout animals” and methods for making and using them. For example, in one aspect, the transgenic or modified animals of the invention comprise a “knockout animal,” e.g., a “knockout mouse,” engineered not to express an endogenous gene, e.g., an endogenous morphogenic gene, which is replaced with a gene expressing a polypeptide of the invention, or, a fusion protein comprising a polypeptide of the invention. Thus, in one aspect, the inserted transgenic sequence is a sequence of the invention designed such that it does not express a functional morphogenic polypeptide. The defect can be designed to be on the transcriptional, translational and/or the protein level. Because the endogenous morphogenic gene has been “knocked out,” only the inserted polypeptide of the invention is expressed.

A “knock-out animal” is a specific type of transgenic animal having cells that contain DNA containing an alteration in the nucleic acid sequence that reduces the biological activity of the polypeptide normally encoded therefrom by at least 80% compared to the unaltered gene. The alteration can be an insertion, deletion, frameshift mutation, missense mutation, introduction of stop codons, mutation of critical amino acid residue, removal of an intron junction, and the like. Preferably, the alteration is an insertion or deletion, or is a frameshift mutation that creates a stop codon. Typically, the disruption of specific endogenous genes can be accomplished by deleting some portion of the gene or replacing it with other sequences to generate a null allele. Cross-breeding mammals having the null allele generates a homozygous mammals lacking an active copy of the gene.

A number of such mammals have been developed, and are extremely helpful in medical development. For example, U.S. Pat. No. 5,616,491 describes knock-out mice having suppression of CD28 and CD45. Procedures for preparation and manipulation of cells and embryos are similar to those described above with respect to transgenic animals, and are well known to those of ordinary skill in the art.

A knock out construct refers to a uniquely configured fragment of nucleic acid that is introduced into a stem cell line and allowed to recombine with the genome at the chromosomal locus of the gene of interest to be mutated. Thus, a given knock out construct is specific for a given gene to be targeted for disruption. Nonetheless, many common elements exist among these constructs and these elements are well known in the art. A typical knock out construct contains nucleic acid fragments of about 0.5 kb to about 10.0 kb from both the 5′ and the 3′ ends of the genomic locus encoding the gene to be mutated. An intervening fragment of nucleic acid encoding a positive selectable marker, such as the neomycin resistance gene, typically separates these two fragments. The resulting nucleic acid fragment, consisting of a nucleic acid from the extreme 5′ end of the genomic locus linked to a nucleic acid encoding a positive selectable marker that is in turn linked to a nucleic acid from the extreme 3′ end of the genomic locus of interest, omits most of the coding sequence for the gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug.

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgenic animal expressing a polypeptide of the targeted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of the polypeptide product of the wild-type gene from which it was derived.

Each knockout construct to be inserted into the cell must first be in the linear form. Therefore, if the knockout construct has been inserted into a vector, linearization is accomplished by digesting the DNA with a suitable restriction endonuclease selected to cut only within the vector sequence and not within the knockout construct sequence. For insertion, the knockout construct is added to the ES cells under appropriate conditions for the insertion method chosen, as is known to the skilled artisan. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion can be accomplished in a variety of ways known to the skilled artisan, however a preferred method is by microinjection. For microinjection, about 10-30 cells are collected into a micropipette and injected into embryos that are at the proper stage of development to permit integration of the foreign ES cell containing the knockout construct into the developing embryo. For instance, the transformed ES cells can be microinjected into blastocysts. The suitable stage of development for the embryo used for insertion of ES cells is very species dependent, however for mice it is about 3.5 days post conception (dpc). The embryos are obtained by perfusing the uterus of pregnant females. Suitable methods for accomplishing this are known to the skilled artisan. After the ES cell has been introduced into the embryo, the embryo can be implanted into the uterus of a pseudopregnant foster mother for gestation as described above.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a target gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

The functional morphogenic “knockout” non-human animals of the invention are of several types. Some non-human animals of the invention that are functional morphogenic “knockouts” express sufficient levels of a morphogenic inhibitory nucleic acid, e.g., antisense sequences or ribozymes of the invention, to decrease the levels or knockout the expression of functional polypeptide. Some non-human animals of the invention that are functional morphogenic “knockouts” express sufficient levels of a morphogenic dominant negative polypeptide such that the effective amount of free endogenous active morphogenic is decreased. Some non-human animals of the invention that are functional morphogenic “knockouts” express sufficient levels of an antibody of the invention, e.g., a morphogenic antibody, such that the effective amount of free endogenous active morphogenic protein is decreased. Some non-human animals of the invention that are functional morphogenic “knockouts” are “conventional” knockouts in that their endogenous morphogenic gene has been disrupted or mutated.

Functional morphogenic “knockout” non-human animals of the invention also include the inbred mouse strain of the invention and the cells and cell lines derived from these mice.

The invention provides methods for treating a subject with a musculoskeletal disorder. The method comprises providing an inhibitor of a morphogenic activity, e.g., a nucleic acid (e.g., antisense, ribozyme) or a polypeptide (e.g., antibody or dominant negative) of the invention. The inhibitor is administered in sufficient amounts to the subject to inhibit the expression of morphogenic polypeptides.

### 20. Inbred Mouse Strains

The invention provides an inbred mouse and an inbred mouse strain that can be generated as described herein and bred by standard techniques, see, e.g., U.S. Pat. Nos. 6,040,495; 5,552,287.

In order to screen for mutations with recessive effects a number of strategies can be used, all involving a further two generations. For example, male G1 mice can be bred to wild-type female mice. The resulting progeny (G2 mice) can be interbred or bred back to the G1 father. The G3 mice that result from these crosses will be homozygotes for mutations in a small number of genes (3-6) in the genome, but the identity of these genes is unknown. With enough G3 mice, a good sampling of the genome should be present.

### 21. Animal Models for Joint Repair

Various animal models have been used for evaluation of possible clinical approaches to joint repair. The most widely used of these include the goat, sheep, and horse, each of which has certain capabilities and limitations (for review, see Reinholz et al., Biomaterials 25: 1511-1521, 2004, this reference is herein incorporated by reference for all purposes; note also detailed information on this subject in connection with the March 3-4 Meeting of the FDA Cellular, Tissue, and Gene Therapies Advisory committee at http://www.fda.gov/ohrms/dockets/ac/cber05.html#CellularTissueGeneTherapies/)

### 22. Peptides and Polypeptides

The invention provides isolated or recombinant polypeptides comprising an amino acid sequence having at least 95%, 96%, 97%, 98%, 99% or more sequence identity to a sequence of SEQ ID NO: 3 or 2, over a region of at least about 100, 150, 200, 250, 300, 350, 400, 450, 500, 550, 600, 650, 700, 750, 800, 850, 900, 950, 1000, 1100 or more residues, or, the full length of the polypeptide, or, a polypeptide encoded by a nucleic acid of the invention. In one aspect, the polypeptide comprises SEQ ID NO: 4. The invention provides methods for inhibiting the activity of morphogenic polypeptides, e.g., a polypeptide of the invention. The invention also provides methods for screening for compositions that inhibit the activity of, or bind to (e.g., bind to the active site), of morphogenic polypeptides, e.g., a polypeptide of the invention.

In one aspect, the invention provides morphogenic polypeptides (and the nucleic acids encoding them) where one, some or all of the morphogenic polypeptides replacement with substituted amino acids. In one aspect, the invention provides methods to disrupt the interaction of morphogenic polypeptides with other proteins, in antigen presentation pathways.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for modulators of a morphogenic activity and for agents that can ameliorate a musculoskeletal disorder. Polypeptides (e.g., antibody or dominant negative) of the invention can also be used to tolerize a subject to an antigen for, e.g., inducing humoral or cellular anergy to an immunogen.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, it has a morphogenic-signaling activity. A mimetic composition can also be within the scope of the invention if it can inhibit an activity of a morphogenic polypeptides of the invention, e.g., be a dominant negative mutant or, bind to an antibody of the invention.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola, Chemistry and Biochemistry of Amino Acids, Peptides and Proteins 7: 267-357, 1983).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (□adioimmu)-acetic acid, or (□adioimmu)alkyl-acetic acid, where alkyl is defined above. Nitrile derivative (e.g., containing the CN-moiety in place of COOH) can be substituted for □adioimmuno or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be oxidated to form, e.g., methionine sulfoxide. Mimetics of □adioim include, e.g., pipecolic acid, thiazolidine carboxylic acid, 3- or 4-hydroxy □adioim, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of □adioim and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for radioimmunoassay). One or more amino acids can be deleted, for example, from a morphogenic polypeptide of the invention, resulting in modification of the structure of the polypeptide, without significantly altering its biological activity. For example, amino- or carboxyl-terminal, or internal, amino acids that are not required for a morphogenic-signaling activity can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.) and the inclusion of cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (See e.g., Williams, Biochemistry 34: 1787-1797, 1995; Dobeli, Protein Expr. Purif 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll, DNA Cell. Biol., 12: 441-53, 1993.

The terms “polypeptide” and “protein” as used herein, refer to amino acids joined to each other by peptide bonds or modified peptide bonds, i.e., peptide isosteres, and can contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

As used herein, the term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids that have been purified from genomic DNA or from other sequences in a library or other environment by at least one, two, three, four, five, or more orders of magnitude.

### 23. Fusion Proteins

Antibodies to morphogenic gene products (e.g., a morphogenic protein) can be used to generate fusion proteins. For example, the antibodies of the present invention, when fused to a second protein, can be used as an antigenic tag. Antibodies raised against a morphogenic gene product (e.g., a morphogenic protein) can be used to indirectly detect the second protein by binding to the polypeptide.

Examples of domains that can be fused to polypeptides include not only heterologous signal sequences, but also other heterologous functional regions. The fusion does not necessarily need to be direct, but can occur through linker sequences.

Moreover, fusion proteins can also be engineered to improve characteristics of the polypeptide. For instance, a region of additional amino acids, particularly charged amino acids, can be added to the N-terminus of the polypeptide to improve stability and persistence during purification from the host cell or subsequent handling and storage. Other fusions might be constructed to direct the polypeptide to particular subcellular compartments. Also, peptide moieties can be added to the polypeptide to facilitate purification. Such regions can be removed prior to final preparation of the polypeptide. The addition of peptide moieties to facilitate handling of polypeptides are familiar and routine techniques in the art.

Moreover, antibody compositions to a morphogenic proteins, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. EP A 394,827; Traunecker et al., Nature, 331: 84-86, 1988. Fusion proteins having disulfide-linked dimeric structures (due to the IgG) can also be more efficient in binding and neutralizing other molecules, than the monomeric secreted protein or protein fragment alone. Fountoulakis et al., J. Biochem. 270: 3958-3964, 1995.

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion can hinder therapy and diagnosis if the fusion protein is used as an antigen for immunizations. In drug discovery, for example, human proteins, such as hIL-5, have been fused with Fc portions for the purpose of high throughput screening assays to identify antagonists of hIL-5. Bennett et al., J. Molecular Recognition 8: 52-58, 1995; Johanson et al., J. Biol. Chem., 270: 9459-9471, 1995.

Moreover, the polypeptides can be fused to marker sequences, such as a peptide that facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824, 1989, for instance, hexa-histidine provides for convenient purification of the fusion protein. Another peptide tag useful for purification, the “HA” tag, corresponds to an epitope derived from the influenza hemagglutinin protein (Wilson et al., Cell 37: 767, 1984).

Additional fusion proteins of the invention can be generated through the techniques of gene-shuffling, motif-shuffling, exon-shuffling, or codon-shuffling (collectively referred to as “DNA shuffling”). DNA shuffling can be employed to modulate the activities of polypeptides of the present invention thereby effectively generating agonists and antagonists of the polypeptides. See, for example, U.S. Pat. Nos. 5,605,793; 5,811,238; 5,834,252; 5,837,458; Patten, et al., Curr. Opinion Biotechnol., 8: 724-733, 1997; Harayama, Trends Biotechnol., 16: 76-82, 1998; Hansson, et al., J. Mol. Biol., 287: 265-276, 1999; Lorenzo, et al., Biotechniques, 24: 308-313, 1998. (Each of these documents is hereby incorporated by reference). In one embodiment, one or more components, motifs, sections, parts, domains, fragments, and the like, of coding polynucleotides of the invention, or the polypeptides encoded thereby can be recombined with one or more components, motifs, sections, parts, domains, fragments, and the like of one or more heterologous molecules.

Thus, any of these above fusions can be engineered using the polynucleotides or the polypeptides of the present invention.

### 24. Therapeutic Applications

The compounds and modulators identified by the methods of the present invention can be used in a variety of methods of treatment. Thus, the present invention provides compositions and methods for treating a musculoskeletal disorder including all disorders related to bone, muscle, ligaments, tendons, cartilage, and joints. Treatment of a musculoskeletal disease or disorder is within the ambit of regenerative medicine. For example, disorders requiring spinal fixation, spinal stabilization, repair of segmental defects in the body (such as in long bones and flat bones), disorders of the vertebrae and discs including, but not limited to, disruption of the disc annulus such as annular fissures, chronic inflammation of the disc, localized disc herniations with contained or escaped extrusions, and relative instability of the vertebrae surrounding the disc are musculoskeletal disorders. Musculoskeletal disorders also include sprains, strains and tears of ligaments, tendons, muscles, and cartilage; tendonitis, tenosynovitis, fibromyalgia, osteoarthritis, rheumatoid arthritis, polymyalgia rheumatica, bursitis, acute and chronic back pain and osteoporosis, sports injuries and work related injuries including sprains, strains and tears of ligaments, tendons, muscles, and cartilage; carpal tunnel syndrome, DeQuervains's disease, trigger finger, tennis elbow, rotator cuff injuries, and ganglion cysts. In addition, musculoskeletal disorders include genetic diseases of the musculoskeletal system such as osteogenesis imperfecta, Duchenne, and other muscular dystrophies. Pain is the most common symptom and is frequently caused by injury or inflammation. Besides pain, other symptoms such as stiffness, tenderness, weakness, and swelling or deformity of affected parts are manifestations of musculoskeletal disorders.

Preferably, treatment using a polypeptide or polynucleotide of the present invention could either be by administering an effective amount of a polypeptide to the patient, or by removing cells from the patient, supplying the cells with a polynucleotide or polynucleotides of the present invention, and returning the engineered cells to the patient (ex vivo therapy).

### 25. Cellular Transfection and Gene Therapy

Another aspect of the present invention is to use gene therapy methods for treating or preventing disorders, diseases, and conditions. The gene therapy methods relate to the introduction of nucleic acid (DNA, RNA and antisense DNA or RNA) sequences into an animal to achieve expression of the polypeptide or polypeptides of the present invention. This method requires one or more polynucleotides encoding a polypeptide(s) of the present invention operatively linked to a promoter and any other genetic elements necessary for the expression of the polypeptide by the target tissue.

In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik, et al., Proc. Natl. Acad. Sci. U.S.A., 83: 4143-4146, 1986). The oligonucleotides can be modified to enhance their uptake, e.g., by substituting their negatively charged phosphodiester groups by uncharged groups.

The present invention provides the nucleic acids of GDF5/CDMP-1, SPC1 and SPC6 for the transfection, transduction, or other genetic modification of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid for GDF5/CDMP-1, SPC1 and SPC6, under the control of a promoter(s), then expresses a GDF5/CDMP-1, SPC1 and SPC6 of the present invention. The compositions are administered to a patient in an amount sufficient to elicit a therapeutic response in the patient. An amount adequate to accomplish this is defined as “therapeutically effective dose or amount.” Such gene therapy procedures have been used to correct acquired and inherited genetic defects, cancer, and viral infection in a number of contexts. The ability to express artificial genes in humans may facilitate the prevention and/or cure of important human diseases, including diseases which are not amenable to treatment by other therapies (for a review of gene therapy procedures, see Anderson, Science 256: 808-813, 1992; Nabel & Felgner, TIBTECH 11: 211-217, 1993; Mitani & Caskey, TIBTECH 11: 162-166, 1993; Mulligan, Science 260: 926-932, 1993; Dillon, TIBTECH 11: 167-175, 1993; Miller, Nature 357: 455-460, 1992; Van Brunt, Biotechnology 6(10): 1149-1154, 1998; Vigne, Restorative Neurology and Neuroscience 8: 35-36, 1995; Kremer & Perricaudet, British Medical Bulletin 51: 31-44, 1995; Haddada et al., In Current Topics in Microbiology and Immunology (Doerfier & Bohm eds., 1995); and Yu, Gene Therapy 1: 13-26, 1994). For a review of orthopaedic gene therapy, see Evans, Clin Orthop Relate Res 429: 316-29, 2004 and Evans, J. Rheumatol Suppl 72: 17-20, 2005; general reference for mechanisms of retroviral infection, replication, and integration: Coffin, In: Retroviruses. Cold Spring Harbor Laboratory Press, Plainview, N.Y., 1997; Varmus, Cell 25: 23-36, 1981; Friedrich, Methods Enzylnol. 225: 681-701, 1993; Gossler, Science 244: 463-5, 1989; Friedrich, Genes Dev. 5: 1513-23, 1991; von Melchner, Genes Dev. 6: 919-27, 1992; King, Science 228: 554-8, 1985; Hubbard, J Biol. Chem. 269: 3717-24, 1994; these references are herein incorporated by reference for all purposes.

The invention provides a number of methods for modulating musculoskeletal disorders in a subject. As disclosed herein, the invention provides a method of modulating musculoskeletal disorders in a subject, the method comprising the step of administering to the subject a therapeutically effective amount of a nucleic acid encoding a hCDMP-1 polypeptide variant, wherein the nucleic acid hybridizes under stringent conditions to a nucleic acid encoding a polypeptide having an amino acid sequence of SEQ ID NO: 4.

Other methods are provided for modulating musculoskeletal disorders in a subject comprising the steps of: (a) isolating cells to be implanted into said subject (b) introducing into the cells the recombinant expression system of claim 26; and (c) implanting the cells containing the recombinant expression system into said subject.

Additional methods for modulating musculoskeletal disorders in a subject in need thereof are provided, comprising: (a) selecting the patient in need thereof; (b) isolating cells from the patient, wherein the cells express CDMP-1 and introducing into the cells a first nucleotide sequence encoding SPC1 and a second nucleotide sequence encoding SPC6, wherein the first and second nucleotide sequences are independently operatively linked to transcription controlling nucleotide sequences in the isolated cells; and (c) readministering the cells to the patient.

Nucleic acid constructs can be designed in accordance with the principles, materials, and methods disclosed in the patent documents and scientific literature cited herein, each of which is incorporated herein by reference, with modifications and further exemplification as described herein. Components of the constructs can be prepared in conventional ways, where the coding sequences and regulatory regions can be isolated, as appropriate, ligated, cloned in an appropriate cloning host, and analyzed by restriction or sequencing other convenient means. Particularly, using PCR, individual fragments including all or portions of a functional unit can be isolated, where one or more mutations can be introduced using “primer repair;” ligation, in vitro mutagenesis, and the like, as appropriate. In the case of DNA constructs encoding fusion proteins, DNA sequences encoding individual domains and sub-domains can be joined such that they constitute a single open reading frame encoding a fusion protein capable of being translated in cells or cell lysates into a single polypeptide harboring all component domains. The DNA construct encoding the fusion protein can then be placed into a vector that directs the expression of the protein in the appropriate cell type(s). Alternatively, the desired DNA constructs can be generated by homologous recombination in bacteria using commercially available techniques that are well described in the literature (Zhang, Nature Biotechnology 18: 1314-1317, 2000). Accordingly, fusion proteins of the present invention can be generated by homologous recombination into endogenous gene loci. For biochemical analysis of the encoded chimera, it can be desirable to construct plasmids that direct the expression of the protein in bacteria or in reticulocyte-lysate or other in vitro translation systems. For use in the production of proteins in mammalian cells, the protein-encoding sequence can be introduced into an expression vector that directs expression in these cells. Expression vectors suitable for such uses are well known in the art. Various sorts of such vectors are commercially available.

Methods of non-viral delivery of recombinant constructs of the invention include, for example, lipofection, microinjection, biolistics, virosomes, liposomes, immunoliposomes, polycation or lipid:nucleic acid conjugates, naked DNA, artificial virions, and agent-enhanced uptake of DNA. Lipofection is described in, for example, U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270: 404-410, 1995; Blaese, Cancer Gene Ther. 2: 291-297, 1995; Behr, Bioconjugate Chem. 5: 382-389, 1994; Remy, Bioconjugate Chem. 5: 647-654, 1994; Gao, Gene Therapy 2: 710-722, 1995; Ahmad, Cancer Res. 52: 4817-4820, 1992; U.S. Pat. Nos. 4,186,183, 4,217,344, 4,235,871, 4,261,975, 4,485,054, 4,501,728, 4,774,085, 4,837,028, and 4,946,787).

In certain embodiments, the polynucleotide constructs are complexed in a liposome preparation. Liposomal preparations for use in the present invention include cationic (positively charged), anionic (negatively charged), and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner, Proc. Natl. Acad. Sci. USA 84: 7413-7416, 1987, which is herein incorporated by reference); mRNA (Malone, Proc. Natl. Acad. Sci. U.S.A. 86: 6077-6081, 1989, which is herein incorporated by reference); and purified transcription factors (Debs, J. Biol. Chem. 265: 10189-10192, 1990), which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Feigner, Proc. Natl. Acad. Sci. U.S.A. 84: 7413-7416, 1987), which is herein incorporated by reference). Other commercially available liposomes include transfectace (DDAB/DOPE) and DOTAP/DOPE (Boehringer now part of Roche).

Other cationic liposomes can be prepared from readily available materials using techniques well known in the art. See, e.g., PCT Publication No. WO 90/11092 (which is herein incorporated by reference) for a description of the synthesis of DOTAP (1,2-bis(oleoyloxy)-3-(trimet-hylammonio)propane) liposomes. Preparation of DOTMA liposomes is explained in the literature, see, e.g., Felgner, Proc. Natl. Acad. Sci. U.S.A. 84: 7413-7417, 1987, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidy, choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with the DOTMA and DOTAP starting materials in appropriate ratios. Methods for making liposomes using these materials are well known in the art.

For example, commercial dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is maintained at 15° C. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUWs), or large unilamellar vesicles (LUVs), with SUVs being preferred. The various liposome-nucleic acid complexes are prepared using methods well known in the art. See, e.g., Straubinger, Methods of Immunology 101: 512-527, 1983, which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LU-s are prepared by a number of methods, well known in the art. Commonly used methods include Ca²⁺-EDTA chelation (Papahadjopoulos, Biochim. Biophys. Acta 394: 483, 1975; Wilson, Cell 17: 77, 1979; ether injection (Deamer, Biochim. Biophys. Acta 443: 629, 1976; Ostro, Biocheni. Biophys. Res. Comm. 76:836, 1977; Fraley, Proc. Natl. Acad. Sci. U.S.A. 76: 3348, 1979; detergent dialysis (Enoch, Proc. Natl. Acad. Sci. U.S.A. 76: 145, 1979; and reverse-phase evaporation (REV) (Fraley, J. Biol. Chem. 255: 10431, 1980; Szoka and Papahadjopoulos, Proc. Natl. Acad. Sci. U.S.A. 75: 145, 1978; and Schaefer-Ridder, Science 215: 166, 1982), which are herein incorporated by reference.

Generally, the ratio of DNA to liposomes will be from about 10:1 to about 1:10. Preferably, the ratio will be from about 5:1 to about 1:5. More preferably, the ratio will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication no. WO 94/9469 (which are herein incorporated by reference) provide methods for delivering DNA-cationic lipid complexes to mammals.

The use of RNA or DNA viral based systems for the delivery of recombinant constructs encoding fusion proteins of the invention can take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of constructs of the invention include, but are not limited to, retrovirus, lentivirus, human foamy virus, adenovirus, adeno-associated virus (AAV), adeno-AAV, and herpes simplex virus vectors for gene transfer. Viral vectors are currently the most efficient and versatile method of gene transfer in target cells and tissues. Integration in the host genome is possible with the retrovirus, human foamy virus, lentivirus, and adeno-associated virus gene transfer methods, often resulting in long-term expression of the inserted transgene. Additionally, high transduction efficiencies have been observed in many different cell types and target tissues.

The tropism of a retrovirus can be altered by incorporating foreign envelope proteins, expanding the potential target population of target cells. Pseudotypes that are well suited for the transduction of human hematopoietic cells can include the envelopes of gibbon ape leukemia virus (GaLV) (Horn, Blood 100: 3960-7, 2002) and endogenous feline leukemia virus (RD114) (Neff, Mol. Ther. 9: 157-9, 2004). Virus production can be achieved using murine or human packaging cell lines. Lentivirus vectors and human foamy virus vectors are retroviral vectors that are able to transduce or infect non-dividing cells and typically produce high viral titers. Selection of a retroviral gene transfer system would therefore depend on the target tissue. Retroviral vectors are generally comprised of cis-acting long terminal repeats with packaging capacity for up to 6-10 kb of foreign sequence. The minimum cis-acting LTRs are sufficient for replication and packaging of the vectors, which are then used to integrate a construct into the target cell to provide permanent transgene expression. Widely used retroviral vectors include those based upon murine leukemia virus (MuLV), Simian Immuno deficiency virus (SIV), human immuno deficiency virus (HIV), human foamy virus, and combinations thereof (see, e.g., Buchscher, J. Virol. 66: 2731-2739, 1992; Johann, J. Virol. 66: 1635-1640, 1992; Sommerfelt, Virol. 176: 58-59, 1990; Wilson, J. Virol. 63: 2374-2378, 1989; Mergia and Heinkelein, Curr Top Microbiol Immunol. 277: 131-59, 2003, Miller, J. Virol. 65: 2220-2224, 1991; PCT/US94/05700).

Methods for using oncoretrovirus, lentivirus, or human foamy virus vectors for transfer of the fusion protein of the present invention into various cell types, including hematopoietic stem cells, are well described in the literature (reviewed in Brenner and Malech, Biochim Biophys Acta. 1640:1-24, 2003). Hematopoietic stem cells can be obtained by isolating mononuclear cells from the bone marrow or from the peripheral blood, the latter most commonly done using leukapheresis. In most cases, collection of peripheral blood mononuclear cells is performed following several days of G-CSF administration, which acts to mobilize stem cells from the bone marrow to the blood. Hematopoietic stem cells can be enriched from mononuclear cell collections using either positive selection systems (most commonly based on the expression of CD34) or negative selection systems, resulting in the depletion of cells expressing lineage specific cell surface markers. Populations enriched in stem cells can then be subjected to gene transfer. In the case of oncoretrovirus vectors, hematopoietic cells undergo a period of “prestimulation”, during which they are cultured in the presence of a combination of growth factors (usually including stem cell factor, IL-6, thrombopoietin, and flt-3 ligand), most commonly for a period of 48 hours. Gene transfer is commonly accomplished by preloading retrovirus supernatant on retronectin-coated dishes, and then culturing the cells in retrovirus supernatant in the presence of the same or similar combination of cytokines as used during the prestimulation step. Cultures in the presence of retroviral supernatant are typically performed over a period of 48 hours, with 2 or more changes of retroviral supernatant during the culture period. In contrast to oncoretroviral vectors, gene transfer using lentivirus or human foamy virus vectors can commonly be performed overnight without added growth factors. The fewer ex vivo manipulations associated with use of lentivirus or human foamy virus vectors can improve the engraftability of hematopoietic stem cells transduced with these vectors.

Transduced hematopoietic stem cells can have an engraftment defect following transplantation. While many myeloablative conditioning regimens have been described, these are encumbered by toxicity, and it is desirable to employ treatments that facilitate the engraftment of transduced hematopoietic stem cells while minimizing toxicity to the patient. A number of attenuated conditioning regimens that facilitate the engraftment of autologous or allogeneic donor stem cells, have been devised (reviewed in Georges and Storb, Int J Hematol. 77:3-14, 2003). These include the administration of fludarabine and low doses of radiation therapy (typically 200 cGy) (Maris and Storb, Immunol Res. 28: 13-24, 2003). Busulfan administration has been used successfully to facilitate the engraftment of transduced autologous hematopoietic stem cells. (Aiuti, Int J Hematol. 77: 3-14, 2003). Additionally, cells can be genetically modified or otherwise treated to facilitate their engraftment, for example by inhibiting the function of the surface membrane protein, CD26 (Christopherson, Science 305: 1000-3, 2004).

In applications where transient expression of the fusion protein of the invention is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with the recombinant constructions, (see, e.g., West, Virology 160: 38-47, 1987; U.S. Pat. No. 4,797,368; WO 93/24641; Kotin, Human Gene Therapy, 5: 793-801, 1994; Muzyczka, J. Clin. Invest. 94: 1351, 1994. Construction of recombinant AAV vectors are described in a number of publications, including U.S. Pat. No. 5,173,414; Tratschin, Mol. Cell. Biol. 1985, 5:3251-3260; Tratschin, Mol. Cell. Biol. 4: 2072-2081, 1984; Hermonat & Muzyczka, Proc. Natl. Acad. Sci. U.S.A. 81: 6466-6470, 1984; and Samulski, J. Virol. 63: 3822-3828, 1989).

The AAV-based expression vector to be used typically includes the 145 nucleotide AAV inverted terminal repeats (ITRs) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is about 4.4 kb. The following are examples of proteins have been expressed using various AAV-based vectors, and a variety of promoter/enhancers: neomycin phosphotransferase, chloramphenicol acetyl transferase, Fanconi's anemia gene, cystic fibrosis transmembrane conductance regulator, and granulocyte macrophage colony-stimulating factor (see Table 1 in Kotin, Human Gene Therapy 5: 793, 1994). A transgene incorporating the various constructs of this invention can similarly be included in an AAV-based vector. As an alternative to inclusion of a constitutive promoter such as CMV to drive expression of the recombinant DNA encoding the fusion protein(s), an AAV promoter can be used (ITR itself or AAV p5 (Flotte, J. Biol. Chem. 268: 3781, 1993).

Such a vector can be packaged into AAV virions by reported methods. For example, a human cell line such as 293 can be co-transfected with the AAV-based expression vector and another plasmid containing open reading frames encoding AAV rep and cap under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the transgene) and expression of the viral capsid proteins. This system results in packaging of the transgene DNA into AAV virions (Carter, Current Opinion in Biotechnology 3:533, 1992; Kotin, Human Gene Therapy 5: 793, 1994). Methods to improve the titer of AAV can also be used to express the transgene in an AAV virion. Such strategies include, but are not limited to: stable expression of the ITR-flanked transgene in a cell line followed by transfection with a second plasmid to direct viral packaging; use of a cell line that expresses AAV proteins inducibly, such as temperature-sensitive inducible expression or pharmacologically inducible expression. Additionally, the efficiency of AAV transduction can be increased by treating the cells with an agent that facilitates the conversion of the single stranded form to the double stranded form, as described in Wilson, et al. WO96/39530. AAV vectors have been used to direct homologous recombination so that genes can be modified at their endogenous loci (Hirata, Nat Biotechnol. 20: 735-8, 2002). Using this or other approaches for homologous recombination, novel proteins can be generated, for example, by inserting sequences encoding the ligand binding-domain directly adjacent to endogenous genetic sequences encoding a signaling domain of interest. Alternatively, in some embodiments, sequences encoding a desired signaling domain can be inserted adjacent to an endogenously expressed ligand-binding domain.

Concentration and purification of the virus can be achieved by reported methods such as banding in cesium chloride gradients, as was used for the initial report of AAV vector expression in vivo (Flotte, J. Biol. Chem. 268: 3781, 1993) or chromatographic purification, as described in O'Riordan, et al. WO97/08298.

For additional detailed guidance on AAV technology which can be useful in the practice of the subject invention, including methods and materials for the incorporation of a transgene, the propagation and purification of the recombinant AAV vector containing the transgene, and ifs use in transfecting cells and mammals, see e.g., U.S. Pat. Nos. 4,797,368; 5,139,941; 5,173,414; 5,252,479; 5,354,678; 5,436,146; 5,454,935; 5,658,776 and WO 93/24641.

pLASN and MFG-S are examples are retroviral vectors that have been used in clinical trials (Dunbar, Blood 85: 3048-305, 1995; Kohn, Nat. Med. 1: 1017-102, 1995; Malech, Proc. Natl. Acad. Sci. U.S.A. 94: 12133-12138, 1997). PA317/pLASN was the first therapeutic vector used in a gene therapy trial. (Blaese, Science 270: 475-480, 1995). Transduction efficiencies of 50% or greater have been observed for MFG-S packaged vectors. (Ellem, Immunol Immunother. 44: 10-20, 1997; Dranoff, Hum. Gene Ther. 1: 111-2, 1997).

Recombinant adeno-associated virus vectors (rAAV) are promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner, Lancet 351: 1702-3, 1998, Kearns, Gene Ther. 9: 748-55, 1996).

Replication-deficient recombinant adenoviral vectors (Ad) can be engineered such that a transgene replaces the Ad E1a, E1b, and E3 genes; subsequently the replication defective vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman, Hum. Gene Ther. 7: 1083-9, 1998. Additional examples of the use of adenovirus vectors for gene transfer in clinical trials include Rosenecker, Infection 24: 5-10; Sterman, Hum. Gene Ther. 9: 1083-1089, 1998; Welsh, Hum. Gene Ther. 2: 205-18, 1995; Alvarez, Hum. Gene Ther. 5: 597-613, 1997; Topf, Gene Ther. 5: 507-513, 1998; Sterman, Hum. Gene Ther. 7: 1083-1089, 1998.

Packaging cells can be used to form virus particles that are capable of infecting a host cell. Such cells include 293 cells, which package adenovirus, and ψ2 cells or PA317 cells, which package retrovirus. Viral vectors used in gene therapy are usually generated by producer cell lines that package nucleic acid vectors into viral particles. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome, which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment, to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the virus' outer surface. The ligand is chosen to have affinity for a receptor known to be present on the cell type of interest. For example, Han, Proc. Natl. Acad. Sci. U.S.A. 92: 9747-9751, 1995, reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, often after selection for cells that have incorporated the vector.

Ex vivo cell transfection for research or for gene therapy (e.g., via re-infusion of the transfected cells into the host organism) is well known to those of skill in the art. In some embodiments, cells are isolated from the subject organism, transfected with a recombinant construct encoding a fusion protein of the invention, and re-infused back into the subject mammal (e.g., patient). Various cell types suitable for ex vivo transfection are well known to those of skill in the art (see, e.g., Freshney, Culture of Animal Cells, A Manual of Basic Technique (3rd ed. 1994) and the references cited therein for a discussion of how to isolate and culture cells from patients).

Hematopoietic stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Stem cells are isolated for transduction and differentiation using known methods. For example, stem cells are isolated from bone marrow cells by standard immunomagnetic methods using antibodies that deplete differentiated cell types or that positively select for stem cell antigens such as CD34 or CD133. panning the bone marrow cells with antibodies which bind unwanted cells, such as CD4+ and CD8+ (T cells), CD45+(panB cells), GR-1 (granulocytes), and lad (differentiated antigen presenting cells) (see Huntenburg, J. Hematother. 7: 175-83, 1998).

Vectors (e.g., retroviruses, adenoviruses, liposomes, and the like) containing therapeutic nucleic acids can also be administered directly to the organism for transduction of cells in vivo. Alternatively, naked DNA can be administered. In one embodiment, the polynucleotide of the present invention is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, the polynucleotide of the present invention can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The polynucleotide vector constructs used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1/V5, pcDNA3.1, and pRc/CMV2 available from Invitrogen. Other suitable vectors will be readily apparent to the skilled artisan.

Any strong promoter known to those skilled in the art can be used for driving the expression of the polynucleotide sequence. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the β-actin promoter; and human growth hormone promoters. The promoter also can be the native promoter for the polynucleotide of the present invention.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue (including synovial membrane, joint capsule, and perichondrium). Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels or the intraarticular space within synovial joints. Delivery to the interstitial space of muscle tissue is preferred for the reasons discussed below. They can be conveniently delivered by injection into the tissues comprising these cells. They are preferably delivered to and expressed in persistent, non-dividing cells that are differentiated, although delivery and expression can be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and can depend on the condition being treated and the route of administration.

The preferred route of administration is by the parenteral route of injection into the interstitial space of tissues. However, other parenteral routes can also be used, such as, inhalation of an aerosol formulation particularly for delivery to lungs or bronchial tissues, throat, or mucous membranes of the nose. In addition, naked DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

The constructs can also be delivered with delivery vehicles such as viral sequences, viral particles, liposome formulations, lipofectin, precipitating agents, and the like. Such methods of delivery are known in the art.

Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Generally, the DNA or viral particles are transferred to a biologically compatible solution or pharmaceutically acceptable delivery vehicle, such as sterile saline, or other aqueous or non-aqueous isotonic sterile injection solutions or suspensions, numerous examples of which are well-known in the art, including Ringer's, phosphate buffered saline, or other similar vehicles.

Preferably, the DNA or recombinant virus is administered in sufficient amounts to transfect or transduce cells at a level providing therapeutic benefit without undue adverse effects.

### 26. Formulation and Administration of Pharmaceutical Compositions

The nucleic acids, peptides and polypeptides of the invention can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives that are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of nucleic acids, peptides or polypeptides of the invention are dissolved in a pharmaceutically acceptable carrier, e.g., an aqueous carrier if the composition is water-soluble. Examples of aqueous solutions that can be used in formulations for enteral, parenteral, or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents or stabilizers. For example, the solution can contain sodium acetate, sodium lactate, sodium chloride, potassium chloride, calcium chloride, sorbitan monolaurate, or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable, or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, or ethanol.

Nucleic acids, peptides or polypeptides of the invention, when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix, Pharm Res. 13: 1760-1764, 1996; Samanen, J. Pharm. Pharmacol. 48: 119-135, 1996; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. (See, e.g., Sayani, Crit. Rev. Ther. Drug Carrier Syst. 13: 85-184, 1996.) For topical, transdermal administration, the agents are formulated into ointments, creams, salves, powders and gels. Transdermal delivery systems can also include, e.g., patches.

The nucleic acids, peptides, or polypeptides of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention. (See, e.g., Putney, Nat. Biotechnol. 16: 153-157, 1998).

For inhalation, the nucleic acids, peptides or polypeptides of the invention can be delivered using any system known in the art, including dry powder aerosols, liquid delivery systems, air jet nebulizers, propellant systems, and the like. See, e.g., Patton, Biotechniques 16: 141-143, 1998; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

The nucleic acids, peptides or polypeptides of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS. (See e.g., Gurun, Anesth Analg. 85: 317-323, 1997). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's. (See also, Bai, J. Neuroimmunol. 80: 65-75, 1997; Warren, J. Neurol. Sci. 152: 31-38, 1997; Tonegawa, J. Exp. Med. 186: 507-515, 1997.)

In one aspect, the pharmaceutical formulations comprising nucleic acids, peptides or polypeptides of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water-soluble nucleic acids, peptides or polypeptides of the invention have been attached to the surface of the monolayer or bilayer. For example, peptides can be attached to hydrazide-PEG-(distearoylphosphatidyl)ethanolamine-containing liposomes. (See, e.g., Zalipsky Bioconjug. Chem. 6: 705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the nucleic acid, peptides, and/or polypeptides of the invention are incorporated within micelles and/or liposomes. (See, e.g., Suntres, J. Pharm. Pharmacol. 46: 23-28, 1994; Woodle, Pharm. Res. 9: 260-265, 1992). Liposomes and liposomal formulations can be prepared according to standard methods and are also well known in the art. (See, e.g., Remington's; Akimaru, Cytokines Mol. Ther. 1: 197-210, 1995; Alving, Immunol. Rev. 145: 5-31, 1995; Szoka, Ann. Rev. Biophys. Bioeng. 9: 467, 1980, U.S. Pat. Nos. 4,235,871, 4,501,728 and 4,837,028.)

The pharmaceutical compositions are generally formulated as sterile, substantially isotonic, and in full compliance with all Good Manufacturing Practice (GMP) regulations of the U.S. Food and Drug Administration.

### 27. Treatment Regimens and Pharmacokinetics

The pharmaceutical compositions of the invention can be administered in a variety of unit dosage forms depending upon the method of administration. Dosages for typical nucleic acid, peptide and polypeptide pharmaceutical compositions are well known to those of skill in the art. Such dosages are typically advisorial in nature and are adjusted depending on the particular therapeutic context, patient tolerance, and the like. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer Science 249: 1527-1533, 1990.

In therapeutic applications, compositions are administered to a patient suffering from a musculoskeletal disorder to at least partially arrest the condition or a disease and/or its complications. For example, in one aspect, a soluble peptide pharmaceutical composition dosage for intravenous (IV) administration would be about 0.01 mg/hr to about 1.0 mg/hr administered over several hours (typically 1, 3 or 6 hours), which can be repeated for weeks with intermittent cycles. Considerably higher dosages (e.g., ranging up to about 10 mg/mil) can be used, particularly when the drug is administered to a secluded site and not into the blood stream, such as into a body cavity or into a lumen of an organ, e.g., the cerebrospinal fluid (CSF) or a joint space or structure.

The invention provides pharmaceutical compositions comprising one or a combination of antibodies, e.g., antibodies to morphogenic gene products (monoclonal, polyclonal or single chain Fv; intact or binding fragments thereof) or nucleic acid compositions, e.g., antisense oligonucleotides, double stranded RNA oligonucleotides (RNAi) or DNA oligonucleotides (vectors) containing nucleotide sequences encoding for the transcription of shRNA molecules, formulated together with a pharmaceutically acceptable carrier. Some compositions include a combination of multiple (e.g., two or more) monoclonal antibodies or antigen-binding portions thereof of the invention. In some compositions, each of the antibodies or antigen-binding portions thereof of the composition is a monoclonal antibody or a human sequence antibody that binds to a distinct, pre-selected epitope of an antigen.

In prophylactic applications, pharmaceutical compositions or medicaments are administered to a patient susceptible to, or otherwise at risk of a disease or condition (e.g., a musculoskeletal disorder) in an amount sufficient to eliminate or reduce the risk, lessen the severity, or delay the outset of the disease, including biochemical, histologic and/or behavioral symptoms of the disease, its complications, and intermediate pathological manifestations presenting during development of the disease. In therapeutic applications, compositions or medicants are administered to a patient suspected of, or already suffering from such a disease in an amount sufficient to cure, or at least partially arrest, the symptoms of the disease (biochemical, histologic, and/or behavioral), including its complications and intermediate pathological manifestations in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient immune or other desired response has been achieved. Typically, any response is monitored and repeated dosages are given if the response starts to wane.

### 28. Effective Dosages

Effective doses of the antibody compositions of the present invention, e.g., antibodies to morphogenic gene products (e.g., morphogenic proteins), or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of a musculoskeletal disorder described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Doses need to be titrated to optimize safety and efficacy.

For administration with an antibody or nucleic acid composition, the dose ranges from about 0.0001 to 100 mg/kg, and more usually 0.01 to 5 mg/kg, of the host body weight. For example doses can be 1 mg/kg body weight or 10 mg/kg body weight or within the range of 1-10 mg/kg. An exemplary treatment regime entails administration once per every two weeks or once a month or once every 3 to 6 months. In some methods, two or more monoclonal antibodies with different binding specificities are administered simultaneously, in which case the dose of each antibody administered falls within the ranges indicated. Antibody is usually administered on multiple occasions. Intervals between single dosages (‘dosage’ actually appropriate here) can be weekly, monthly or yearly. Intervals can also be irregular as indicated by measuring blood levels of antibody in the patient. In some methods, dose is adjusted to achieve a plasma antibody concentration of 1-1000 μg/ml and in some methods 25-300 μg/ml. Alternatively, antibody can be administered as a sustained release formulation, in which case less frequent administration is required. Dose and frequency vary depending on the half-life of the antibody in the patient. In general, human antibodies show the longest half-life, followed by humanized antibodies, chimeric antibodies, and nonhuman antibodies. The dose and frequency of administration can vary depending on whether the treatment is prophylactic or therapeutic. In prophylactic applications, a relatively low dose is administered at relatively infrequent intervals over a long period of time. Some patients continue to receive treatment for the rest of their lives. In therapeutic applications, a relatively high dose at relatively short intervals is sometimes required until progression of the disease is reduced or terminated, and preferably until the patient shows partial or complete amelioration of symptoms of disease. Thereafter, the patient can be administered a prophylactic regime.

Doses for nucleic acids range from about 10 ng to 1 g, 100 ng to 100 mg, 1 μg to 10 mg, or 30-300 μg DNA per patient. Doses for infectious viral vectors vary from 10-100, or more, virions per dose.

### 29. Routes of Administration

Antibody compositions for inducing an immune response, e.g., antibodies to morphogenic gene products (e.g., morphogenic proteins), or nucleic acid compositions, e.g., antisense oligonucleoties, double stranded RNA oligonucleotides (RNAi), or DNA oligonucleotides (vectors) containing nucleotide sequences encoding for the transcription of shRNA molecules, for the treatment of treatment of a musculoskeletal disorder described herein, can be administered by parenteral, topical, intravenous, oral, subcutaneous, intraarterial, intracranial, intraperitoneal, intranasal, or intramuscular means for prophylactixis? as inhalants for antibody preparations and/or therapeutic treatment. The most typical route of administration of an immunogenic agent is subcutaneous, although other routes can be equally effective. The next most common route is intramuscular injection. This type of injection is most typically performed in the arm, shoulder, or leg muscles. In some methods, agents are injected directly into a particular tissue, for example intracranial injection or convection-enhanced delivery. Intramuscular injection or intravenous infusion are preferred for administration of antibody. In some methods, particular therapeutic antibodies are delivered directly into the cranium. In some methods, antibodies are administered as a sustained release composition or device, such as a Medipad™ device.

Agents of the invention can optionally be administered in combination with other agents that are at least partly effective in treating various musculoskeletal disorders.

### 30. Formulation

Antibody compositions for inducing an immune response, e.g., antibodies to antibodies to morphogenic gene products (e.g., morphogenic proteins), or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of treatment of a musculoskeletal disorder described herein, are often administered as pharmaceutical compositions comprising an active therapeutic agent, i.e., and a variety of other pharmaceutically acceptable components. See the most recent edition of Remington's Pharmaceutical Science (e.g., 20^{h} ed., Mack Publishing Company, Easton, Pa., 2000). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation, which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990; Hanes, Advanced Drug Delivery Reviews 28: 97-119, 1997. The agents of this invention can be administered in the form of a depot injection or implant preparation, which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations, or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (see Glenn, Nature 391: 851, 1998). Co-administration can be achieved by using the components as a mixture or as linked molecules obtained by chemical crosslinking or expression as a fusion protein.

Alternatively, transdermal delivery can be achieved using a skin patch or using transferosomes. Paul, Eur. J. Immunol. 25: 3521-24, 1995; Cevc, Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### 31. Toxicity

Preferably, a therapeutically effective dose of the antibody compositions or nucleic acid compositions, e.g., antisense oligonucleotides, double stranded RNA oligonucleotides (RNAi), or DNA oligonucleotides (vectors) containing nucleotide sequences encoding for the transcription of shRNA molecules, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Hardman, J. G. L. E. Limbird, and A. G. Gilman, 2001, THE PHARMACOLOGICAL BASIS OF THERAPEUTICS (McGraw-Hill Professional Publishers).

### 32. Kits

For use in diagnostic, research, and therapeutic applications suggested above, kits are also provided by the invention. In the diagnostic and research applications such kits can include any or all of the following: assay reagents, buffers, hCDMP-1 variant nucleic acids or antibodies, hybridization probes and/or primers, antisense polynucleotides, ribozymes, dominant negative hCDMP-1 variant polypeptides or polynucleotides, small molecule inhibitors or activators of hCDMP-1 variants, and the like. A therapeutic product can include sterile saline or another pharmaceutically acceptable emulsion and suspension base as described above.

Accordingly, kits of the present invention can contain any reagent that specifically hybridize to hCDMP-1 variant nucleic acids, e.g., hCDMP-1 variant probes and primers, and hCDMP-1-specific reagents that specifically bind to and/or modulate the activity of a hCDMP-1 variant protein, e.g., hCDMP-1 variant antibodies, hCDMP-1 variant ligands, or other compounds, are used to treat hCDMP-1-associated diseases or conditions. Kits of the present invention can also contain additional agents that can be administered concomitantly with the compounds of the present invention. In addition, kits can contain reagents or other components used to locate CDMP-1, Vg1, or SPC1, 4, or 6 polypeptides, or nucleic acid probes, primers, or other materials that can be used to detect biological activation of CDMP-1 or Vg1. These may include, but are not limited to, specific antibodies or antisera, e.g., to phospho-Smad proteins associated with activation of the polypeptides of the invention and/or PCR primers to detect genes transcribed in response to CDMP-1 or Vg1 signaling.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

### EXEMPLARY EMBODIMENTS

Experimental Procedures

Isolation of XGDF5 cDNA

A primer (5′-TATCATTGTGAAGGGCTTTGTGAGTTCC-3′) designed to a highly conserved region within the mature domain of human, mouse, chicken, and zebrafish GDF5, was used with the SMART™ RACE cDNA Amplification Kit (Clontech) and cDNA obtained from stage 22-24 Xenopus embryos. A 302 bp PCR product was obtained which encoded part of the mature region of Xenopus GDF5, including the stop codon. From this, the full-length Xenopus GDF5 (XGDF5) was isolated using 5′ SMART® RACE cDNA amplification using specific primers and Xenopus cDNA obtained from stage 59 limbs (Accession number AY685227).

Plasmids and Probes

The XGDF5 open reading frame was subcloned into pBluescript (pXGDF5) to generate probes for hybridization in situ and CS2 (CS2XGDF5) for production of capped mRNA for injection experiments using the MEGAscript and mMessage mMachine kits (Ambion), respectively. A point mutation (A→G) was made in CS2XGDF5 using the Quickchange™ Site-Directed Mutagenesis kit (Stratagene) to change the RRKR cleavage site to RRRR, producing CS2XGDF5-R. Using PCR extension, a T7-tag flanked by Earl restriction sites was subcloned into PCR4TOPO (Invitrogen). The T7-tag was subsequently subcloned into CS2XGDF5 and CS2XGDF5-R at a unique Earl site 3′ to the RXXR cleavage site, producing CS2XGDF5-T7 and CS2XGDF5-R-T7.

Xenopus Vg1 was obtained as an IMAGE EST clone (3472800) in pBSRN3 (Research Genetics). It was subcloned into pBluescript (pXVg1) and CS2 (CS2XVg1) as an EcoRI-SpeI fragment encoding the complete open reading frame, 23 bp of 5UTR and 68 bp of 3′UTR. A point mutation (A→G) was made in CS2XVg1 to change the RRKR cleavage site to RRRR, producing CS2XVg1-R. A T7-tag was introduced into CS2XVg1 and CS2XVg1-R by first introducing a SacI and EcoRI site immediately after the RXXR site and then subcloning the SacI-EcoRI T7-tag fragment from CS2XCGDF5 to produce CS2XVg1-T7 and CS2XVg1-R-T7. Xenopus BVg1, containing the pro-region and RXXR site of BMP2 and the mature Vg1 domain was a gift from D. Kessler (University of Pennsylvania). Xenopus Furin was obtained as a 3122 bp full-length Image clone (3397590) encoding 326 bp of 5′-UTR, 2349 bp of ORF and 447 bp 3′-UTR and was subcloned into CS2 as an EcoRI-NotI (blunt-ended) fragment. A near full-length EST for Xenopus SPC4 (Image clone 6865482) was obtained. The missing 211 bp of 5′ sequence was generated using the 5′-SMART™ RACE kit (Clontech) and Xenopus limb cDNA as template. The resulting PCR product was subcloned into PCR-4 TOPO (Invitrogen) and the full-length construct made by subcloning a 3734 bp NdeI-NotI fragment from Image clone 6865482. Xenopus SPC4 was subcloned into CS2 as a 3401 bp PmeI fragment encoding 52 bp of 5′-UTR, 2733 bp of ORF and 616 bp of 3′-UTR. Xenopus SPC6A, obtained as Image clone 4173657, was subcloned into CS2 as a 3114 bp EcoRI-XhoI fragment encoding 332 bp 5′-UTR, 2733 bp of ORF and 49 bp 3′-UTR. The Xenopus SPC7 ORF (2265 bp) was contained in 2556 bp insert in pBluescript (Image clone 3378364). Full-length Xenopus Siamois in CS2, a gift from D. Kessler, was subcloned into PCR4-TOPO for generating RNA probes, as was mouse GDF5, isolated as a 137 lbp PCR fragment encompassing the pro-region using the primers 5′-TCGGCTTTCTCCTTTCAAGAACGA-3′ and 5′-ACAGTCTTGTCATCCTGGCCAGA-3′. Mouse Furin, SPC4, and SPC6 obtained as EST's (Image clones 6492009, 4167960, and 6836178 respectively), were subcloned into pBluescript.

Oocyte Injections and Embryo Manipulations

Enzymatically defolliculated oocytes were injected with up to 50 ng of 5′-Capped mRNAs and cultured in 50 μl of oocyte Ringer's solution (Kay, 1991) for 48 hours in 96 well plates at a density of 5 oocytes per well before harvesting.

Frogs and their embryos were maintained and manipulated using standard methods (Gurdon, 1967; 1977). All embryos were staged according to Nieuwkoop and Faber (Nieuwkoop and Faber, 1967) and Keller (Keller, 1991). mRNA injection experiments were performed by standard procedures as described previously (Moos et al., 1995). Dorsal and ventral blastomeres were identified by size and pigment variations (Nieuwkoop, 1967). Animal cap explants were cultured in 0.5× Marc's Modified Ringer's solution (Sive et al., 2000). mRNAs were injected into a single blastomere at the two cell stage or one dorsal blastomere at the four cell stage. Embryos were ventralized 20 min after fertilization by irradiating vegetal hemispheres with UV light (4×10⁴ μJ/cm²) using an inverted Spectrolinker™ (Spectronics Corp.). mRNAs were injected into single vegetal blastomeres of 4 and 8 cell stage embryos.

Perturbations of axial patterning were quantified by Dorso-Anterior Index (DAI, Kao and Elinson, 1988). Darkfield images of embryos were photographed with low angle oblique illumination and a Zeiss Stemi-6 dissecting microscope.

Immunoblotting

Oocyte media were collected after 18-48 hours post injection, snap frozen on dry ice, and stored at −80° C. until analysis. Oocytes were lysed by sonication on ice in 40 mM Tris base, 10 mM EDTA, 1 mM phenylmethyl sulfonyl fluoride in a volume of 10 μl/oocyte. Extracts were centrifuged at 20,000×g for 5 minutes. Supernatants were extracted with an equal volume of 1, 1, 2-trichlorotrifluoroethane to reduce the vitellogenin content (Evans and Kay, 1991). SDS-PAGE was done with Novex 10% Nu-PAGE gels using the MES buffer system. Immunoblot analysis was performed using the mini-PROTEAN II system (BioRad) and Immobilon™-P PVDF membranes (Millipore). Tagged proteins were detected using T7 tag-HRP conjugated monoclonal antibody (Novagen) and SuperSignal® West Femto Maximum Sensitivity Substrate (Pierce).

RT-PCR

Separate pools of embryos or explants were prepared from at least two different fertilizations for each condition reported. Total RNA was prepared with TriZol™ and treated with DNA-Free™ DNAse removal reagent (Ambion). Reverse transcription was done using Superscript II (Life Technologies) as described by the manufacturer, with 1 μg total RNA per reaction; 2% of the cDNA obtained was used in each PCR. Amplification was performed in 10 μl reactions containing 50 mM TRIS-HCl, pH 8.3, 2 mM MgCl₂, 0.25% bovine albumin, 2.5% Ficoll 400, 5 mM cresol red, 200 μM dNTPs, 0.5 μM each primer, and 0.2 U Advantage® 2 Taq polymerase (Clontech). Each cycle comprised 94° C., 0 seconds; 55° C., 0 seconds; 72° C., 40 seconds; a 1 minute denaturation at 94° C. preceded cycling and a 2 minute extension at 72° C. was included after the final cycle. An Idaho Technologies air thermal cycler was used in all experiments. Optimal cycle numbers and annealing temperatures were determined for each primer set. PCR products were separated on 2% agarose gels in TAE buffer, stained with SYBR Green 1™ (Molecular Probes, Eugene, Oreg.) and scanned using a Molecular Dynamics Fluorimager. PCR analysis was performed at least twice for each cDNA to confirm that the amplifications were reproducible. The Xenopus primers for Histone H4, Brachyury, Cardiac Actin, and N-CAM have been described previously (Hemmati-Brivanlou et al., 1994; Niehrs et al., 1994).

Hybridization In Situ

cRNA probes were produced using MEGAscript T3, T7, or SP6 in Vitro transcription kits (Ambion), incorporating either Digoxigenin or Fluorescein. For whole mount hybridization in situ on Xenopus embryos, procedures outlined by Harland were followed (Harland, 1991), with modifications as described (Moos et al., 1995). Xenopus and mouse paraffin sections (10 μm) were prepared for fluorescent hybridization in situ using a standard protocol (Butler et al., 2001) with the following modifications: Dewaxing was carried out in Clear-Rite 3 (Richard-Allan Scientific, MI). A H₂O₂ step was included (0.5% for 20 min at RT) to remove any endogenous peroxidase activity. Prior to hybridization, sections were incubated for 30 min at 90° C. in 10 mM citrate buffer, pH 6.0 to enhance antigenicity (Zaidi et al., 2000). Hybridization was performed at 60° C. overnight in the presence of 1 μg/ml of each probe. For single-label colorimetric detection, signals were developed using alkaline-phosphatase conjugated antibodies to digoxigenin and BM-Purple (Roche). Control hybridizations with sense probes were negative. For double-label fluorescent detection, probes were labeled with either fluorescein or digoxigenin. An alkaline-phosphatase conjugated anti-fluorescein antibody and a horseradish-peroxidase conjugated anti-digoxigenin antibody (Roche) were used in combination with Fast™ Red (Sigma) and tyramide fluorogenic substrates (Molecular Probes), respectively. Confocal images were obtained using a BioRad Radiance confocal microscope with a krypton-argon and blue diode.

Results

Isolation and Expression of Xenopus GDF5

In previous experiments, dorsal overexpression of mammalian (murine and human) GDF5/CDMP1 in Xenopus embryos produced little or no effect on axial patterning (Dionne et al., 2001; Thomas and Moos, not shown). To determine whether this lack of effect might result from a species-to-species variation, we isolated the Xenopus GDF5 ortholog. A full-length sequence obtained using SMART™-RACE PCR and stage 59 Xenopus limb cDNA as template (Accession number AY685227) was more similar to mammalian CDMP1/GDF5 than to other BMPs (FIG. 1A). In the region corresponding to the mature peptide, the amino acid sequence of the Xenopus clone was 92% identical to the human sequence and 93.6% identical to the chicken sequence. In the remainder of the molecule (the amino-terminal “pro” region), the sequence identities were 48% (Xenopus-human) and 52% (mouse-human; FIG. 1B). RT-PCR analysis of Xenopus GDF5 at various stages during development failed to detect GDF5 mRNA in tadpoles or younger embryos (stages 40 and below), but revealed high levels of expression in developing limbs at stage 59 (FIG. 1C). Hybridization in situ, using Xenopus limb whole mounts, showed Xenopus GDF5 to be localized at the joint interzones (FIG. 1D), consistent with the spatial expression pattern observed in mammals (Storm et al., 1994).

Biological Activity of Xenopus GDF5 and Vg1 Depends on Proteolytic Processing

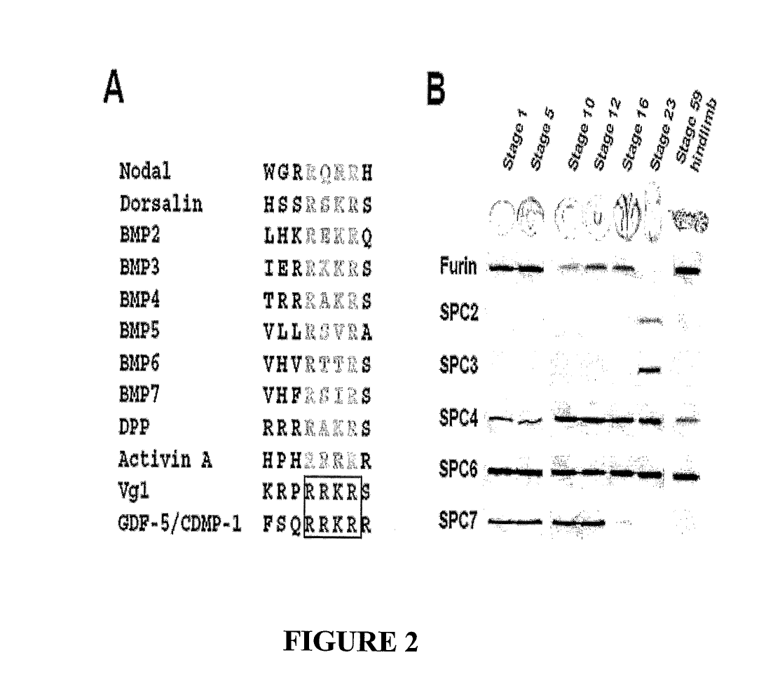

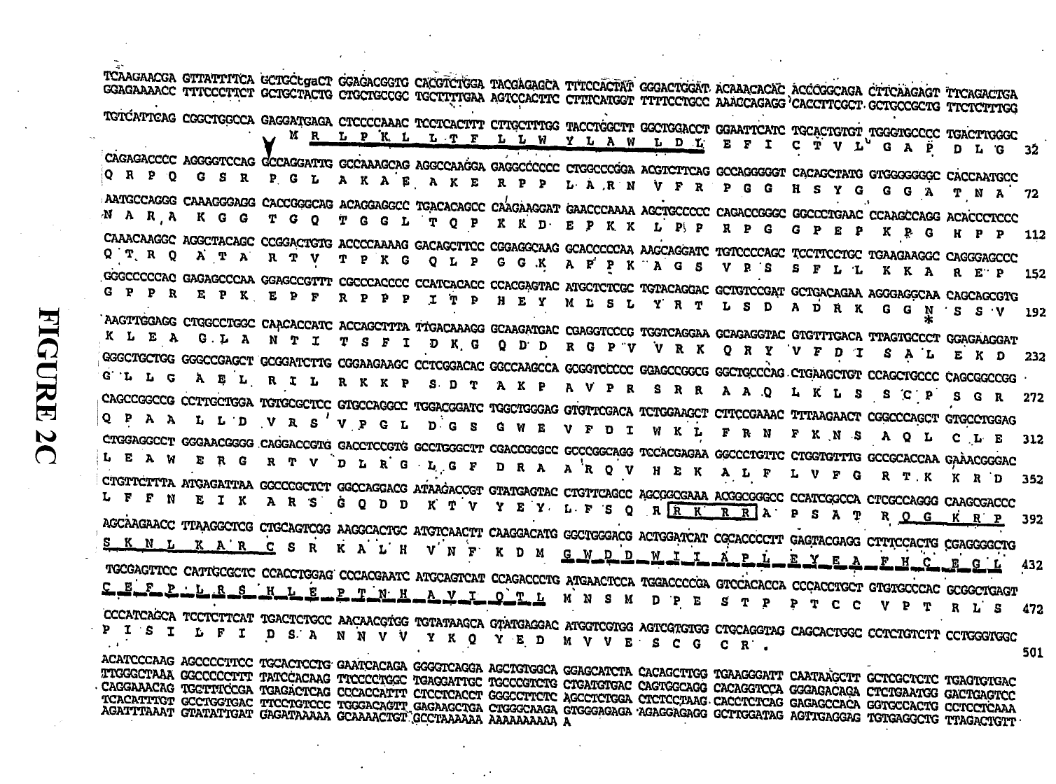

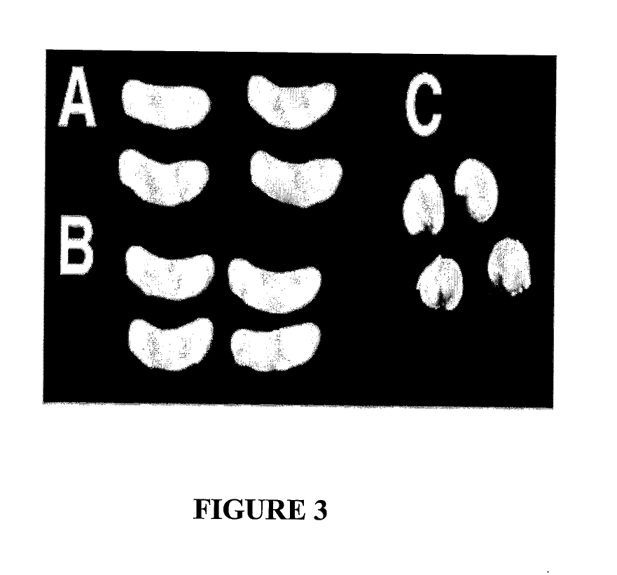

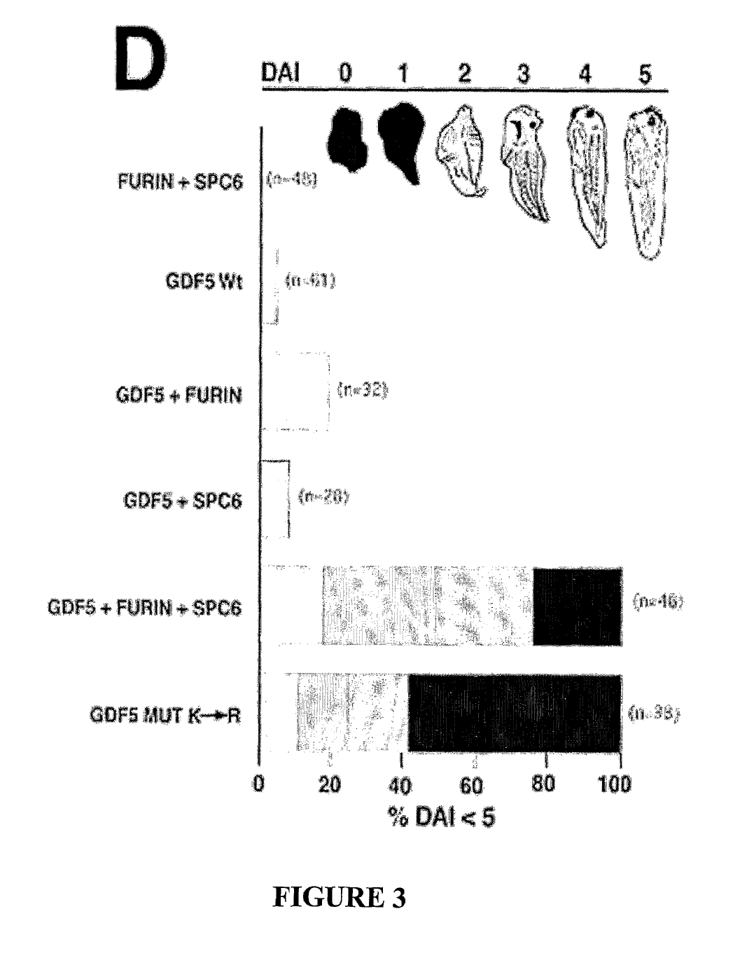

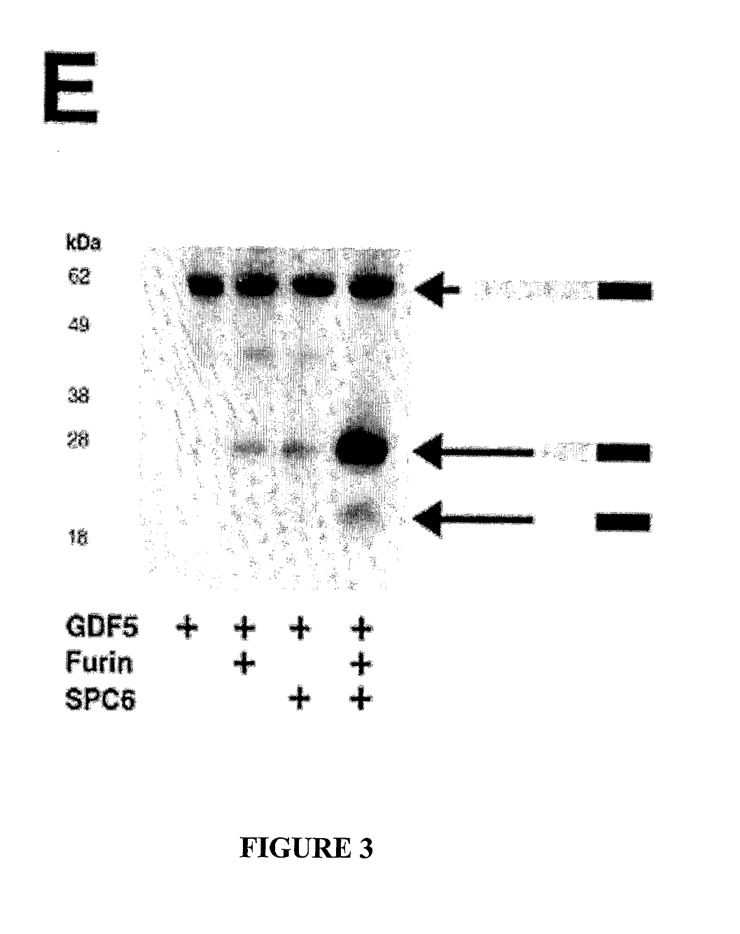

Xenopus GDF5 was overexpressed in Xenopus embryos by injecting up to 100 pg of mRNA into a single blastomere at the two- or four-cell stage with dorsal targeting. Only very mild ventralization (DAI value 4) was observed in 5% of injected embryos, whereas the remainder appeared normal (FIG. 3B). Since this behavior was inconsistent with the biological effects elicited by most other BMPs, we explored possible explanations. All orthologs of CDMP1/GDF5 have the putative proteolytic cleavage recognition sequence RRKR. Of all known BMPs, only CDMP1/GDF5 and Vg1 share this sequence. Since these two proteins are the only BMPs known to have no effect in Xenopus patterning assays, we tested whether modifications to the RRKR sequence resulted in detectable biological activity (FIG. 2A).

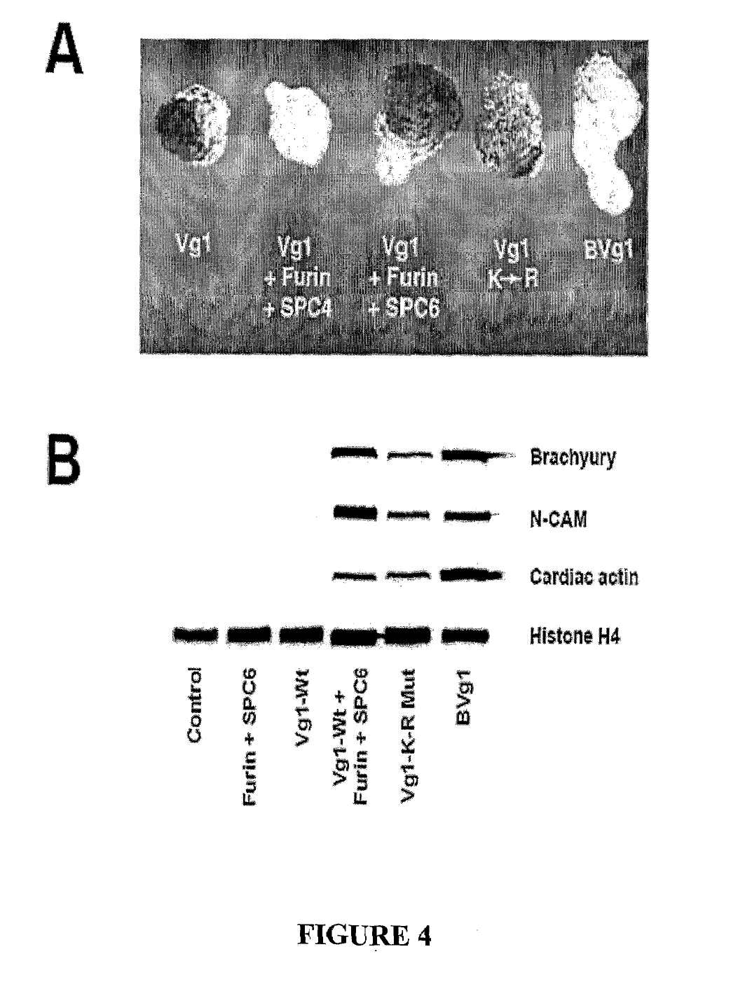

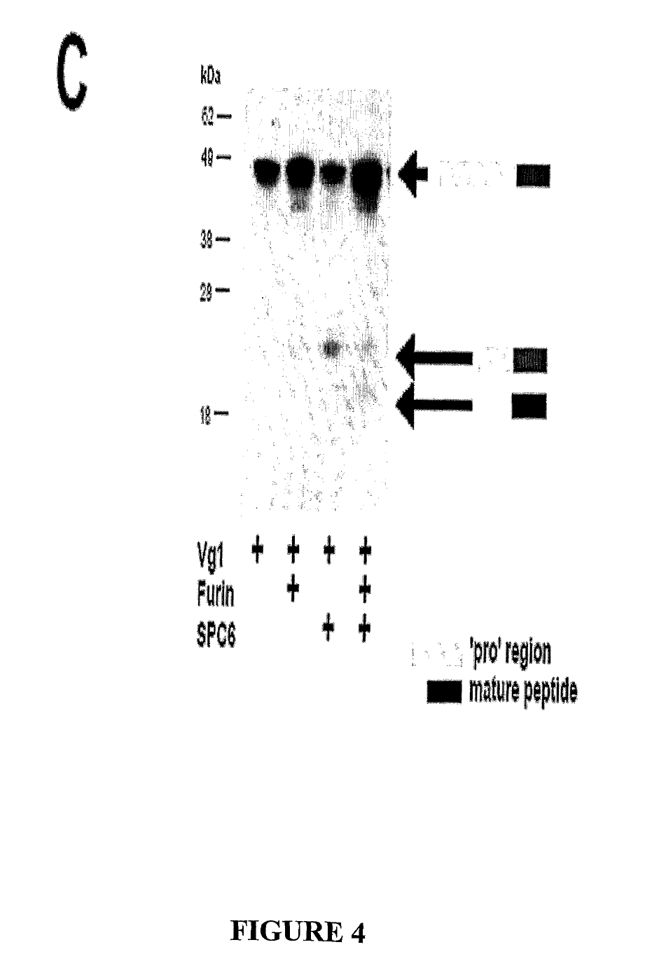

The RXXR sequence for the highly active TGF-β superfamily member Activin (RRRR) differs by only one amino acid (FIG. 2A). We therefore engineered a K→R point mutation into GDF5 and Vg1 to change their cleavage sites to RRRR. We could thus test whether biological inactivity of wild type GDF5 and Vg1 could be explained by the nature of the RXXR site. Dorsal microinjection of Xenopus GDF5 (K→R) into two- and four-cell embryos produced severe ventralization (Average DAI=1.4, FIG. 3C). Overexpression of the Vg1 point mutant in animal cap explants induced mesoderm, as indicated by elongation of the caps and induction of molecular markers characteristic of mesoderm (FIGS. 4A and B). These results support the idea that the RRRR sequence can be acted upon by proteases occurring ubiquitously, but the RRKR sequence cannot.

To determine which SPC(s) could be involved in GDF5 and Vg1 cleavage, orthologs for six of the seven SPCs known to occur in mammals were identified in Xenopus. Developmental stages when Vg1 (stage 10 and earlier) and GDF5 (stage 59 limbs) are expressed were tested for SPC expression. In limbs, RT-PCR demonstrated that Furin (SPC1), SPC4, and SPC6 were present, whereas SPC2, SPC3, and SPC7 were either absent or in very low abundance (FIG. 2B). Furin, SPC4, SPC6 and SPC7 were detected at stages 1, 5, 10, and later stages (FIG. 2B). Using EST clones and Smart™ RACE PCR, full-length Xenopus Furin, SPC4 (Accession number AY685228), SPC6A (Accession number AY685229), and SPC7 were obtained for use in hybridization in situ and microinjection studies. The amino acid sequence identities between human and Xenopus SPC4 and SPC6A were 68% and 76% respectively.

Co-injection of GDF5 message with Furin, SPC4 (not shown), or SPC6 mRNA alone produced mild ventralization (Average DAI=4.8, 4.8, and 4.9 respectively; FIG. 3D) that was significantly less than that observed for GDF5 (K→R) alone (Average DAI=1.4). We next examined whether co-injection of GDF5 with two different SPCs would result in enhanced GDP5 activity. Combinations of either Furin and SPC4 or SPC4 and SPC6 messages without GDF5 did not increase the degree of ventralization above that observed when the SPC mRNAs were injected individually (not shown). In these experiments, ventralization was not enhanced when the amounts of injected SPC mRNA were doubled (results not shown), suggesting the doses of mRNA for the single SPCs were saturating. In contrast, injection of GDF5 message together with the combination of Furin and SPC6 mRNAs produced severe effects (Average DAI=2.4), similar to those observed for the K→R mutant.

Analogous experiments were performed with Vg1. Dorsal blastomeres of four cell embryos were microinjected with mRNAs for Vg1 and various SPCs. Animal caps removed at stage 9 and cultured until stage 24 were analyzed for evidence of mesoderm formation. Explants from Xenopus embryos injected with RNAs encoding either the K→R form of Vg1 or the combination of wild-type Vg1, Furin, and SPC6 were elongated (FIG. 4A), and RT-PCR analysis demonstrated the presence of mesoderm markers (FIG. 4B). Similar results were obtained when wild type Vg1 was co-injected with SPC4 plus SPC6 (not shown). Overexpression of wild type Vg1 alone was ineffective (FIG. 4B).

Release of Mature GDF5 and Vg1 Requires Combinations of SPCs

To confirm biochemically that combinations of proteases were more effective in converting XGDF5 and Vg1 to their mature forms, the growth factors were co-expressed with protease combinations in Xenopus oocytes. For these experiments, T7 epitope tags were introduced as described, so that expression of both unprocessed and mature peptides could be detected by immunoblot analysis. The molecular weights for Xenopus pro-GDF5-T7 (58.5 kDa), pro-Vg1-T7 (45 kDa), mature GDF5-T7 (16.5 kDa), and mature Vg1-T7 (16 kDa) were calculated. The pro-forms of GDF5 (FIG. 3E) and Vg1 (FIG. 4C) could be detected in oocyte supernatants for all treatments. However, XGDF5 and Vg1 mature peptides could be detected only when mRNAs encoding both Furin and SPC6 were co-injected with the growth factor messages (FIG. 3E and FIG. 4C). Similar results were obtained with Vg1 and the combination of SPC4 and SPC6 (not shown). The additional bands observed migrating above the mature forms of both GDF5 and Vg1 are presumably partially processed peptides.

Co-Expression of Growth Factors and SPCs In Vivo

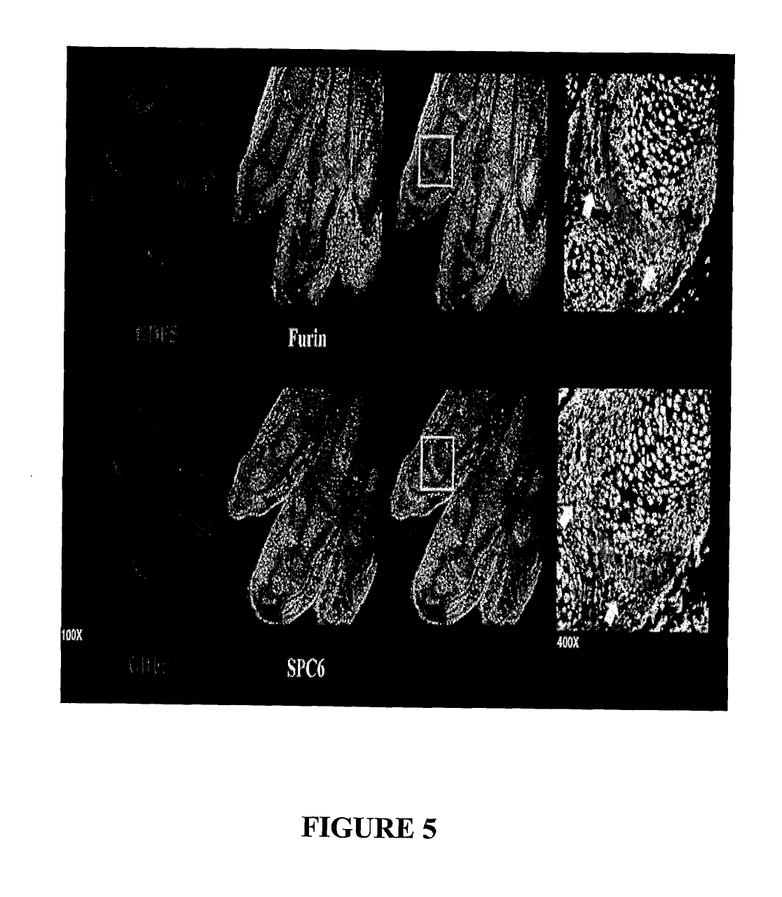

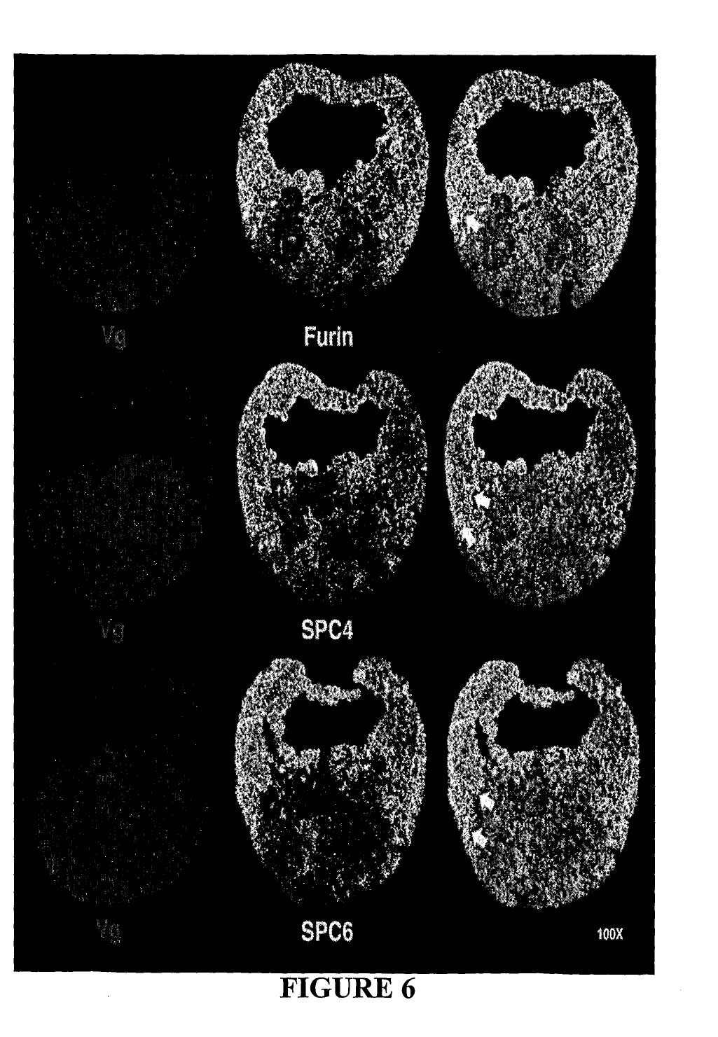

For XGDF5 or Vg1 to be processed in vivo, the expression domains of the SPCs implicated in the experiments described above must overlap those of the growth factors. We compared spatial expression patterns of these genes using double-label fluorescent hybridization in situ. For the GDF5 experiments, we chose paraffin sections of mouse embryo limbs and probes for comparison of GDF5 to Furin, SPC4, and SPC6, since data were technically superior to those obtained using Xenopus limbs. GDF5 and each of the SPCs were analyzed pairwise in serial sections so that expression could be compared directly. In 14.5 and 15.5 dpc mouse embryo limbs, GDF5 was expressed throughout the developing joint interzones, consistent with earlier reports (Storm et al., 1994). Although expression of the SPCs in the digits was widespread (FIG. 5), a discrete region of overlapping expression with GDF5 was observed at the boundary of the developing articular surface (FIG. 5). Analogous studies for Vg1 were conducted on paraffin sections of stage 8 Xenopus embryos. As expected for this stage of development, Vg1 mRNA was detected throughout the vegetal endoderm (FIG. 6). In contrast, Furin, SPC4, and SPC6 were present predominantly in the animal region (FIG. 6). However, overlapping expression was observed in a small number of cells within the dorsal vegetal endoderm (FIG. 6), a location corresponding to the Nieuwkoop center. The dorsal location was confirmed by probing adjacent sections with cRNA for Siamois (not shown), which is expressed at this site (Lemaire et al., 1995). Xenopus SPC7 was detected in distinct animal blastomeres on the dorsal side of the embryo (not shown). Since its expression was far removed from that of Vg1, it was not considered a candidate for Vg1 processing.

Vegetal Injection of Furin and SPC6 mRNA Rescues Anterior Dorsal Structures in UV-Irradiated Xenopus Embryos

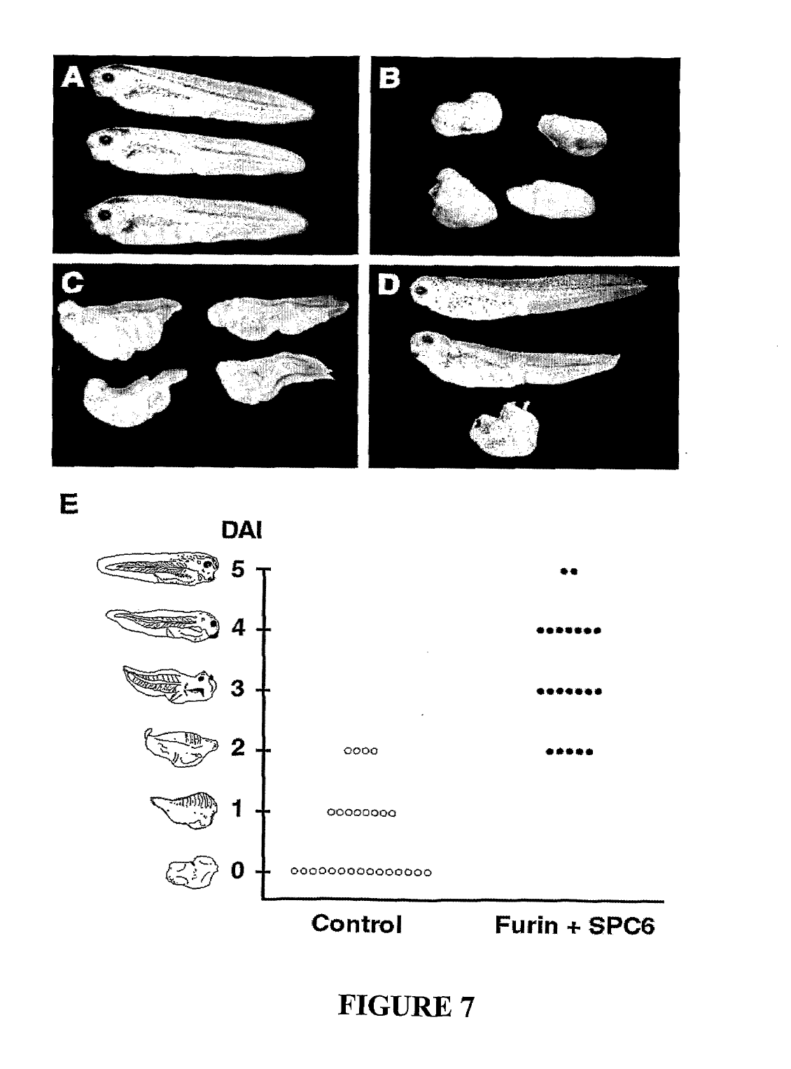

For SPC enzymes to activate Vg1 prior to gastrulation, the necessary mRNAs should be present maternally. Vg1 mRNA is present as a maternal transcript (Weeks and Melton, 1987) but similar data for many of the SPCs has not been reported. RT-PCR revealed that Furin, SPC4, SPC6, and SPC7 were all present as maternal transcripts (FIG. 2B). Following fertilization, cortical rotation is necessary for the activation of Vg1 (Thomsen and Melton, 1993). Perturbation of cortical rotation by exposure of fertilized eggs to UV light results in complete ventralization of resulting embryos, which can be rescued by injection of BVg1 (Thomsen and Melton, 1993). The spatial expression patterns of the SPCs and Vg1 (shown in FIG. 6) suggested that cortical rotation could produce discrete overlap of Furin/SPC4/6 and Vg1 expression at the presumptive Nieuwkoop center. To test this hypothesis, we injected mRNAs encoding Furin and SPC4 or SPC6 into single blastomeres of UV-irradiated 4 and 8 cell stage Xenopus embryos. UV-treated embryos were completely ventralized (FIG. 7B), whereas those injected with mRNAs for Furin and SPC6 (or SPC4, not shown) showed either partial (DAI=2-3) or complete rescue (DAI=4-5) of dorsal axial structures (FIGS. 7C and D). The extent of UV rescue was time and injection site dependent. Optimal rescue was observed when SPCs were injected into a vegetal blastomere of late 4-cell stage embryos (FIG. 7D). Vegetal injection at the mid to late 8 cell stage was less effective (FIG. 7C).

Discussion

Nearly all BMPs tested to date strongly perturb embryonic patterning when overexpressed in Xenopus embryos. In particular, GDF6, which is 78% identical to GDF5 in amino acid sequence in the mature region of the protein, ventralizes zebrafish embryos (Goutel et al., 2000) and promotes epidermis formation while inhibiting formation of neural tissue in Xenopus animal cap explants (Chang and Hemmati-Brivanlou, 1999). We therefore sought to examine the transduction of CDMP1/GDF5 using Xenopus as an assay system. Consistent with an earlier report (Dionne et al., 2001), however, we found that injection of the wild type mRNA produced no effect in conventional Xenopus patterning assays. Of the BMPs tested so far, only one other, Vg1, displays similar lack of biological activity, thought to be due to an unusual requirement for proteolytic processing (Thomsen and Melton, 1993). The finding that COS cells transfected with GDF5 do not process the protein efficiently (Thomas et al., 1997) suggested similarly stringent constraints on GDF5 proteolytic processing. All known GDF5 orthologs have an invariant RRKR sequence within the putative proteolytic processing site (FIG. 2A) identical with that of Vg1. We therefore evaluated the possibility that this feature, common to both proteins, might render them refractory to processing by ubiquitous proteases.

When overexpressed dorsally, the GDF5 K→R mutant ventralized Xenopus embryos dramatically, lending support to the notion that lack of processing might explain the inactivity of wild-type GDF5. Overexpression of wild-type GDF5 with single SPCs (Furin, SPC4, or SPC6) produced only mild ventralization when compared with the K→R mutant, even when high SPC mRNA doses (1 ng) were used. In contrast, the combination of Furin and SPC6 was synergistic, producing ventralization comparable to that obtained with the K→R mutant at much lower total mRNA doses than those used to test single SPCs. The combination of Furin with SPC4 was ineffective, a finding consistent with apparent normal joint development in mice made hypomorphic for SPC4 by targeted deletion (Constam and Robertson, 2000).

The results with GDF5 prompted us to test whether the similarity of the proteolytic cleavage site in Vg1 prevents widespread expression of its effect (Thomsen and Melton, 1993). Characteristic anatomical changes and induction of mesodermal markers in explants overexpressing wild type Vg1 and SPC combinations implicate the presumed proteolytic cleavage site directly, confirming and refining these earlier predictions. Our results suggest that the specific cleavage site shared by Vg1 and GDF5 might confer an especially stringent constraint on cleavage of these growth factors to their mature forms in vivo. This conclusion is supported further by biochemical confirmation that that two SPCs are required to facilitate proteolytic processing of GDF5 or Vg1 (FIGS. 3E and 4C)). Our data do not exclude additional contributions by the N-terminal “pro” domain to processing and secretion of these proteins.

We used dual-label hybridization iii situ to evaluate the proximity of the growth factors and SPCs. Coexpression of GDF5, SPC6, and Furin in narrow zones near the developing joint surfaces is consistent with this requirement (FIG. 5). Consequently, mature, active GDF5 is likely to be secreted only by chondrocytes within the co-localization region at the boundary of the joint interzone. This model would predict that GDF5 expressed within the remainder of the joint interzone will be the non-processed inactive form.

The stripe-like expression pattern of GDF5 at joint interzones resembles that of wingless or decapentaplegic in Drosophila, which serve to organize compartment boundaries (Settle et al. 2003). A similar role can exist for GDF5 in establishing boundaries between developing skeletal elements (Edwards and Francis-West, 2001; Settle et al., 2003). In this regard, the joint interzone can be viewed as a signaling center, regulating chondrocyte proliferation and differentiation and orchestrating joint formation. Overexpression of BMP activity in developing cartilage (Duprez et al., 1996; Tsumaki et al., 1999) or absence of BMP inhibition (Brunet et al., 1998) results in increased chondrocyte differentiation and ablated joint formation. Consequently, it appears crucial to inhibit BMP action within developing cartilage anlagen for normal joint formation to occur. Accordingly, two different BMP antagonists, noggin and chordin, are both expressed within the joint interzone (Brunet et al., 1998; Pathi et al., 1999). Our data suggest that an additional inhibitor of BMP activity is present in the joint interzone: unprocessed CDMP1/GDF5. Expression of CDMP1/GDF5 in the absence of the SPCs required for processing and secretion will result in intracellular accumulation of unprocessed peptide. Previous experiments have demonstrated that unprocessed human CDMP1 is able to heterodimerize with other BMPs, including BMP2 and BMP4, inhibiting their processing and secretion (Thomas et al., 1997). Thus, intra-articular expression of CDMP1/GDF5, but not Furin and SPC6, provides an additional mechanism to block the action of BMPs that would otherwise promote ossification within the joint space. A second requirement for joint formation is that the cartilage elements at either side of the developing joint continue to elongate. Restricting CDMP1/GDF5 processing to the joint interzone boundary (FIG. 5), meets this condition by allowing the activated growth factor to modulate chondrocyte growth and differentiation, thus specifying the joint surface (Francis-West et al., Development 126: 1305-1315, 1999).

Current models of gastrulation call for the release of mesoderm-inducing activity from cells located in the dorsal vegetal endoderm, (for reviews see De Robertis et al., Nat. Rev. Geizet. 1: 171-181, 2000; Moon and Kimelman, Bioessays 20: 536-545, 1998). Over ten years ago, localized proteolytic processing of Vg1 was proposed to direct formation of the Nieuwkoop center (Thomsen and Melton Cell, 74: 433-441, 1993), but no mechanism for such restricted processing has been reported. Overlapping expression of Vg1, SPC4, SPC6, and Furin (FIG. 6), in a discrete region of cells within the dorsal vegetal endoderm, provides a possible molecular definition of the Nieuwkoop center. This model suggests a mechanism for release of mature Vg1 in an anatomically discrete region of the embryo, enabling it to act dorsally in creation of the Spemann organizer. It is also consistent with a possible role for cortical rotation in the activation of mature Vg1 (Thomsen and Melton, 1993; Moon and Kimelman, 1998). Cortical rotation could create a small zone in which Vg1 expression overlaps that of the SPCs, resulting in localized expression of the mature protein. This hypothesis is supported by our finding that injection of SPC mRNAs could rescue a complete dorsal axis in embryos where cortical rotation was blocked by treating with UV-irradiation (FIG. 7). In the absence of cortical rotation, expression of SPCs would not overlap that of Vg1. Vg1 would not be processed, a Nieuwkoop center would not form, and consequently dorsal axial structures would not develop. Ectopic expression of SPC enzymes in vegetal blastomeres of UV-irradiated embryos would process Vg1 to the mature form, thereby reconstituting the functional equivalent of a Nieuwkoop center that can completely rescue the UV phenotype.

We are also able to address the outstanding question of why, unlike many other BMPs, analyses of COS cells transfected with CDMP1/GDF5 (Thomas et al., 1997; Everman et al., 2002) and Xenopus embryos for Vg1 (Tannahill and Melton, 1989) demonstrated both growth factors to be present primarily in the unprocessed form. These observations can be explained by the stringent requirement for two different SPCs co-expressed with the growth factors to expedite their processing. In vivo, we have demonstrated the limited overlap of the growth factors with their respective proteases. In vitro, processing efficiency in transfection experiments will depend on whether the host cells co-express the necessary combination of SPCs.

The synergistic effect of two different SPCs on GDF5 and Vg1 remains to be explained. Our data are consistent with the concept of there being sequential cleavage at two different sites, as described for BMP4 (Cui et al., 2001; FIGS. 3 and 4), but do not account for the observation that the K→R point mutants are processed into biologically active molecules by ubiquitous SPCs. One possibility is that a characteristic higher-order structure associated with the RRKR sequence imposes specific requirements for proteolytic processing of the GDF5 or Vg1 dimer that can be met only by combinations of SPCs. The precise mechanistic details of this process will be of interest to evaluate in the future.

In summary, critical events in patterning of both the joints and body axis appear to be controlled in part by proteolytic processing of key growth factors. Tightly restricted overlap in the expression domains of the growth factors and proteases can thus create sharply limited zones of effect. This phenomenon represents another way in which limb/joint patterning mechanisms parallel those that define the body axis. Our findings also reaffirm the principle that important biological signals often do not act in isolation. Increasingly complex strategies to deliver instructive signaling molecules for therapeutic purposes—for example via gene therapy vectors—will need to address the role of any critical trans acting factors, which can or can not be expressed at the proposed site of administration.

TABLE 2
Exemplary Sequences

Human CDMP-1 (wild type; Accession Number U13660)

(SEQ ID NO: 1)

atgagactccccaaactcctcactttcttgctttggtacctggcttggctggacctggaattcatctgcactgtgttgggtgcccctgacttgggc
cagagaccccaggggtccaggccaggattggccaaagcagaggccaaggagaggccccccctggcccggaacgtcttcaggccagg
gggtcacagctatggtgggggggccaccaatgccaatgccagggcaaagggaggcaccgggcagacaggaggcctgacacagccca
agaaggatgaacccaaaaagctgccccccagaccgggcggccctgaacccaagccaggacaccctccccaaacaaggcaggctacag
cccggactgtgaccccaaaaggacagcttcccggaggcaaggcacccccaaaagcaggatctgtccccagctccttcctgctgaagaag
gccagggagcccgggcccccacgagagcccaaggagccgtttcgcccaccccccatcacaccccacgagtacatgctctcgctgtaca
ggacgctgtccgatgctgacagaaagggaggcaacagcagcgtgaagttggaggctggcctggccaacaccatcaccagctttattgac
aaagggcaagatgaccgaggtcccgtggtcaggaagcagaggtacgtgtttgacattagtgccctggagaaggatgggctgctgggggc
cgagctgcggatcttgcggaagaagccctcggacacggccaagccagcggtcccccggagccggcgggctgcccagctgaagctgtc
cagctgccccagcggccggcagccggccgccttgctggatgtgcgctccgtgccaggcctggacggatctggctgggaggtgttcgaca
tctggaagctcttccgaaactttaagaactcggcccagctgtgcctggagctggaggcctgggaacggggcaggaccgtggacctccgtg
gcctgggcttcgaccgcgccgcccggcaggtccacgagaaggccctgttcctggtgtttggccgcaccaagaaacgggacctgttctttaa
tgagattaaggcccgctctggccaggacgataagaccgtgtatgagtacctgttcagccagcagcgaaaacggcgggccccatcggcca
ctcgccagggcaagcgacccagcaagaaccttaaggctcgctgcagtcggaaggcactgcatgtcaacttcaaggacatgggctgggac
gactggatcatcgcaccccttgagtacgaggctttccactgcgaggggctgtgcgagttcccattgcgctcccacctggagcccacgaatc
atgcagtcatccagaccctgatgaactccatggaccccgagtccacaccacccacctgctgtgtgcccacgcggctgagtcccatcagcat
cctcttcattgactctgccaacaacgtggtgtataagcagtatgaggacatggtcgtggagtcgtgtggctgcaggtag
The nucleotides in bold font correspond to the putative proteolytic processing site.
Human CDMP-1 predicted amino acid sequence (wild type)

(SEQ ID NO: 2)

MRLPKLLTFLLWYLAWLDLEFICTVLGAPDLGQRPQGSRPGLAKAEAKERPPLARNVFR
PGGHSYGGGATNANARAKGGTGQTGGLTQPKKDEPKKLPPRPGGPEPKPGHPPQTRQA
TARTVTPKGQLPGGKAPPKAGSVPSSFLLKKAREPGPPREPKEPFRPPPITPHEYMLSLYR
TLSDADRKGGNSSVKLEAGLANTITSFIDKGQDDRGPVVRKQRYVFDISALEKDGLLGA
ELRILRKKPSDTAKPAVPRSRRAAQLKLSSCPSGRQPAALLDVRSVPGLDGSGWEVFDI
WKLFRNFKNSAQLCLELEAWERGRTVDLRGLGFDRAARQVHEKALFLVFGRTKKRDL
FFNEIKARSGQDDKTVYEYLFSQRRKRRAPSATRQGKPSKNLKARCSRKALHVNFKD
MGWDDWIIAPLEYEAFHCEGLCEFPLRSHLEPTNHAVIQTLMNSMDPESTPPTCCVPTRL
SPISILFIDSANNVVYKQYEDMVVESCGCR
The amino acid residues (RXXR) in bold font correspond to the putative
proteolytic processing site.
Human CDMP-1 K→R mutant

(SEQ ID NO: 3)

atgagactccccaaactcctcactttcttgctttggtacctggcttggctggacctggaattcatctgcactgtgttgggtgcccctgacttgggc
cagagaccccaggggtccaggccaggattggccaaagcagaggccaaggagaggccccccctggcccggaacgtcttcaggccagg
gggtcacagctatggtgggggggccaccaatgccaatgccagggcaaagggaggcaccgggcagacaggaggcctgacacagccca
agaaggatgaacccaaaaagctgccccccagaccgggcggccctgaacccaagccaggacaccctccccaaacaaggcaggctacag
cccggactgtgaccccaaaaggacagcttcccggaggcaaggcacccccaaaagcaggatctgtccccagctccttcctgctgaagaag
gccagggagcccgggcccccacgagagcccaaggagccgtttcgcccaccccccatcacaccccacgagtacatgctctcgctgtaca
ggacgctgtccgatgctgacagaaagggaggcaacagcagcgtgaagttggaggctggcctggccaacaccatcaccagctttattgac
aaagggcaagatgaccgaggtcccgtggtcaggaagcagaggtacgtgtttgacattagtgccctggagaaggatgggctgctgggggc
cgagctgcggatcttgcggaagaagccctcggacacggccaagccagcggtcccccggagccggcgggctgcccagctgaagctgtc
cagctgccccagcggccggcagccggccgccttgctggatgtgcgctccgtgccaggcctggacggatctggctgggaggtgttcgaca
tctggaagctcttccgaaactttaagaactcggcccagctgtgcctggagctggaggcctgggaacggggcaggaccgtggacctccgtg
gcctgggcttcgaccgcgccgcccggcaggtccacgagaaggccctgttcctggtgtttggccgcaccaagaaacgggacctgttctttaa
tgagattaaggcccgctctggccaggacgataagaccgtgtatgagtacctgttcagccagcggcgnnnacggcgggccccatcggcc
actcgccagggcaagcgacccagcaagaaccttaaggctcgctgcagtcggaaggcactgcatgtcaacttcaaggacatgggctggga
cgactggatcatcgcaccccttgagtacgaggctttccactgcgaggggctgtgcgagttcccattgcgctcccacctggagcccacgaat
catgcagtcatccagaccctgatgaactccatggaccccgagtccacaccacccacctgctgtgtgcccacgcggctgagtcccatcagca
tcctcttcattgactctgccaacaacgtggtgtataagcagtatgaggacatggtcgtggagtcgtgtggctgcaggtag
note:
the nnn nucleotide triplet (in bold font) represents the K→R mutant, where nnn can be
represented by the following DNA non-sense triplets for Arg: CGT, CGC, CGA, CGG, AGA, or
AGG;”); Arg can also be represented by the following nucleotide triplets: DNA sense triplet:
GCA, GCG, GCT, GCC, TCT, TCC; RNA codons: CGU, CGC, CGA, CGG, AGA, AGG.
CDMP-1 K→R mutant

(SEQ ID NO: 4)

MRLPKLLTFLLWYLAWLDLEFICTVLGAPDLGQRPQGSRPGLAKAEAKERPPLARNVFR
PGGHSYGGGATNANARAKGGTGQTGGLTQPKKDEPKKLPPRPGGPEPKPGHPPQTRQA
TARTVTPKGQLPGGKAPPKAGSVPSSFLLKKAREPGPPREPKEPFRPPPITPHEYMLSLYR
TLSDADRKGGNSSVKLEAGLANTITSFIDKGQDDRGPVVRKQRYVFDISALEKDGLLGA
ELRILRKKPSDTAKPAVPRSRRAAQLKLSSCPSGRQPAALLDVRSVPGLDGSGWEVFDI
WKLFRNFKNSAQLCLELEAWERGRTVDLRGLGFDRAARQVHEKALFLVFGRTKKRDL
FFNEIKARSGQDDKTVYEYLFSQRRXRRAPSATRQGKRPSKNLKARCSRKALHVNFKD
MGWDDWIIAPLEYEAFHCEGLCEFPLRSHLEPTNHAVIQTLMNSMDPESTPPTCCVPTRL
SPISILFIDSANNVVYKQYEDMVVESCGCR
The K→R mutation is indicated by the X (bold font) above where X
is Arginine (symbol: “R”; abbreviated “Arg.”
GDF5
GDF5 cDNA (Accession Number AB019005)

(SEQ ID NO: 5)

gtcgactcgatcactttatgcaatttaattttatttatttttttagtagagacagggtttcaccatgttagccaggatggtcttgatctcctgacctcgt
gatccacctgcctcggcctcccaaagtgctgggattacaggcgtgagcactgcgcctggccgcaatttacttcattgaatctccaacaagag
tcctgtgaggtaagcactatcgttatcactgtttagagtttcagaggggtttagaggcttcccaagatcacacaatacataaatagcagaacca
agcttcaaaaccaggtctgtttgtctccagaatccttgctctttatcaagccacgtacagagacattgtggttgttcaactcattcatttattcactct
ctgagcttacaaaatgcttaagaagtggcaagacaattcttcccttcaagaaacttagagtctaatgggaaaggcaggttatgtccacaaataa
ctacacctcaaagtagaaaatgatgatttttgtcaataagggccagttagagattaatttattcatataacacgtactgagagctgctgtgggctg
ggcctctgccaagcactgggtataattcaaagataaatatggcacagtctagtcataatctaatgggagagacaggtatgtaaagaaattatta
tagttataaggaattaagttatgattttaaaatatgacaaaatacagagtggaagaaacaaactgtgttgggatagggggcagaggcagaga
ctgaataaactttcagctgccaaaaaatgtaaaagagcacagaaaatgtgtgatggcagctcagggggagtgtgcattttctggaggatgca
agaaggcttcatggaggaggtggcacgtgggttggcacttgtttgatgggtagcttttagtagagggttgaaccagagagagcctggaggc
ctagaagccagttataggggagaatcatagcgaggcaagcaatgaggaggacagggcagtgacagaggtggtgaggagggagtgaag
ggagggatgccagagaggactggttcagtgggaacaagtctgtttttaatttttttattttattaatattatttatttacttattttgtgtgtgtgtgatg
gactctagctcgtcacctaggctggagtgcagtggtgcgatctcggctcactgcaacctttgcctccacacccggctaattttttgtatttttgg
tagagatgcctgcaatcccagcactttgagaggccaaggtgggtggatcacttgaggtcaggagctcgagaccagactggccaacatggt
gaaactccgtctctactaaaaatacaaaaaatagataggcatggtggtgtgcacctgtagtcccagctactcgagaggctgaggcaggaga
atcgtttgaacccaggaggtggagattacagtgagccaagatcgcaccactgcactccagcctaggtgacagagtgagactctgtctcaaa
aaaaaaaaaaaaaaaaaaaaaaaagaaggaagcctatctgactattgcttctcccccgccatccttcttacagcgtgaaaagtgttgttgtag
agaaatgtacagaaggggtaactggctctgtctggggacgaggaaaggcttcccgaaggaggtaaattttcagctaaacttctcaggatgta
gagtgcttcctccagttgtgggagagagagaggaggatatttcaggtgaagcgaacagctttccccgctgaccctgtgttaagttgcccagg
ggctctacggcgaaggttccccagaggaagggatcttccatacttcaatgaggcactttaagaaacccttgagttcagctggaattcagacct
tccagagctaccagaaaaacatcatgtgggaaattgtgcccggcgcggtgtcacgcctgtaatcccagcactttgggaggccgaggcggg
cggatcacgaggtcaggagatcgagaccaacctggctaacatggtgaaaccccatctctactacaaacgtaaaaaatcagccaggtgtggt
ggcaggggcctgtagtcccagctactggggaggctgaggcaggggaatggcgtgaacctgggaggcagagattgcagtgagctgagat
catgccactgcactcgacagaggcgaactccgtctcaaaaaaaaaaaaaaaaaaagcgtcatgtgggaaatggcttttccagcctcctgta
ggggccgctgctgccccagactcagccagtcttgtctaagaaaactcagaggacgtctctgctggggtggtggtggggtacaccctggac
ctgccgccatccaaggagtgaacttaagggcgacagtgcccccaagcctgaagaatatgcactcagtcaatggcatttgggggtagggag
ggggtgtagtgggcactgatatttttcagtctctgggtcaccacaagtttattaaaataaaagaaaatgtgggtaaatgctgccatctgtgctgt
ccctactcccaatacacacacacaggcacacacacacacaggcacacacacacacaggcacacatatacacacacacacacacacaca
cacacacacacacacacacacacacacgcataacaaatcaggtcccagactagatgcaggagttgaaactgctttaaaacactctaagaac
ttaactacaaacccacactccccatgcttctggggcttaagatctttgagctcattcttcagcatctctccacgagaaagtggggtgggctgctc
catgaggtggaggtgaagacccctgagtctgccccgtggagggggaggccccctaagcctagagtcccgctgcagggctctgtgccag
gagcccccgtgagccatggcctcgaaagggcagcggtgatttttttcacataaatatatcgcacttaaatgagtttagacagcatgacatcag
agagtaattaaattggtttgggttggaattccgtttccaattcctgagttcaggtttgtaaaagatttttctgagcacctgcaggcctgtgagtgtgt
gtgtgtgtgtgtgtgtgtgtgtgtgtgtgaagtattttcactggaaaggattcaaaactagggggaaaaaaaaactggagcacacaggcag
cattacgccattcttccttcttggaaaaatccctcagccttatacaagcctccttcaagccctcagtcagttgtgcaggagaaagggggcggtt
ggctttctcctttcaagaacgagttattttcagctgctgactggagacggtgcacgtctggatacgagagcatttccactatgggactggatac
aaacacacacccggcagacttcaagagtctcagactgaggagaaagcctttccttctgctgctactgctgctgccgctgcttttgaaagtcca
ctcctttcatggtttttcctgccaaaccagaggcacctttgctgctgccgctgttctctttggtgtcattcagcggctggccagaggatg
GDF5K→R mutant**
(wild type GDF5: Accession Number AAH32495)

(SEQ ID NO: 6)

MRLPKLLTFLLWYLAWLDLEFICTVLGAPDLGQRPQGTRPGLAKAEAKERPPLARNVFR
PGGHSYGGGATNANARAKGGTGQTGGLTQPKKDEPKKLPPRPGGPEPKPGHPPQTRQA
TARTVTPKGQLPGGKAPPKAGSVPSSFLLKKAREPGPPREPKEPFRPPPITPHEYMLSLYR
TLSDADRKGGNSSVKLEAGLANTITSFIDKGQDDRGPVVRKQRYVFDISALEKDGLLGA
ELRILRKKPSDTAKPAAPGGGRAAQLKLSSCPSGRQPASLLDVRSVPGLDGSGWEVFDI
WKLFRNFKNSAQLCLELEAWERGRAVDLRGLGFDRAARQVHEKALFLVFGRTKKRDL
FFNEIKARSGQDDKTVYEYLFSQRRRRRAPLATRQGKRPSKNLKARCSRKALHVNFKD
MGWDDWIIAPLEYEAFHCEGLCEFPLRSHLEPTNHAVIQTLMNSMDPESTPPTCCVPTRL
SPISILFIDSANNVVYKQYEDMVVESCGCR
**The K→R mutation is indicated in bold font above.
GDF5 preproprotein (K→R mutant)***
(wild type GDF5 preproprotein: Accession Number NP_000548)

(SEQ ID NO: 7)

MRLPKLLTFLLWYLAWLDLEFICTVLGAPDLGQRPQGTRPGLAKAEAKERPPLARNVFR
PGGHSYGGGATNANARAKGGTGQTGGLTQPKKDEPKKLPPRPGGPEPKPGHPPQTRQA
TARTVTPKGQLPGGKAPPKAGSVPSSFLLKKAREPGPPREPKEPFRPPPITPHEYMLSLYR
TLSDADRKGGNSSVKLEAGLANTITSFIDKGQDDRGPVVRKQRYVFDISALEKDGLLGA
ELRILRKKPSDTAKPAAPGGGRAAQLKLSSCPSGRQPASLLDVRSVPGLDGSGWEVFDI
WKLFRNFKNSAQLCLELEAWERGRAVDLRGLGFDRAARQVHEKALFLVFGRTKKRDL
FFNEIKARSGQDDKTVYEYLFSQRRRRRAPLATRQGKRPSKNLKARCSRKALHVNFKD
MGWDDWIIAPLEYEAFHCEGLCEFPLRSHLEPTNHAVIQTLMNSMDPESTPPTCCVPTRL
SPISILFIDSANNVVYKQYEDMVVESCGCR
***The K→R mutation is indicated in bold font above.
Furin
(Accession Number NP_002560)

(SEQ ID NO: 8)

MELRPWLLWVVAATGTLVLLAADAQGQKVFTNTWAVRIPGGPAVANSVARKHGFLNL
GQIFGDYYHFWHRGVTKRSLSPHRPRHSRLQREPQVQWLEQQVAKRRTKRDVYQEPTD
PKFPQQWYLSGVTQRDLNVKAAWAQGYTGHGIVVSILDDGIEKNHPDLAGNYDPGASF
DVNDQDPDPQPRYTQMNDNRHGTRCAGEVAAVANNGVCGVGVAYNARIGGVRMLD
GEVTDAVEARSLGLNPNHIHIYSASWGPEDDGKTVDGPARLAEEAFFRGVSQGRGGLGS
IFVWASGNGGREHDSCNCDGYTNSIYTLSISSATQFGNVPWYSEACSSTLATTYSSGNQN
EKQIVTTDLRQKCTESHTGTSASAPLAAGIIALTLEANKNLTWRDMQHLVVQTSKPAHL
NANDWATNGVGRKVSHSYGYGLLDAGAMVALAQNWTTVAPQRKCIIDILTEPKDIGK
RLEVRKTVTACLGEPNHITRLEHAQARLTLSYNRRGDLAIHLVSPMGTRSTLLAARPHD
YSADGFNDWAFMTTHSWDEDPSGEWVLEIENTSEANNYGTLTKFTLVLYGTAPEGLPV
PPESSGCKTLTSSQACVVCEEGFSLHQKSCVQHCPPGFAPQVLDTHYSTENDVETIRASV
CAPCHASCATCQGPALTDCLSCPSHASLDPVEQTCSRQSQSSRESPPQQQPPRLPPEVEA
GQRLRAGLLPSHLPEVVAGLSCAFIVLVFVTVFLVLQLRSGFSFRGVKVYTMDRGLISYK
GLPPEAWQEECPSDSEEDEGRGERTAFIKDQSAL
(Accession Number AAB28140)

(SEQ ID NO: 9)

KPAHLNANDWATNGVGRKVSHSYGYGLWTQAPWWPWPRIGPQWPPSGSASSTSSPSP
KTSGNGSRCGRP
(Accession Number AAH12181)

(SEQ ID NO: 10)

MELRPWLLWVVAATGTLVLLAADAQGQKVFTNTWAVRIPGGPAVANSVARKHGFLNL
GQIFGDYYHFWHRGVTKRSLSPHRPRHSRLQREPQVQWLEQQVAKRRTKRDVYQEPTD
PKFPQQWYLSGVTQRDLNVKAAWAQGYTGHGIVVSILDDGIEKNHPDLAGNYDPGASF
DVNDQDPDPQPRYTQMNDNRHGTRCAGEVAAVANNGVCGVGVAYNARIGGVRMLD
GEVTDAVEARSLGLNPNHIHIYSASWGPEDDGKTVDGPARLAEEAFFRGVSQGRGGLGS
IFVWASGNGGREHDSCNCDGYTNSIYTLSISSATQFGNVPWYSEACSSTLATTYSSGNQN
EKQIVTTDLRQKCTESHTGTSASAPLAAGIIALTLEANKNLTWRDMQHLVVQTSKPAHL
NANDWATNGVGRKVSHSYGYGLLDAGAMVALAQNWTTVAPQRKCIIDILTEPKDIGK
RLEVRKTVTACLGEPNHITRLEHAQARLTLSYNRRGDLAIHLVSPMGTRSTLLAARPHD
YSADGFNDWAFMTTHSWDEDPSGEWVLEIENTSEANNYGTLTKFTLVLYGTAPEGLPV
PPESSGCKTLTSSQACVVCEEGFSLHQKSCVQHCPPGFAPQVLDTHYSTENDVETIRASV
CAPCHASCATCQGPALTDCLSCPSHASLDPVEQTCSRQSQSSRESPPQQQPPRLPPEVEA
GQRLRAGLLPSHLPEVVAGLSCAFIVLVFVTVFLVLQLRSGFSFRGVKVYTMDRGLISYK
GLPPEAWQEECPSDSEEDEGRGERTAFIKDQSAL
SPC6
(Accession Number AAA91807)

(SEQ ID NO: 11)

LLCVLALLGGCLLPVCRTRVYTNHWAVKIAGGFPEANRIASKYGFINIGQIGALKDYYH
FYHSRTIKRSVISSRGTHSFISMEPKVEWIQQQVVKKRTKRDYDFSRAQSTYFNDPKWPS
MWYMHCSDNTHPCQSDMNIEGAWKRGYTGKNIVVTILDDGIERTHPDLMQNYDALAS
CDVNGNDLDPMPRYDASNENKHGTRCAGEVAAAANNSHCTVGIAFNAKIGGVRMLDG
DVTDMVEAKSVSFNPQHVHIYSASWGPDDDGKTVDGPAPLTRQAFENGVRMGRRGLG
SVFVWASGNGGRSKDHCSCDGYTNSIYTISISSTAESGKKPWYLEECSSTLATTYSSGES
YDKKIITTDLRQRCTDNHTGTSASAPMAAGIIALALEANPFLTWRDVQHVIVRTSRAGHL
NANDWKTNAAGFKVSHLYGFGLMDAEAMVMEAEKWTTVPRQHVCVESTDRQIKTIRP
NSAVRSIYKASGCSDNPNRHVNYLEHVVVAITITHPRRGDLAIYLTSPSGTRSQLLANRL
FDHSMEGFKNWEFMTIHCWGERAAGDWVLEVYDTPSQLRNFKTPGKLKEWSLVLYGT
SVQPYSPTNEFPKVERFRYSRVEDPTDDYGTEDYAGPCDPECSEVGCDGPGPDHCNDCL
HYYYKLKNNTRICVSSCPPGHYHADKKRCRKCAPNCESCFGSHGDQCMSCKYGYFLNE
ETNSCVTHCPDGSYQDTKKNLCRKCSENCKTCTEFHNCTECRDGLSLQGSRCSVSCEDG
RYFNGQDCQPCHRFCATCAGAGADGCINCTEGYFMEDGRCVQSCSISYYFDHSSENGY
KSCKKCDISCLTCNGPGFKNCTSCPSGYLLDLGMCQMGAICKDATEESWAEGGFCMLV
KKNNLCQRKVLQQLCCKTCTFQG
(Accession Number AAC50643)

(SEQ ID NO: 12)

MDWESRCCCPGRLDLLCVLALLGGCLLPVCRTRVYTNHWAVKIAGGFPEANRIASKYG
FINIGQIGALKDYYHFYHSRTIKRSVISSRGTHSFISMEPKVEWIQQQVVKKRTKRDYDSS
RVQSTYFNDPKWPSMWYMHCSDNTHPCQSDMNIEGAWKRGYTGKNIVVTILDDGIER
THPDLMQNYDALASCDVNGNDLDPMPRYDASNENKHGTRCAGEVAAAANNSHCTVGI
AFNAKIGGVRMLDGDVTDMVEAKSVSFNPQHVHIYSASWGPDDDGKTVDGPAPLTRQ
AFENGVRMGRRGLGSVFVWASGNGGRSKDHCSCDGYTNSIYTISISSTAESGKKPWYLE
ECSSTLATTYSSGESYDKKIITTDLRQRCTDNHTGTSASAPMAAGIIALALEANPFLTWR
DVQHVIVRTSRAGHLNANDWKTNAAGFKVSHLYGFGLMDAEAMVMEAEKWTTVPRQ
HVCVESTDRQIKTIRPNSAVRSIYKASGCSDNPNRHVNYLEHVVVRITITHPRRGDLAIYL
TSPSGTRSQLLANRLFDHSMEGFKNWEFMTIHCWGERAAGDWVLEVYDTPSQLRNFKT
PGKLKEWSLVLYGTSVRPYSPTNEFPKVERFRYSRVEDPTDDYGTEDYAGPCDPECSEV
GCDGPGPDHCNDCLHYYYKLKNNTRICVSSCPPGHYHADKKRCRKCAPNCESCFGSHG
DQCMSCKYGYFLNEETNSCVTHCPDGSYQDTKKNLCRKCSENCKTCTEFHNCTECRDG
LSLQGSRCSVSCEDGRYFNGQDCQPCHRFCATCAGAGADGCINCTEGYFMEDGRCVQS
CSISYYFDHSSENGYKSCKKCDISCLTCNGPGFKNCTSCPSGYLLDLGMCQMGAICKDA
TEESWAEGGFCMLVKKNNLCQRKVLQQLCCKTCTFQG
(Accession Number AAH12064)

(SEQ ID NO: 13)

MGWGSRCCCPGRLDLLCVLALLGGCLLPVCRTRVYTNHWAVKIAGGFPEANRIASKYG
FINIGQIGALKDYYHFYHSRTIKRSVISSRGTHSFISMEPKVEWIQQQVVKKRTKRDYDFS
RAQSTYFNDPKWPSMWYMHCSDNTHPCQSDMNIEGAWKRGYTGKNIVVTILDDGIER
THPDLMQNYDALASCDVNGNDLDPMPRYDASNENKHGTRCAGEVAAAANNSHCTVGI
AFNAKIGGVRMLDGDVTDMVEAKSVSFNPQHVHIYSASWGPDDDGKTVDGPAPLTRQ
AFENGVRMGRRGLGSVFVWASGNGGRSKDHCSCDGYTNSIYTISISSTAESGKKPWYLE
ECSSTLATTYSSGESYDKKIITTDLRQRCTDNHTGTSASAPMAAGIIALALEANPFLTWR
DVQHVIVRTSRAGHLNANDWKTNAAGFKVSHLYGFGLMDAEAMVMEAEKWTTVPRQ
HVCVESTDRQIKTIRPNSAVRSIYKASGCSDNPNRHVNYLEHVVVRITITHPRRGDLAIYL
TSPSGTRSQLLANRLFDHSMEGFKNWEFMTIHCWGERAAGDWVLEVYDTPSQLRNFKT
PGKLKEWSLVLYGTSVQPYSPTNEFPKVERFRYSRVEDPTDDYGTEDYAGPCDPECSEV
GCDGPGPDHCNDCLHYYYKLKNNTRICVSSCPPGHYHADKKRCRKCAPNCESCFGSHG
DQCMSCKYGYFLNEETNSCVTHCPDGSYQDTKKNLCRKCSENCKTCTEFHNCTECRDG
LSLQGSRCSVSCEDGRYFNGQDCQPCHRFCATCAGAGADGCINCTEGYFMEDGRCVQS
CSISYYFDHSSENGYKSCKKCDISCLTCNGPGFKNCTSCPSGYLLDLGMCQMGAICKDA
TEESWAEGGFCMLVKKNNLCQRKVLQQLCCKTCTFQG
(Accession Number NP_006191)

(SEQ ID NO: 14)

MGWGSRCCCPGRLDLLCVLALLGGCLLPVCRTRVYTNHWAVKIAGGFPEANRIASKYG
FINIGQIGALKDYYHFYHSRTIKRSVISSRGTHSFISMEPKVEWIQQQVVKKRTKRDYDFS
RAQSTYFNDPKWPSMWYMHCSDNTHPCQSDMNIEGAWKRGYTGKNIVVTILDDGIER
THPDLMQNYDALASCDVNGNDLDPMPRYDASNENKHGTRCAGEVAAAANNSHCTVGI
AFNAKIGGVRMLDGDVTDMVEAKSVSFNPQHVHIYSASWGPDDDGKTVDGPAPLTRQ
AFENGVRMGRRGLGSVFVWASGNGGRSKDHCSCDGYTNSIYTISISSTAESGKKPWYLE
ECSSTLATTYSSGESYDKKIITTDLRQRCTDNHTGTSASAPMAAGIIALALEANPFLTWR
DVQHVIVRTSRAGHLNANDWKTNAAGFKVSHLYGFGLMDAEAMVMEAEKWTTVPRQ
HVCVESTDRQIKTIRPNSAVRSIYKASGCSDNPNRHVNYLEHVVVRITITHPRRGDLAIYL
TSPSGTRSQLLANRLFDHSMEGFKNWEFMTIHCWGERAAGDWVLEVYDTPSQLRNFKT
PGKLKEWSLVLYGTSVQPYSPTNEFPKVERFRYSRVEDPTDDYGTEDYAGPCDPECSEV
GCDGPGPDHCNDCLHYYYKLKNNTRICVSSCPPGHYHADKKRCRKCAPNCESCFGSHG
DQCMSCKYGYFLNEETNSCVTHCPDGSYQDTKKNLCRKCSENCKTCTEFHNCTECRDG
LSLQGSRCSVSCEDGRYFNGQDCQPCHRFCATCAGAGADGCINCTEGYFMEDGRCVQS
CSISYYFDHSSENGYKSCKKCDISCLTCNGPGFKNCTSCPSGYLLDLGMCQMGAICKDA
TEESWAEGGFCMLVKKNNLCQRKVLQQLCCKTCTFQG

Each recited range includes all combinations and sub-combinations of ranges, as well as specific numerals contained therein.

All publications and patent applications cited in this specification are herein incorporated by reference in their entirety for all purposes as if each individual publication or patent application were specifically and individually indicated to be incorporated by reference for all purposes.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

1. A recombinant polynucleotide comprising a human Cartilage Derived Morphogenetic Protein-1 (hCDMP-1) variant or homolog thereof, wherein the recombinant polynucleotide is
(a) a polynucleotide that has the sequence of SEQ ID NO: 3;
(b) a polynucleotide that hybridizes under stringent hybridization conditions to (a) and encodes an amino acid sequence of SEQ ID NO: 4; or
(c) a polynucleotide that is a functional fragment of an amino acid sequence of SEQ ID NO: 4, or a conservatively modified variant of the functional fragment of the amino acid sequence of SEQ ID NO: 4;
wherein the polynucleotide encodes a polypeptide that directs the formation of normal joint structures.

2. The recombinant polynucleotide of claim 1 wherein the normal joint structures include cartilage, ligaments and tendons.

3. The recombinant polynucleotide of claim 1 encoding a polypeptide comprising the sequence of SEQ ID NO: 4.

4. The recombinant polynucleotide of claim 1 comprising SEQ ID NO: 3 or its complement.

5. The recombinant polynucleotide of claim 1 comprising SEQ ID NO: 4 or its complement.

6. A vector comprising the recombinant polynucleotide of claim 1.

7. An expression vector comprising the recombinant polynucleotide of claim 1 operatively linked to a regulatory sequence that controls expression of the polynucleotide in a host cell.

8. The expression vector of claim 7 wherein the recombinant polynucleotide is operatively linked to the regulatory sequence in an antisense orientation.

9. The expression vector of claim 7 wherein the recombinant polynucleotide is operatively linked to the regulatory sequence in a sense orientation.

10. A host cell comprising the recombinant polynucleotide of claim 1, or progeny of the cell.

11. The host cell of claim 10 that is a prokaryote.

12. The host cell of claim 10 that is a eukaryote.

13. A host cell comprising the recombinant polynucleotide of claim 1 operatively linked with a regulatory sequence that controls expression of the polynucleotide in a host cell.

14. The host cell of claim 13 wherein the nucleic acid is operatively linked to the regulatory sequence in an antisense orientation.

15. The expression vector of claim 13 wherein the nucleic acid is operatively linked to the regulatory sequence in a sense orientation.

16. An isolated DNA that encodes a hCDMP-1 protein variant as shown in SEQ ID NO: 4.

17. An antisense oligonucleotide complementary to a messenger RNA comprising SEQ ID NO: 3 and encoding a hCDMP-1 variant or homolog thereof, wherein the oligonucleotide inhibits the expression of hCDMP-1.

18. The recombinant polynucleotide of claim 1 that is RNA.

19. A method of producing a polypeptide comprising:
(i) culturing the host cell of claim 13 under conditions such that the polypeptide is expressed; and
(ii) recovering the polypeptide from the cultured host cell of its cultured medium.

20. A polypeptide encoded by a polynucleotide of claim 1 (a) or (b).

21. The polypeptide of claim 20 that has the amino acid sequence of SEQ ID NO: 4.

22. The polypeptide of claim 20 that is soluble.

23. The polypeptide of claim 20 that is fused with a heterologous peptide.

24. A pharmaceutical composition comprising a polynucleotide of claim 1, or a polypeptide of claim 20 and a pharmaceutically acceptable carrier.

25. A recombinant expression system comprising: the recombinant polynucleotide of claim 1.

26. A recombinant expression system for endoproteolytic processing of a hCDMP-1 protein variant comprising:
a) a first nucleotide sequence encoding a hCDMP-1 protein variant having the amino acid sequence as set forth in SEQ ID NO: 4 or conservative substitution thereof;
b) a second nucleotide sequence encoding SPC1; and
c) a third nucleotide sequence encoding SPC6;
wherein the first, second and third nucleotide sequences are independently operatively linked to transcription controlling nucleotide sequences in a host cell.

27. The recombinant expression system of claim 26, wherein the host cell is an autologous cell.

28. The recombinant expression system of claim 26, wherein the host cell is an allogeneic cell.

29. The recombinant expression system of claim 26, wherein the host cell is a functional progenitor cell capable of differentiating into skeletal tissue.

30. The recombinant expression system of claim 29, wherein the host cell is a chondrocyte progenitor cell.

31. The recombinant expression system of claim 29, wherein the skeletal tissue is cartilage, bone, ligament, or tendon.

32. The recombinant expression system of claim 26, wherein the host cell is isolated from the synovium, periosteum, perichondrium, or other source of cells capable of differentiating into skeletal tissue.

33. A method of modulating musculoskeletal disorders in a subject, the method comprising the step of administering to the subject a therapeutically effective amount of a nucleic acid encoding the recombinant polynucleotide of claim 1.

34. The method of claim 33, comprising the step of administering to the subject a therapeutically effective amount of a second nucleic acid encoding SPC1 and a third nucleic acid encoding SPC6.

35. A method of modulating musculoskeletal disorders in a subject, the method comprising the step of administering to the subject a therapeutically effective amount of a nucleic acid encoding a hCDMP-1 polypeptide variant, wherein the nucleic acid hybridizes under stringent conditions to a nucleic acid encoding a polypeptide having an amino acid sequence of SEQ ID NO: 4.

36. A method for modulating musculoskeletal disorders in a subject comprising the steps of: (a) isolating cells to be implanted into said subject (b) introducing into the cells the recombinant expression system of claim 26; and (c) implanting the cells containing the recombinant expression system into said subject.

37. The method of claim 36, wherein the cells express wildtype hCDMP-1.

38. The method of claim 36, wherein the cells do not express wildtype hCDMP-1.

39. The method of claim 36, wherein the cells are functional progenitor cells.

40. The method of claim 39, wherein the functional progenitor cells are chondrocyte progenitor cells.

41. The method of claim 40, wherein the cells are isolated from the synovium, periosteum, perichondrium, or other source of cells capable of differentiating into skeletal tissue.

42. A method for modulating musculoskeletal disorders in a subject in need thereof, comprising:
(a) selecting the patient in need thereof;
(b) isolating cells from the patient, wherein the cells express CDMP-1 and introducing into the cells a first nucleotide sequence encoding SPC1 and a second nucleotide sequence encoding SPC6, wherein the first and second nucleotide sequences are independently operatively linked to transcription controlling nucleotide sequences in the isolated cells; and
(c) readministering the cells to the patient.

43. A method for modulating musculoskeletal disorders in a subject in need thereof, comprising:
(a) selecting the patient in need thereof;
(b) isolating cells from the patient, wherein the cells do not express CDMP-1; and
introducing into the cells the recombinant expression system of claim 26; and
(c) readministering the cells to the patient.

44. The method of claim 43, wherein the cells are functional progenitor cells.

45. The method of claim 44, wherein the functional progenitor cells are chondrocyte progenitor cells.

46. The method of claim 40, wherein the cells are isolated from the synovium, periosteum, perichondrium, or other source of cells capable of differentiating into skeletal tissue.
